(19) United States
(12) Patent Application Publication
(10) Pub. No.: US 2025/0322956 A1
(43) Pub. Date: Oct. 16, 2025

(54) MACHINE LEARNING TO PREDICT PATIENT OUTCOMES BASED ON POSITIONING

(71) Applicant: MatrixCare, Inc., Bloomington, MN (US)

(72) Inventors: Elizabeth Boyd SILVA, Burlington, MA (US); James Francis RANOUS, Apex, NC (US); Darlene Michele ALEKSZA, Apex, NC (US)

(21) Appl. No.: 19/169,272

(22) Filed: Apr. 3, 2025

Related U.S. Application Data
(60) Provisional application No. 63/634,659, filed on Apr. 16, 2024.

(51) Int. Cl. G16H 50/20; G16H 40/67; G16H 50/30

(52) U.S. Cl. CPC G16H 50/20; G16H 40/67; G16H 50/30

(57) ABSTRACT

Techniques for improved machine learning are provided. Sensor data collected by a set of sensors is accessed, the sensor data indicating positioning of a patient in a physical environment. A set of patient characteristics for the patient is determined. An outcome score for the positioning of the patient is generated, using a trained machine learning model, based on the sensor data and the set of patient characteristics. In response to determining that the outcome score does not satisfy one or more criteria, one or more interventions are initiated for the patient.

### CROSS-REFERENCE TO RELATED APPLICATIONS

This application claims priority to U.S. Provisional Patent Application No. 63/634,659, filed Apr. 16, 2024, the entire content of which is incorporated herein by reference in its entirety.

### INTRODUCTION

Embodiments of the present disclosure relate to machine learning. More specifically, embodiments of the present disclosure relate to using machine learning to predict action performance quality.

A wide variety of healthcare providers (e.g., caregivers, nurses, doctors, and the like) currently provide a similarly wide variety of care for patients to assist with a vast array of disorders, diseases, disabilities, or other medical concerns. In many cases, home healthcare has become increasingly common (and preferred by patients), such as when a caregiver assists the patient in their own home. Similar assistance is often provided in healthcare facilities, including hospitals, in-patient clinics, residential care facilities, and the like. Generally, healthcare providers learn how to perform various assistance actions in a variety of ways, such as through lessons or instruction, by observing more experienced providers perform the actions, and the like. In most cases, once a provider learns how to perform some action, they do not receive further instruction or oversight when performing the action subsequently.

Improved systems and techniques to predict action quality are needed.

### SUMMARY

According to some embodiments presented in this disclosure, a method is provided. The method includes: accessing first sensor data collected by a set of sensors, the first sensor data indicating movement of a first user in a physical environment; determining an action that the first user was performing when the first sensor data was collected, wherein the first user was performing the action to assist a patient; generating a quality score for performance of the action, by the first user, based on processing the first sensor data using a trained machine learning model; and in response to determining that the quality score does not satisfy one or more criteria, initiating one or more interventions for the first user.

According to some embodiments presented in this disclosure, a method is provided. The method includes: accessing first sensor data collected by a set of sensors, the first sensor data indicating movement of a first user in a physical environment; determining an action that the first user was performing when the first sensor data was collected, wherein the first user was performing the action to assist a patient; training a machine learning model to generate quality scores for performance of the action based on the first sensor data; and deploying the machine learning model to generate quality scores.

According to some embodiments presented in this disclosure, a method is provided. The method includes: accessing first sensor data collected by a set of sensors, the first sensor data indicating positioning of a patient in a physical environment; determining a set of patient characteristics for the patient; generating an outcome score for the positioning of the patient, using a first trained machine learning model, based on the first sensor data and the set of patient characteristics; and in response to determining that the outcome score does not satisfy one or more criteria, initiating one or more interventions for the patient.

According to some embodiments presented in this disclosure, a method is provided. The method includes: accessing first sensor data collected by a set of sensors, the first sensor data indicating positioning of a patient in a physical environment; determining a set of patient characteristics for the patient; training a machine learning model to generate outcome scores for patient positioning based on the first sensor data and the set of patient characteristics; and deploying the machine learning model to generate outcome scores.

According to some embodiments presented in this disclosure, a method is provided. The method includes: accessing first sensor data collected by a set of sensors, the first sensor data indicating movement of a first user in a physical environment; determining an action that the first user was performing when the first sensor data was collected, wherein the first user was performing the action to assist a patient; generating an injury risk score for performance of the action, by the first user, based on processing the first sensor data using a trained machine learning model, wherein the injury risk score indicates a risk of injury to the first user; and in response to determining that the injury risk score satisfies one or more criteria, initiating one or more interventions for the first user.

According to some embodiments presented in this disclosure, a method is provided. The method includes: accessing first sensor data collected by a set of sensors, the first sensor data indicating movement of a first user in a physical environment; determining an action that the first user was performing when the first sensor data was collected, wherein the first user was performing the action to assist a patient; training a machine learning model to generate injury risk scores for performance of the action based on the first sensor data; and deploying the machine learning model to generate quality scores.

Other embodiments provide processing systems configured to perform the aforementioned methods as well as those described herein; non-transitory, computer-readable media comprising instructions that, when executed by one or more processors of a processing system, cause the processing system to perform the aforementioned methods as well as those described herein; and a computer program product embodied on a computer-readable storage medium comprising code for performing the aforementioned methods as well as those further described herein.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example environment for collecting and evaluating sensor data, according to some embodiments of the present disclosure.

FIG. 2 depicts an example workflow for training machine learning models to predict action performance quality, according to some embodiments of the present disclosure.

FIG. 3 depicts an example workflow for refining machine learning models to predict action performance quality for specific users, according to some embodiments of the present disclosure.

FIG. 4 depicts an example workflow for using machine learning models to predict action performance quality, according to some embodiments of the present disclosure.

FIG. 5 depicts an example workflow for training machine learning models to predict patient outcomes based on sensor data, according to some embodiments of the present disclosure.

FIG. 6 depicts an example workflow for training a set of machine learning models to predict patient position and patient outcomes, according to some embodiments of the present disclosure.

FIG. 7 depicts an example workflow for refining machine learning models to predict patient outcomes for specific patients, according to some embodiments of the present disclosure.

FIG. 8 depicts an example workflow for using machine learning models to predict patient outcomes, according to some embodiments of the present disclosure.

FIG. 9 depicts an example workflow for training machine learning models to predict action injury risk, according to some embodiments of the present disclosure.

FIG. 10 depicts an example workflow for refining machine learning models to predict action injury risk for specific users, according to some embodiments of the present disclosure.

FIG. 11 depicts an example workflow for using machine learning models to predict action injury risk, according to some embodiments of the present disclosure.

FIG. 12 is a flow diagram depicting an example method for training machine learning models to predict action performance quality, according to some embodiments of the present disclosure.

FIG. 13 is a flow diagram depicting an example method for generating training data for machine learning models to predict action performance quality, according to some embodiments of the present disclosure.

FIG. 14 is a flow diagram depicting an example method for refining machine learning models to predict action performance quality for specific users, according to some embodiments of the present disclosure.

FIG. 15 is a flow diagram depicting an example method for using machine learning models to predict action performance quality, according to some embodiments of the present disclosure.

FIG. 16 is a flow diagram depicting an example method for generating interventions based on machine learning evaluations of action performance, according to some embodiments of the present disclosure.

FIG. 17 is a flow diagram depicting an example method for training machine learning models to predict patient outcomes based on sensor data, according to some embodiments of the present disclosure.

FIG. 18 is a flow diagram depicting an example method for generating training data for machine learning models to predict patient outcomes based on sensor data, according to some embodiments of the present disclosure.

FIG. 19 is a flow diagram depicting an example method for training a set of machine learning models to predict patient position and patient outcomes, according to some embodiments of the present disclosure.

FIG. 20 is a flow diagram depicting an example method for training a set of machine learning models to predict relevant patient outcomes, according to some embodiments of the present disclosure.

FIG. 21 is a flow diagram depicting an example method for refining machine learning models to predict patient outcomes for specific patients, according to some embodiments of the present disclosure.

FIG. 22 is a flow diagram depicting an example method for using machine learning models to predict patient outcomes based on sensor data, according to some embodiments of the present disclosure.

FIG. 23 is a flow diagram depicting an example method for using a set of machine learning models to predict patient position and patient outcomes, according to some embodiments of the present disclosure.

FIG. 24 is a flow diagram depicting an example method for using a set of machine learning models to predict relevant patient outcomes, according to some embodiments of the present disclosure.

FIG. 25 is a flow diagram depicting an example method for generating interventions based on machine learning evaluations of patient outcomes, according to some embodiments of the present disclosure.

FIG. 26 is a flow diagram depicting an example method for training machine learning models to predict action injury risk, according to some embodiments of the present disclosure.

FIG. 27 is a flow diagram depicting an example method for training a set of machine learning models to predict relevant injury risks, according to some embodiments of the present disclosure.

FIG. 28 is a flow diagram depicting an example method for generating training data for machine learning models to predict action injury risk, according to some embodiments of the present disclosure.

FIG. 29 is a flow diagram depicting an example method for refining machine learning models to predict action injury risk for specific users, according to some embodiments of the present disclosure.

FIG. 30 is a flow diagram depicting an example method for using machine learning models to predict action injury risk, according to some embodiments of the present disclosure.

FIG. 31 is a flow diagram depicting an example method for using a set of machine learning models to predict relevant user injury risks, according to some embodiments of the present disclosure.

FIG. 32 is a flow diagram depicting an example method for generating interventions based on machine learning evaluations of action injury risk, according to some embodiments of the present disclosure.

FIG. 33 is a flow diagram depicting an example method for training action-specific machine learning models, according to some embodiments of the present disclosure.

FIG. 34 is a flow diagram depicting an example method for using action-specific machine learning models, according to some embodiments of the present disclosure.

FIG. 35 is a flow diagram depicting an example method for accessing sensor data for machine learning, according to some embodiments of the present disclosure.

FIG. 36 is a flow diagram depicting an example method for learning to predict action performance quality, according to some embodiments of the present disclosure.

FIG. 37 is a flow diagram depicting an example method for predicting action performance quality, according to some embodiments of the present disclosure.

FIG. 38 is a flow diagram depicting an example method for learning to predict patient outcomes based on sensor data, according to some embodiments of the present disclosure.

FIG. 39 is a flow diagram depicting an example method for predicting patient outcomes based on sensor data, according to some embodiments of the present disclosure.

FIG. 40 is a flow diagram depicting an example method for learning to predict action injury risk, according to some embodiments of the present disclosure.

FIG. 41 is a flow diagram depicting an example method for predicting action injury risk, according to some embodiments of the present disclosure.

FIG. 42 depicts an example computing device configured to predict and/or learn to predict action performance quality according to various aspects of the present disclosure.

FIG. 43 depicts an example computing device configured to predict and/or learn to predict patient outcomes various aspects of the present disclosure.

FIG. 44 depicts an example computing device configured to predict and/or learn to predict action injury risk various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved machine learning to evaluate user movements and positioning.

In some embodiments of the present disclosure, sensor information from a variety of sensors can be collected and evaluated, using one or more machine learning models, to evaluate the performance of the actions. For example, a machine learning model may be trained to predict or quantify the quality of the action performance. For example, the model(s) may be used to predict or determine whether the user is performing the action correctly, whether the user is likely to sustain injury (or re-injury) based on the action performance, whether the patient being assisted is likely to sustain any complications or concerns based on the action performance, and the like. As used herein, “user” is generally inclusive of any individual providing care, such as a caregiver, nurse, family member of the patient, doctor, and the like. Similarly, “patient” is generally inclusive of any individual that receives care, such as a patient in a hospital or clinic, an individual living in a residential care facility, an elderly or disabled recipient of home healthcare, and the like.

Generally, the particular information collected and analyzed to evaluate action performance may vary depending on the particular implementation. In some embodiments, the data includes sensor data collected via one or more sensors in the physical space (e.g., room) where the action is being performed. For example, the sensor data may include data collected by one or more wearable devices on the patient and/or the user (e.g., smart watches, cameras, microphones, and the like) and/or one or more sensors installed in the room where the action(s) are performed (e.g., cameras, microphones, pressure sensors, and the like). In some embodiments, for example, the sensor data may include accelerometer data captured using one or more accelerometer sensors (e.g., indicating acceleration of the user's hands, arms, or other body parts as they perform the action, and/or indicating the acceleration of one or more parts of the patient as the action is performed). As another example, the sensor data may include orientation data collected using one or more orientation sensors (e.g., indicating the orientation of the user's hands, arms, or other body parts as they perform the action, and/or indicating the orientation of one or more parts of the patient as the action is performed and/or after the action is completed).

As another example, the sensor data may include pressure data collected using one or more pressure sensors (e.g., indicating the amount of pressure that the caregiver is exerting on the patient and/or objects or surfaces while performing the action, and/or indicating the amount of pressure exerted on the patient (e.g., by the caregiver or by other objects such as their bed) as the action is performed and/or after the action is completed). As another example, the sensor data may include video data and/or image data collected using one or more imaging sensors (e.g., cameras observing the caregiver and/or patient as the action is performed and/or after the action is completed). As another example, the sensor data may include audio data collected using one or more audio sensors (e.g., microphones observing the caregiver and/or patient as the action is performed and/or after the action is completed).

In some embodiments, the sensor data is accessed and evaluated in response to determining that the caregiver is performing an action (which may include determining that the user is about to perform the action) that can (or should) be evaluated using the machine learning models. Generally, determining that the user is performing the action may include a wide variety of operations, such as receiving an indication of the action (e.g., where the user presses a button, verbally indicates the action, or otherwise indicates that they are about to perform the action). For example, the user may use their smartphone or wearable device to indicate that they are beginning an action. In some embodiments, the system may infer or determine action performance indirectly. For example, the system may evaluate video or image data (e.g., using convolutional neural networks) to detect particular patterns or movements indicative of performing given actions.

In some embodiments, some (or all) of the sensors may be in a deactivated state prior to determining that the action is being performed. The sensor(s) may be turned off or otherwise not collecting or generating any data, or may be configured to automatically delete any collected data after a relatively short period of time (e.g., every second, every five seconds, and the like) without storing it (e.g., without storing it locally, or without transmitting it to a central server). In some aspects, this latter embodiment may allow the system to use the sensors themselves to detect initiation of the action (e.g., using machine learning to evaluate audio in order to detect defined words or phrases, such as “Starting patient transfer” or “beginning monitored action”). If such triggers are not identified, the sensor data can be deleted. If the trigger(s) are identified, the data (and additional data) may be collected, stored, or otherwise provided for further analysis.

In some embodiments, after determining that the action is being performed, the system may activate the sensor(s) in the space to observe the action performance. As used herein, “activating” sensors generally corresponds to placing them in an active state, such as by turning them on, instructing them to begin storing and/or transmitting sensor data, accessing sensor data they generated, and the like. In some embodiments, when the system determines that the user has completed performance of the action(s), the system may automatically return the sensor(s) to the deactivated state. As above, determining that the user has finished performing the action may include a wide variety of operations, such as receiving an indication of the completion (e.g., where the user presses a button, verbally states that the action is complete, or otherwise indicates that they have finished performing the action). For example, the user may use their smartphone or wearable device to indicate that they have finished an action. In some embodiments, the system may infer or determine action completion indirectly. For example, the system may evaluate video or image data (e.g., using convolutional neural networks) to detect particular patterns or movements indicative of completion of given actions.

This dynamic sensor activation and deactivation can substantially improve user and patient privacy, as sensor data may only be collected and/or evaluated during relatively brief windows. Further, the dynamic sensor activation and deactivation can substantially reduce compute requirements and storage footprint of the system. For example, by only selectively generating and/or accessing the sensor data when actions are being performed, the amount of data that needs to be stored and/or processed can be substantially reduced. This enables the system to operate with reduced memory and storage requirements, as well as reduced computational expense (e.g., reduced processor time).

As discussed above, the various sensor data may be processed by a wide variety of machine learning models in order to generate a wide variety of predictions. For example, in some aspects, the data may be analyzed to predict the quality of the action performance (e.g., to generate a quality score for the performance). As used herein, performance quality generally corresponds to whether the user performed the action in accordance with a preferred or designated manner (e.g., whether the user acted in accordance with their training, medical guidelines, patient preferences and/or a defined “correct” way to perform the action). For example, if the user performed the action differently than instructed (or contrary to medical guidance), the quality score may indicate poor performance. Notably, in some embodiments, the quality score may be distinct from action completion. That is, the score may be low even if the action was otherwise completed successfully. In some embodiments, failure to successfully complete the action may similarly result in a low action quality score.

As another example, in some aspects, the data may be analyzed to predict patient outcomes based on the action performance (e.g., to generate an outcome score for the performance). As used herein, the outcome score generally corresponds to what outcome(s) the patient is predicted to experience based on performance of the action (e.g., whether the patient is expected to improve, worsen, or remain the same). For example, based on how the user positioned, lifted, carried, or otherwise interacted with the patient, the outcome score may indicate whether the patient may suffer injuries such as pressure sores, bruising, fractured bones, and the like. In some embodiments, the outcome scores may additionally or alternatively indicate whether the patient will be comfortable in the positioning.

As yet another example, in some aspects, the data may be analyzed to predict user injury risks based on the action performance (e.g., to generate an injury score for the performance). As used herein, the injury score generally corresponds to what outcome(s) the user is predicted to experience based on performance of the action (e.g., whether the user may injure their back, aggravate an existing injury, and the like). For example, based on how the user positioned, lifted, carried, or otherwise interacted with the patient, the injury score may indicate whether the user may suffer injuries such as back injury, knee injury, wrist strain, and the like. In some embodiments, the sensor data is evaluated to predict injury risks based on return-to-work conditions (e.g., in response to determining that the user recently returned to work from a prior injury), in order to predict the risk of additional injury (e.g., to determine whether the user is ready for returning to work, to predict whether certain actions should be performed by other users, and the like).

As used herein, “actions” can generally include any actions performed by a user to assist, treat, diagnose, or otherwise interact with a patient. For example, the actions may include helping the patient stand, sit, lay down, walk, get into or out of locations or objects, and the like. Similarly, the actions may include helping the patient bathe, cat, and the like. Further, the actions may include medical actions such as removing old bandages, cleaning and bandaging a wound, inserting a catheter, and the like.

In some embodiments, based on the various machine learning-based predictions, the system may take a variety of actions and/or initiate a variety of interventions. As one example, the system may transmit a notification or alert to the user performing the action, to a supervisor of the user, and the like. For example, if the system predicts that the action quality is low, a notification to the user (indicating the action) may prompt them to reconsider how they performed the action to improve future performance. Similarly, a notification to the user's supervisor may prompt the supervisor to check in with the user to improve training. In some embodiments, the system may transmit or provide tutorial material (e.g., written material, audio and/or video instructions, and the like) to the user. This can allow the user to immediately review the proper technique(s) to perform the action(s), ensuring improvement.

As another example, if the system predicts that the patient outcome will be poor, a notification to the user (indicating the action) may prompt them to perform the action again (e.g., to reposition the patient) to improve their outcomes, and/or to improve future performance of the action (e.g., to perform differently in the future to reduce the chance of harm). Similarly, a notification to the user's supervisor may prompt the supervisor to check in with the user to improve future performance. In some embodiments, the system may transmit or provide tutorial material (e.g., written material, audio and/or video instructions, and the like) to the user in response to the outcome score. This can allow the user to review the proper technique(s) to perform the action(s), reducing potential harm.

As yet another example, if the system predicts that the injury risk to the user is sufficiently high, a notification to the user (indicating the action) may prompt them to refrain from performing the action again (e.g., to prevent re-injury), and/or to take additional care when performing the action (e.g., to perform differently in the future to reduce the chance of injury). Similarly, a notification to the user's supervisor may prompt the supervisor to check in with the user to determine whether the user is ready to return to work (e.g., after injury) or if they need more time to heal.

In some aspects, the system can take a variety of actions or interventions to improve user and/or patient outcomes or treatments. For example, based on predicting that action performance quality is low, the system may infer that the user is at risk or that something else should be investigated (e.g., perhaps the user performed the action poorly not because they need training, but because they are distracted or tired). In response, the system may prompt or schedule a break for the user, or perform other actions to investigate and/or assist. For example, the user may have been distracted due to the specific environment or tasks (e.g., the particular patient may require substantial help, and would be better served by having multiple caregivers attend). This can substantially improve user morale as well as patient and user outcomes.

As another example, based on predicting the patient outcomes are low, the system may improve future patient outcomes by prompting the user to perform the action again and/or differently. In some embodiments, the system may additionally or alternatively improve patient outcomes, such as by suggesting particular treatment options to ameliorate the concerns, ordering particular treatment materials (e.g., shipping bandages or other material to the patient's residence in response to predicting a risk of pressure sores), and the like.

As yet another example, based on predicting that the user is at risk of injury (or re-injury), the system can improve outcomes by preventing such injury (e.g., instructing the user to perform the action differently or not at all, assigning another user to perform the action in the future, and the like). In these and other ways, aspects of the present disclosure can substantially improve treatments and outcomes.

### Example Environment for Collecting and Evaluating Sensor Data

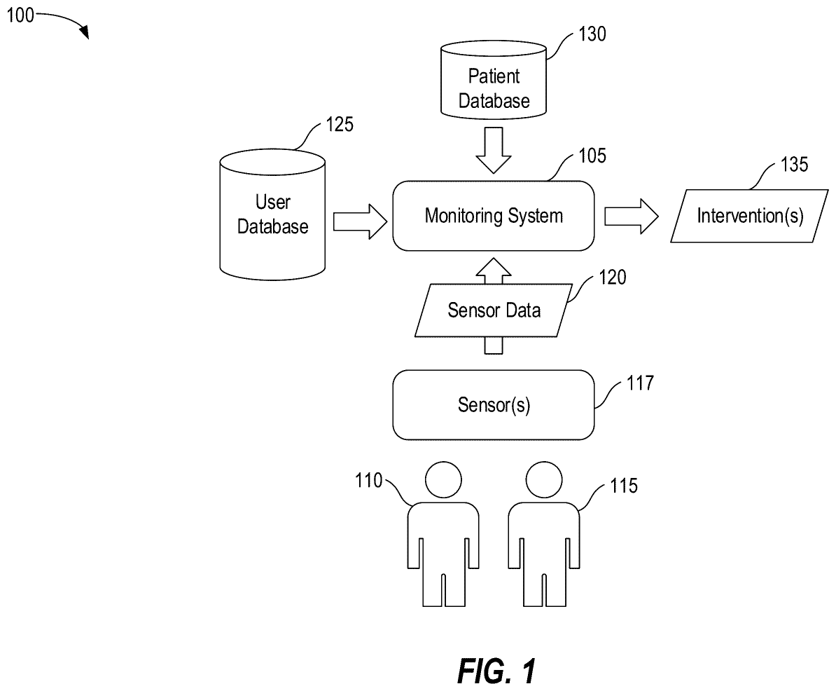

FIG. 1 depicts an example environment 100 for collecting and evaluating sensor data, according to some embodiments of the present disclosure.

In the illustrated example, a monitoring system 105 accesses a variety of data, including information from a user database, information from a patient database, and sensor data 120 in order to dynamically generate interventions 135 using machine learning model(s). As used herein, “accessing” data may generally include receiving, requesting, retrieving, generating, collecting, obtaining, or otherwise gaining access to the data. Though the monitoring system 105 is depicted as a discrete system, in some embodiments some or all of the functionality of the monitoring system 105 may be implemented across any number of systems. Generally, the monitoring system 105 represents a computing system (which may be implemented using hardware, software, or a combination of hardware and software) configured to use machine learning to evaluate sensor data, as discussed in more detail below.

In the illustrated example, a set of sensors 117 are used to generate or collect sensor data 120 associated with one or more individuals (such as a user 110 and a patient 115). As discussed above, the user 110 is generally representative of a healthcare provider (e.g., a nurse, a caregiver, a doctor, and the like). In some embodiments, the user 110 provides care, but is not strictly a healthcare provider. For example, the user 110 may be a family member or friend of the patient 115. The patient 115 is generally representative of any individual that the user 110 assists (e.g., a resident in a residential care facility, a patient in a hospital, and the like). Although a single user 110 and patient 115 are depicted for conceptual clarity, in embodiments, there may be any number of users and patients associated with the sensors 117.

In the illustrated example, the sensors 117 are generally representative of a wide variety of sensor devices or components, where the sensors 117 are configured to capture or collect data in a physical environment occupied by the user 110 and/or patient 115. In embodiments, the particular architecture of the sensors 117 may vary depending on the particular implementation. For example, in some embodiments, the sensors 117 include one or more wearable devices worn by the user 110 and/or the patient 115 (e.g., smart watches). In some embodiments, the sensors 117 include one or more pressure sensors (e.g., on or in the floor and/or furniture in a room). In some embodiments, the sensors 117 include one or more audio sensors (e.g., microphones), imaging sensors (e.g., video cameras), and the like. Generally, any sensor device capable of collecting useful data in the environment (e.g., data that can be evaluated to predict action quality, patient outcomes and/or positioning, and/or injury risk) may be used.

In some embodiments, some or all of the sensors 117 are provided and/or installed by one or more healthcare entities (e.g., the employer of the user 110). For example, a healthcare facility may install sensors 117 in hospital room(s) where patients reside, may provide wearable sensors 117 to employees, and the like.

In the illustrated example, the sensors 117 provide sensor data 120 to the monitoring system 105. In some embodiments, some or all of the sensors 117 provide the sensor data 120 in a “push” operation. That is, as the sensors 117 collected data, they may transmit the sensor data 120 to the monitoring system 105 without waiting for a request. In some embodiments, some or all of the sensors 117 provide the sensor data 120 in a “pull” operation. That is, the sensors 117 may generate and store the sensor data locally and transmit it to the monitoring system 105 in response to receiving a request from the monitoring system 105.

In some aspects, as discussed above, some or all of the sensors 117 may be in a deactivated state until they are triggered to begin data collection. As one example, some or all of the sensors 117 may generate or collet sensor data (e.g., audio), and this sensor data may be evaluated (e.g., by the sensors 117, by the monitoring system 105, or by another component) to determine whether to trigger or transition the sensor(s) 117 to the active state. For example, in response to determining (e.g., based on the sensor data) that the user 110 is beginning performance of an action (e.g., because the user verbally announces the action), the monitoring system 105 may cause the sensors 117 to enter an active state, where the sensor data is collected and provided to the monitoring system 105 (e.g., saved and/or used for processing). If the sensor(s) 117 are in the inactive state, the data may be discarded after a relatively brief period of time (e.g., refraining from storing the data).

In some embodiments, some or all of the sensors 117 may not collect any data while in the deactivated state. For example, a camera sensor may be turned off (e.g., power not provided to the imaging sensor), a physical shutter or other blocking component may be used to block the sensor (e.g., a shutter in front of the camera), and the like.

Generally, the sensors 117 may provide the sensor data 120 to the monitoring system 105 using any suitable communications medium. For example, the sensors 117 and the monitoring system 105 may be communicatively coupled using one or more links (which may include one or more hardware links and/or one or more wireless links). In some embodiments, some or all of the sensors 117 provide the sensor data 120 via one or more networks (e.g., a wireless local area network (WLAN), the Internet, and the like).

As discussed above, the sensor data 120 may generally include any information useable to predict action performance quality, patient positioning and/or outcomes, and/or injury (or re-injury) risk. For example, in some embodiments, the sensor data 120 may include accelerometer data (e.g., from one or more wearable sensors on the hand and/or wrist of the user 110), orientation data (e.g., indicating the orientation of the user's hands and/or arms, the orientation of the patient 115, and the like), pressure data (e.g., indicating where the user 110 and/or patient 115 are in the room, indicating the amount of pressure being exerted on the patient 115 and/or user 110, and the like), video data and/or image data (e.g., depicting the user 110 performing the action), audio data (e.g., recording audio of the user 110 and/or patient 115), and the like.

In the illustrated example, the monitoring system 105 also accesses data from a user database 125 and a patient database 130. Although depicted as two discrete repositories for conceptual clarity, in some aspects, the information in the user database 125 and patient database 130 may be combined or distributed across any number of repositories. Further, although depicted as independent from the monitoring system 105, in some aspects, some or all of the data may be stored locally by the monitoring system 105.

In some embodiments, the user database 125 generally includes information about user(s) 110. For example, the user database 125 may store information such as what training materials each user 110 has received, what action(s) they are able to perform, whether they have suffered any injuries (and characteristics of such injuries, such as when they occurred, the nature of the injury, and the like). In some embodiments, the user database 125 includes information about prior predictions for the user 110. For example, each time the monitoring system 105 generates an action quality score for an action performed by the user 110, the monitoring system 105 may update the user database 125 to reflect the score, to generate an average quality score for the user over time, and the like. This may allow the monitoring system 105 (or other systems) to determine the average quality of a user with respect to any given action, to identify any trends in the performance quality of time and/or at different times of day, and the like. In some embodiments, the user database 125 may similarly store predictions related to injury risk for the user 110.

In some embodiments, the patient database 130 generally includes information about patient(s) 115. For example, the patient database 130 may store information such as what diseases or disorders each patient 115 has, what action(s) they receive assistance with, whether they have suffered any injuries, their medications, comorbidities (e.g., medical conditions), demographics, and the like. In some embodiments, the patient database 130 includes information about prior predictions for the patient 115. For example, each time the monitoring system 105 generates an outcome score for the patient 115, the monitoring system 105 may update the patient database 130 to reflect the score, to generate an average outcome score for the patient over time, and the like. This may allow the monitoring system 105 (or other systems) to determine whether the predictions are accurate, to refine the machine learning models, and the like.

In the illustrated example, the monitoring system 105 evaluates some or all of the sensor data 120, data from the user database 125, and/or data from the patient database 130 using one or more machine learning models in order to generate interventions 135. In some embodiments, as discussed above, three models (or three sets of models) may be used to generate three predictions for each action performed to assist the patient 115: a first prediction relating to the quality of the action performance (e.g., how well the user 110 performed the action), a second prediction relating to the predicted outcome(s) for the patient 115 based on the performance (e.g., based on the positioning in which the user 110 placed the patient 115), and/or a third prediction relating to the potential risk of injury (or re-injury) by the user 110 in performing the action.

In some embodiments, the monitoring system 105 may perform a variety of preprocessing operations on the sensor data 120 and/or user data and patient data prior to processing it using the machine learning model(s). For example, in some embodiments, the monitoring system 105 may delineate the sensor data 120 into a sequence of windows (e.g., into non-overlapping five second windows) and process each window separately. In some embodiments, the monitoring system 105 may perform various feature extractions on the data, such as to determine the mean, maximum values, minimum values, derivative(s), and the like. In some embodiments, the machine learning model(s) receive and evaluate a time series of data (e.g., corresponding to acceleration and orientation over time for each hand). Generally, the particular architecture of the model(s) may differ depending on the particular implementation. For example, for image data, the monitoring system 105 may use one or more convolutional neural networks (CNNs) to identify objects (e.g., the user's hands), track movement through physical space (e.g., to monitor how the user's hands move in three-dimensional space), and the like.

In some embodiments, the monitoring system 105 initiates the interventions 135 based on the generated predictions. For example, the monitoring system 105 may determine whether each prediction satisfies one or more criteria (e.g., meets a threshold value). As one example, if the predicted action quality is below a threshold, the monitoring system 105 may initiate interventions 135 such as transmitting one or more instructions or tutorials to the user 110. As another example, if the predicted outcome score for the patient 115 is below a threshold (e.g., indicating a poor outcome) and/or above a threshold (e.g., indicating a high probability of a negative outcome), the monitoring system 105 may notify the user 110 to act differently next time and/or adjust the patient 115 now (e.g., to adjust how they are positioned). As another example, if the predicted injury risk score exceeds a threshold (e.g., indicating a high probability of injury), the monitoring system 105 may notify the user 110 to refrain from performing that action again.

In some embodiments, multiple thresholds or criteria may be used to evaluate an action. For example, if the quality is below a first threshold, the monitoring system 105 may notify the user 110. If the quality is below a second (lower) threshold, the monitoring system 105 may provide an instruction or tutorial. If the quality is below a third (even lower) threshold, the monitoring system 105 may notify the user's supervisor, assign additional training for the user 110, and the like.

As discussed above, the interventions 135 may generally include a wide variety of actions, including providing notifications or alerts to one or more users 110 or patients 115, providing instructional material, suggesting alternative treatments, and the like. By dynamically generating such interventions 135, the monitoring system 105 is able to substantially improve the results and outcomes for the patients 115 and users 110.

### Example Workflow to Train Machine Learning Models to Predict Action Performance Quality

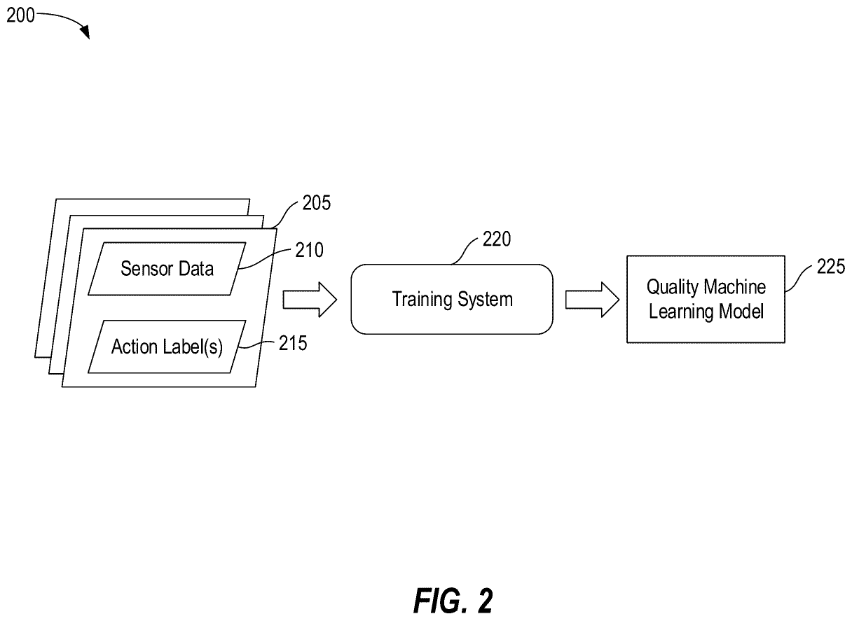

FIG. 2 depicts an example workflow 200 for training machine learning models to predict action performance quality, according to some embodiments of the present disclosure.

In the illustrated example, a training system 220 accesses training data 205 to generate (e.g., train) one or more quality machine learning models 225. Though the training system 220 is depicted as a discrete system, in some embodiments some or all of the functionality of the training system 220 may be implemented across any number of systems. Generally, the training system 220 represents a computing system (which may be implemented using hardware, software, or a combination of hardware and software) configured to train and/or update machine learning models to evaluate sensor data, as discussed in more detail below. In some embodiments, the training system 220 is implemented on the same system as the monitoring system 105 discussed above with reference to FIG. 1.

In the illustrated example, the training data 205 includes a set of training records (also referred to in some aspects as exemplars, samples, or action exemplars), where each record comprises sensor data 210 and a corresponding set of one or more action labels 215. The sensor data 210 (which may correspond to the sensor data 120 of FIG. 1) generally corresponds to data collected or measured using one or more sensors as a user (e.g., the user 110 of FIG. 1) performs an action (e.g., to assist a patient such as the patient 115 of FIG. 1). For example, as discussed above, the sensor data 210 may include acceleration and/or orientation data from one or more wearable sensors worn by the user, image data from one or more imaging sensors observing the user performing the action, and the like.

In the illustrated example, the action label(s) 215 generally indicate a ground-truth quality of the performed action. For example, the action label 215 may indicate whether the action (which corresponds to the sensor data 210) was performed correctly (e.g., using a binary label). In some aspects, the action label 215 is a continuous value (e.g., a score between zero and one) indicating the quality of the performance,

As discussed above, in some embodiments, the quality of the performance (e.g., the “correctness” of the action) is determined based on a defined or preferred technique (e.g., in accordance with medical guidance). For example, the training data 205 may include exemplars created by collecting sensor data as an “expert” or experienced user performs the action according to protocol (where such samples are associated with a label indicating high quality) and/or exemplars created by collecting sensor data while the experienced user intentionally performs the action incorrectly (e.g., performing it poorly, inaccurately, or in ways that inexperienced users often act), where such samples may be associated with a label indicating poor performance quality.

In some embodiments, the action quality is defined based at least in part on the accuracy or precision of movement of the user (e.g., the acceleration, orientation, and/or position of the user's hands (e.g., relative to the patient) at one or more points in time during the performance). That is, action performance which is more similar to the “ideal” performance may be labeled with high scores, while data that is dissimilar from the target are given low scores. In some embodiments, the action quality is defined at least in part on the speed of the movement (e.g., such that performance of the action should take a defined period of time, and actions completed substantially faster or substantially slower may be scored poorly). In some embodiments, the action labels 215 are generated based at least in part on subjective feedback (e.g., from the patient for whom the action was performed) about the quality of the performance.

For example, to generate the training data 205, the training system 220 (or another system) may collect the sensor data 210 while a user performs an action, and then generate an action label 215 to score the action performance (e.g., by asking an expert and/or the patient to score the performance). Generally, the action labels 215 (and the particular modalities reflected in the sensor data 210) may vary depending on the particular implementation and action type. The sensor data 210 generally includes any data relevant to determining the quality of the particular action, and the action labels 215 may be defined using any criteria relevant to the particular action. For example, the acceleration of the user's hands may relevant for one action (e.g., to determine how well the action was performed) and irrelevant for another. Similarly, the physical pressure exerted by the user may be relevant for some actions and irrelevant for others.

In the illustrated example, the training system 220 generates a quality machine learning model 225 using the training data 205. Generally, the particular operations and techniques used to train the quality machine learning model 225 may vary depending on the particular model architecture and implementation. For example, in the case of neural networks, the training system 220 may process the sensor data 210 of a given sample of training data 205 as input to the quality machine learning model 225 in order to generate an output prediction of performance quality. This prediction can then be compared against the corresponding action label 215 of the sample in order to generate a loss (e.g., using cross-entropy loss), and the loss can be used to update one or more model parameters (e.g., using backpropagation). This process may be performed iteratively for each training exemplar independently (e.g., using stochastic gradient descent) and/or in batches (e.g., using batch gradient descent).

Although a single quality machine learning model 225 is depicted for conceptual clarity, in some embodiments, the training system 220 may train an ensemble of models. For example, in some embodiments, a different quality machine learning model 225 may be trained to process each modality of sensor data 210 (e.g., a first model to generate a prediction based on the accelerometer data, a second model to generate a prediction based on the image data, and so on). These modality-specific predictions may then be aggregated (e.g., summed, averaged, or used as input to a final scoring machine learning model to predict the performance quality).

As another example, in some embodiments, the training system 220 may train a different quality machine learning model 225 for each action type. For example, a first model may be trained to predict the quality of a first action performance (e.g., bandaging a wound) while a second model may be trained to predict the quality of a different action (e.g., placing a catheter) and a third model is trained to predict the quality of a third action (e.g., drawing blood), and so on.

In some aspects, once the quality machine learning model(s) 225 are trained, the training system 220 may deploy them for inferencing. As used herein, deploying the model may generally include any operations used to prepare or provide the model to process input data (e.g., new sensor data) during runtime. For example, the training system 220 may instantiate the model locally, or may transmit the model details (e.g., the values of the learned parameters) to one or more other systems for inferencing.

### Example Workflow to Refine Machine Learning Models to Predict Action Performance Quality for Specific Users

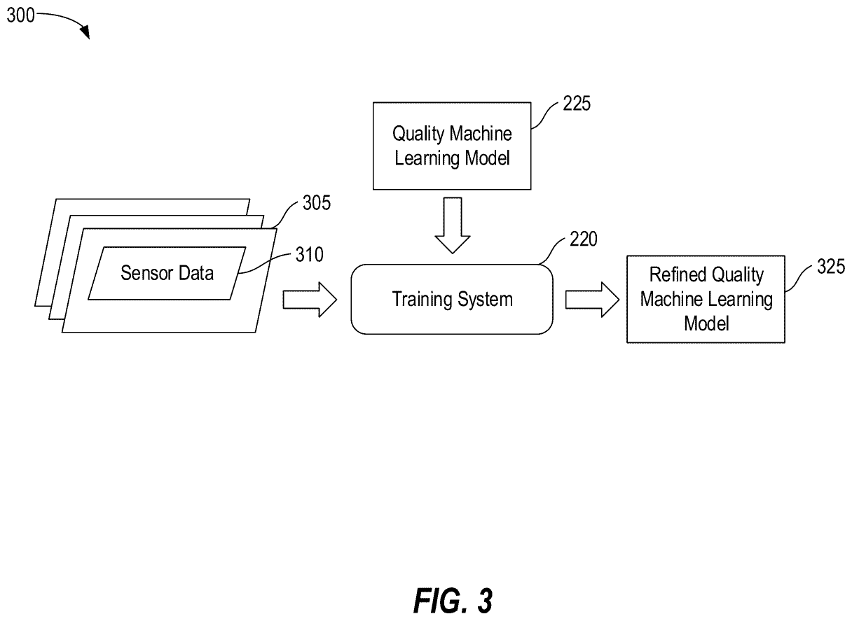

FIG. 3 depicts an example workflow 300 for refining machine learning models to predict action performance quality for specific users, according to some embodiments of the present disclosure.

In the illustrated example, a training system 220 (which may be the same as the training system 220 of FIG. 2, or may be a different system) accesses personalization data 305 to generate (e.g., train or refine) one or more refined quality machine learning models 325. As discussed above, though the training system 220 is depicted as a discrete system, some or all of the functionality of the training system 220 may be implemented across any number of systems, and the training system 220 may be implemented using hardware, software, or a combination of hardware and software. In the illustrated example, the training system 220 is configured to update or personalize machine learning models to evaluate sensor data for individual users, as discussed in more detail below. In some embodiments, the training system 220 is implemented on the same system as the monitoring system 105 discussed above with reference to FIG. 1.

In the illustrated example, the personalization data 305 includes a set of training records (also referred to in some aspects as exemplars, samples, or personalization exemplars), where each record comprises sensor data 310. The sensor data 310 (which may correspond to the sensor data 120 of FIG. 1 and/or the sensor data 210 of FIG. 2) generally corresponds to data collected or measured using one or more sensors as a user (e.g., the user 110 of FIG. 1) performs an action (e.g., to assist a patient such as the patient 115 of FIG. 1). For example, as discussed above, the sensor data 310 may include acceleration and/or orientation data from one or more wearable sensors worn by the user, image data from one or more imaging sensors observing the user performing the action, and the like.

In the illustrated example, the sensor data 310 corresponds to a single user. That is, while the sensor data 210 of FIG. 2 may depict action performance by any number of users (e.g., using a large number of samples from a large number of users to improve training and generalization of the models), the personalization data 305 may include exemplars of a single specific user performing action(s).

For example, to generate the personalization data 305, the training system 220 (or another system) may collect the sensor data 310 while a given user performs actions. In some embodiments, as discussed above, the sensor data 310 generally includes any data relevant to determining the quality of the particular action. For example, the acceleration of the user's hands may relevant for one action (e.g., to determine how well the action was performed) and irrelevant for another. Similarly, the physical pressure exerted by the user may be relevant for some actions and irrelevant for others.

In some embodiments, the personalization data 305 is collected while the quality machine learning model 225 is being used to evaluate performance quality for the user. For example, after being trained (e.g., using the workflow 200 of FIG. 2), the quality machine learning model 225 may be used to evaluate performance quality while the user performs actions to assist patients. In some aspects, in addition to evaluating the sensor data 310, the training system 220 (or another system) may store the sensor data 310 for future training or refinement of the quality machine learning model 225. In some aspects, as the workflow 300 seeks to personalize the model for the specific user, the personalization data 305 may lack labels. In some aspects, the personalization data 305 may be labeled with the label that was generated for the sensor data 310 (by the quality machine learning model 225) during runtime. In some aspects, the personalization data 305 may be labeled in a similar manner to the training data 205 of FIG. 2. For example, during runtime, the training system 220 may collect feedback from the user, other experienced users observing the actions, the patient, and the like. This feedback may then be used to form labels for the sensor data 310.

In the illustrated example, the training system 220 generates a refined quality machine learning model 325 by updating the quality machine learning model 225 using the personalization data 305. Generally, the particular operations and techniques used to update or refine the quality machine learning model 225 may vary depending on the particular model architecture and implementation, as discussed above. For example, in the case of neural networks, the training system 220 may process the sensor data 310 of a given sample of personalization data 305 as input to the quality machine learning model 225 in order to generate an output prediction of performance quality. This prediction can then be compared against a corresponding label (e.g., against the label generated based on patient feedback, as discussed above) of the sample in order to generate a loss (e.g., using cross-entropy loss), and the loss can be used to update one or more model parameters (e.g., using backpropagation). This process may be performed iteratively for each training exemplar independently (e.g., using stochastic gradient descent) and/or in batches (e.g., using batch gradient descent).

In some embodiments, rather than training the refined quality machine learning model 325 based on external considerations (e.g., patient feedback, scoring by an experienced user, and the like), the training system 220 may label the sensor data 310 as high quality (e.g., in binary or continuous values) for the specific user. For example, after performing an action, the user may indicate that the action was performed well, or otherwise in accordance with how they prefer to perform. In response, the training system 220 may store the collected sensor data 310 for future refinement of the quality machine learning model 225 for the specific user.

Although a single refined quality machine learning model 325 is depicted for conceptual clarity, as discussed above, the training system 220 may generate an ensemble of personalized models in some embodiments. For example, in some embodiments, a different refined quality machine learning model 325 may be refined to process each modality of sensor data 310, a different refined quality machine learning model 325 may be trained for each action type, and the like.

In some aspects, once the refined quality machine learning model(s) 325 are trained, the training system 220 may deploy them for inferencing, as discussed above. For example, when processing future sensor data for the specific user, the training system 220 may cease use of the non-personalized quality machine learning model 225, and may instead process the new data using a refined quality machine learning model 325 that was updated specifically for the specific user based on sensor data 310 from the specific user.

### Example Workflow to Use Machine Learning Models to Predict Action Performance Quality

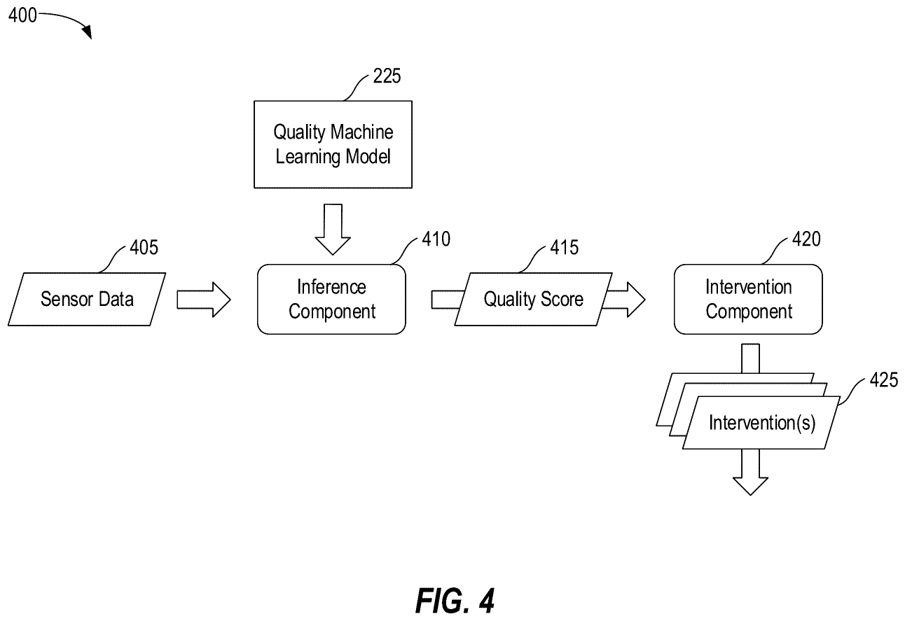

FIG. 4 depicts an example workflow 400 for using machine learning models to predict action performance quality, according to some embodiments of the present disclosure.

In the illustrated example, an inference component 410 and an intervention component 420 accesses sensor data 405 and one or more trained quality machine learning model(s) 225 to generate quality score(s) 415 and intervention(s) 425. Though the inference component 410 and the intervention component 420 are depicted as a discrete components for conceptual clarity, in some embodiments some or all of the functionality of the depicted components may be implemented across any number of systems (e.g., a single system). Generally, the inference component 410 and intervention component 420 represent one or more computing systems (which may be implemented using hardware, software, or a combination of hardware and software) configured to use quality machine learning models to evaluate sensor data and generate interventions, as discussed in more detail below. In some embodiments, the inference component 410 and the intervention component 420 are components of a monitoring system, such as the monitoring system 105 of FIG. 1, and/or of a training system, such as the training system 220 of FIG. 2.

In the illustrated example, the sensor data 405 (which may correspond to the sensor data 120 of FIG. 1, the sensor data 210 of FIG. 2, and/or the sensor data 310 of FIG. 3) generally corresponds to data collected or measured using one or more sensors as a user (e.g., the user 110 of FIG. 1) performs an action (e.g., to assist a patient such as the patient 115 of FIG. 1). For example, as discussed above, the sensor data 405 may include acceleration and/or orientation data from one or more wearable sensors worn by the user, image data from one or more imaging sensors observing the user performing the action, and the like.

In the illustrated example, the inference component 410 accesses the sensor data 405 and processes it using a quality machine learning model 225 to generate a quality score 415. Although a single quality machine learning model 225 is depicted for conceptual clarity, in some embodiments, the inference component 410 may use an ensemble of models (e.g., processing each modality of sensor data 405 using a different model, processing the sensor data 405 using a different model (or set of models) based on the action being performed, and the like). Further, although not included in the illustrated example, in some embodiments the inference component 410 may process the sensor data 405 using a personalized or customized model (e.g., the refined quality machine learning model 325 of FIG. 3) that has been personalized for the specific user depicted in the sensor data 405.

In some aspects, as discussed above, the inference component 410 accesses the sensor data 405 in response to one or more triggers, such as determining that the user is about to perform (or is performing) the action. For example, as discussed above, the user may press a button on their smartphone or a wearable device, verbally announce the action, and the like. In response, the inference component 410 may begin accessing the sensor data 405 (e.g., by activating one or more sensors in the room). In some embodiments, once the inference component 410 determines that the action is complete (e.g., when the user presses a button again, verbally announces completion, steps away from the patient, and the like), the inference component 410 can similarly deactivate the sensor(s).

Although not depicted for conceptual clarity, in some aspects, the inference component 410 may perform one or more preprocessing steps on the sensor data 405 to prepare the data to be used as input to the quality machine learning model 225, as discussed above.

The quality score 415 is generally representative of a prediction, generated by the quality machine learning model 225, indicating the quality of the performance of the action. In some embodiments, the quality score 415 is a categorical score (e.g., a binary value indicating whether the performance was good or poor). In some embodiments, the quality score 415 is (or includes) a continuous value or measure (e.g., scoring the performance between zero and one, where one represents perfect execution of the action).

In some embodiments, as discussed above, the quality score 415 may be stored in a repository (such as the user database 125 of FIG. 1), or may otherwise be used to update stored data for the user (e.g., to indicate the average performance quality of one or more actions by the user).

In the illustrated workflow 400, the quality score 415 is accessed by the intervention component 420. The intervention component 420 generally evaluates the quality score 415 using one or more criteria (e.g., rules and/or thresholds) to determine what intervention(s) 425, if any, to initiate. For example, in some embodiments, the intervention component 420 may determine whether the quality score 415 is above or below a defined threshold. If the quality score 415 is below a threshold, the intervention component 420 may determine to initiate one or more interventions 425. As discussed above, in some embodiments, the intervention component 420 may use multiple thresholds or rules to evaluate the quality score 415 and determine which intervention(s) 425 should be used.

For example, in some embodiments, the intervention component 420 may generate and transmit a notification to the user depicted by the sensor data 405, may transmit instructional or tutorial information to the user to assist them in improving, may generate and transmit a notification to the supervisor or manager of the user, and the like.

In some embodiments, in addition to or instead of generating interventions 425 based on a single quality score 415, the intervention component 420 may evaluate multiple such scores for the user. For example, the intervention component 420 may determine whether the last N quality scores 415 for the user fail to meet the criteria, or whether at least M % of the last K quality scores 415 for the user failed to meet the criteria. If not, the intervention component 420 may refrain from generating an intervention 425 (e.g., if the user's recent quality scores or average quality scores meet the criteria). If the recent trend indicates potential concerns, however, the intervention component 420 may initiate one or more interventions 425 (or may generate more involved interventions, such as notifying a superior or providing instructions, as compared to simply notifying the user).

In some embodiments, the interventions 425 may generally indicate relevant information such as the user that was performing the action, the action that was being performed, the date and/or time when the action was performed, the patient that was being assisted, and the like. In some embodiments, the notification may indicate the quality score 415 itself.

In some embodiments, in addition to determining which (if any) interventions 425 to initiate, the intervention component 420 may also determine which samples of sensor data 405 (if any) to save for future refinement of the quality machine learning model 225 (e.g., to generate a personalized model using the workflow 300 of FIG. 3). For example, the intervention component 420 may determine whether the quality score 415 meets or exceeds a threshold. If so (e.g., if the action was performed very well), the intervention component 420 may save the sensor data 405 to personalize or refine the model specifically for the given user.

Generally, the workflow 400 may be used whenever sensor data 405 is available and/or whenever the user performs an action, as discussed above. This can ensure reliable monitoring of the user's performance and improve the quality of assistance offered to patients.

### Example Workflow to Train Machine Learning Models to Predict Patient Outcomes Based on Sensor Data

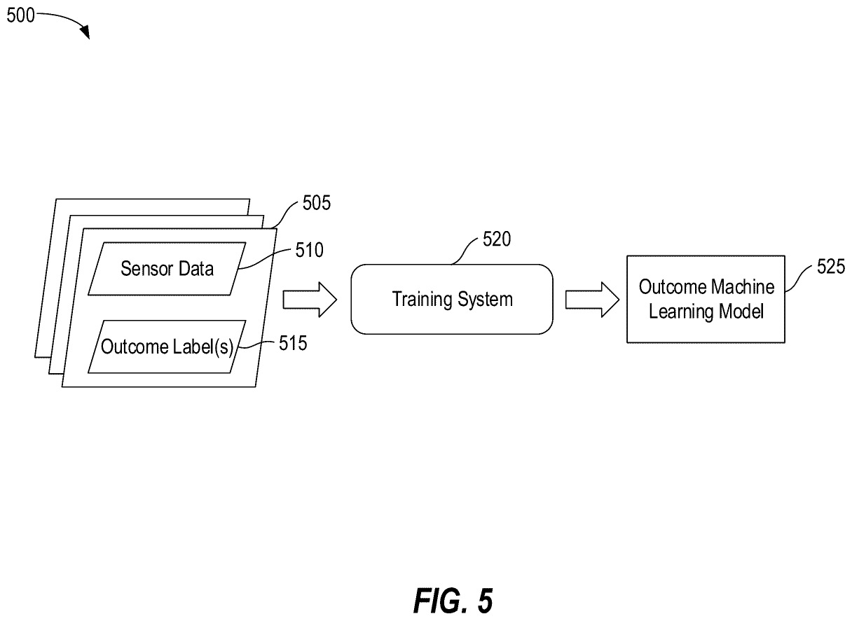

FIG. 5 depicts an example workflow 500 for training machine learning models to predict patient outcomes based on sensor data, according to some embodiments of the present disclosure.

In the illustrated example, a training system 520 accesses training data 505 to generate (e.g., train) one or more outcome machine learning models 525. Though the training system 520 is depicted as a discrete system, in some embodiments some or all of the functionality of the training system 520 may be implemented across any number of systems. Generally, the training system 520 represents a computing system (which may be implemented using hardware, software, or a combination of hardware and software) configured to train and/or update machine learning models to evaluate sensor data, as discussed in more detail below. In some embodiments, the training system 520 is implemented on the same system as the monitoring system 105 discussed above with reference to FIG. 1 and/or the training system 220 discussed above with reference to FIGS. 2-3.

In the illustrated example, the training data 505 includes a set of training records (also referred to in some aspects as exemplars, samples, or outcome exemplars), where each record comprises sensor data 510 and a corresponding set of one or more outcome labels 515. The sensor data 510 (which may correspond to the sensor data 120 of FIG. 1 and/or the sensor data 210 of FIG. 2) generally corresponds to data collected or measured using one or more sensors as a user (e.g., the user 110 of FIG. 1) performs an action (e.g., to assist a patient such as the patient 115 of FIG. 1). For example, as discussed above, the sensor data 510 may include acceleration and/or orientation data from one or more wearable sensors worn by the user, image data from one or more imaging sensors observing the user performing the action, and the like.

In the illustrated example, the outcome label(s) 515 generally indicate the outcome(s) experienced by the patient after the user performed the action. For example, the outcome label 515 may indicate whether the patient condition improved, worsened, or remained the same. As another example, the outcome label 515 may indicate specific medical conditions, such as whether the patient contracted pressure sores, recovered, or suffered any other condition or disorder. In some embodiments, the outcome labels 515 may indicate whether the positioning of the patient, as a result of the action, was comfortable for the patient (e.g., based on their conditions, demographics, and the like). For example, some positions may be comfortable for some patients but uncomfortable for others that have a different set of medical conditions. In some aspects, the outcome labels 515 only indicate outcomes that were caused by (or are believed to have been caused by) the action performance itself. For example, pressure sores may be caused by placing the patient in the same position for a relatively long period of time. Accordingly, if the action involved helping the patient move or transfer between chairs and/or beds, and the user left the patient in the same body position, it may be inferred that the way the action was performed (moving the patient but allowing them to remain in the same orientation or seated position) caused the condition (pressure sores). As another example of more direct causation, suppose the action involved lifting the patient to move them from the bed to a wheelchair, and the patient suffered bruising on their arms where the user lifted them. It may be concluded that the bruising (the outcome) was caused by the way the action (lifting the patient) was performed. Accordingly, an outcome label 515 may be generated indicating that the action performance (as reflected in the sensor data 510) caused the indicated negative outcome(s) (e.g., bruising).

In some aspects, the outcome labels 515 comprise one or more continuous values (e.g., a score between zero and one) indicating the severity of the outcomes (e.g., where large values indicate substantially negative outcomes, such as higher values for worse conditions and/or higher values for worse states of the same condition, such as worse bruising). In some embodiments, the outcome labels 515 comprise one or more binary values (e.g., indicating whether each outcome occurred).

As discussed above, in some embodiments, the outcome labels are determined based on defined mappings. For example, some outcomes (e.g., pressure sores, bruising, broken bones, and the like) may be defined as only occurring when one or more particular actions (e.g., transferring the patient) are performed incorrectly. In some embodiments, therefore, when such outcomes are noted, the training system 520 (or another system) may automatically evaluate patient data (e.g., from the patient database 130 of FIG. 1) to identify whether one or more of the corresponding action(s) were performed for the patient. In some aspects, this may include determining whether the action was performed within a timeline specified in the mapping (e.g., indicating the predicted or typical time that elapses between the action and the resulting outcome).

If such action(s) are found, the training system 520 (or another system) may automatically generate a training exemplar that labels the corresponding sensor data 510 (collected when the action or actions were performed) with the noted outcome(s). In this way, the system can automatically build a set of training data 505 based on defined action-outcome mappings, and this training data 505 can be used to train outcome machine learning models 525 to predict whether particular outcomes will occur based on how future action(s) are performed (e.g., based on sensor data collected while users perform the actions).

In some embodiments, the outcome labels 515 are additionally or alternatively defined based at least in part on manual review by experts or experienced users. For example, an experienced user (e.g., a supervisor) may observe another user performing an action (either in real-time or subsequently), such as by reviewing video or imagery of the action, reviewing sensor data such as pressure information, and the like. The experienced user may then indicate what negative outcome(s), if any, they predict will occur (e.g., while correcting the user if the action is being performed in real-time). In some embodiments, some or all of the training data 505 may be generated based on such manual evaluation and correction.

As another example, in some embodiments, the outcome labels 515 are additionally or alternatively generated based at least in part on subjective feedback (e.g., from the patient for whom the action was performed) about the quality of the performance. For example, to generate the training data 505, the training system 520 (or another system) may collect the sensor data 510 while a user performs an action, and then generate an outcome label 515 to score the action performance (e.g., by surveying the patient about any concerns, such as soreness or bruising). Generally, the outcome labels 515 (and the particular modalities reflected in the sensor data 510) may vary depending on the particular implementation and action type. The sensor data 510 generally includes any data relevant to determining the quality of the particular action and/or predicting future outcomes, and the outcome labels 515 may be defined using any criteria relevant to the particular outcomes and actions. For example, the physical pressure exerted by the user's hands may relevant for one action (e.g., to determine whether the pressure will cause bruising or other injury) and irrelevant for another. Similarly, the orientation in which the user leaves the patient may be relevant for some actions and outcomes, and irrelevant for others.

Although not depicted in the illustrated example, in some aspects, one or more other data sources may also be used as input to the model during training. For example, in some embodiments, data relating to the patient(s) being assisted (e.g., from the patient database 130) may be used as input to the model. For example, information related to the patient's height, weight, age, and the like may be useful inputs to predict outcome risks.

In the illustrated example, the training system 520 generates an outcome machine learning model 525 using the training data 505. Generally, the particular operations and techniques used to train the outcome machine learning model 525 may vary depending on the particular model architecture and implementation. For example, in the case of neural networks, the training system 520 may process the sensor data 510 of a given sample of training data 505 as input to the outcome machine learning model 525 in order to generate an output prediction of outcome(s) that may result from the performance. This prediction can then be compared against the corresponding outcome label 515 of the sample in order to generate a loss (e.g., using cross-entropy loss), and the loss can be used to update one or more model parameters (e.g., using backpropagation). This process may be performed iteratively for each training exemplar independently (e.g., using stochastic gradient descent) and/or in batches (e.g., using batch gradient descent).

Although a single outcome machine learning model 525 is depicted for conceptual clarity, in some embodiments, the training system 520 may train an ensemble of models. For example, in some embodiments, a different outcome machine learning model 525 may be trained to process each modality of sensor data 510 (e.g., a first model to generate a prediction based on the accelerometer data, a second model to generate a prediction based on the image data, and so on). These modality-specific predictions may then be aggregated (e.g., summed, averaged, or used as input to a final scoring machine learning model to predict the action outcomes).

As another example, in some embodiments, the training system 520 may train a different outcome machine learning model 525 for each outcome type (e.g., a first model trained to predict whether pressure sores will occur, a second model trained to predict whether bruising will occur, and the like). As yet another example, in some embodiments, the training system 520 may train a different outcome machine learning model 525 for each action type. For example, a first model may be trained to predict outcomes that may occur as a result of performance of a first action (e.g., bandaging a wound) while a second model may be trained to predict the potential outcomes of a different action (e.g., transferring the patient), a third model may be trained to predict the outcomes resulting from a third action (e.g., drawing blood), and so on.

In some aspects, once the outcome machine learning model(s) 525 are trained, the training system 520 may deploy them for inferencing. As discussed above, deploying the model may generally include any operations used to prepare or provide the model to process input data (e.g., new sensor data) during runtime. For example, the training system 520 may instantiate the model locally, or may transmit the model details (e.g., the values of the learned parameters) to one or more other systems for inferencing.

### Example Workflow to Train a Set of Machine Learning Models to Predict Patient Position and Patient Outcomes

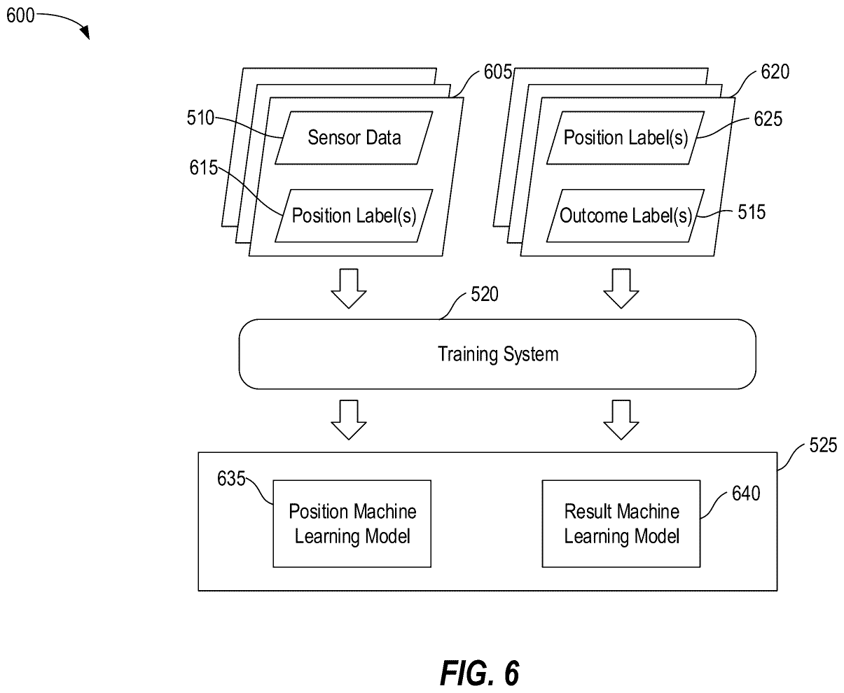

FIG. 6 depicts an example workflow 600 for training a set of machine learning models to predict patient position and patient outcomes, according to some embodiments of the present disclosure.

In the illustrated example, a training system 520 (which may be the same as the training system 520 of FIG. 5, or may be a different system) accesses training data 605 and/or 620 to generate (e.g., train) one or more position machine learning models and one or more result machine learning models 640. In the illustrated example, the position machine learning model 635 and the result machine learning model 640 form an ensemble model. That is, the position machine learning model 635 and the result machine learning model 640 may collectively form an outcome prediction model, such as the outcome machine learning model 525. Though the training system 520 is depicted as a discrete system, in some embodiments some or all of the functionality of the training system 520 may be implemented across any number of systems. Generally, the training system 520 represents a computing system (which may be implemented using hardware, software, or a combination of hardware and software) configured to train and/or update machine learning models to evaluate sensor data, as discussed in more detail below. In some embodiments, the training system 520 is implemented on the same system as the monitoring system 105 discussed above with reference to FIG. 1 and/or the training system 220 discussed above with reference to FIGS. 2-3.

In the illustrated example, the training data 605 includes a set of training records (also referred to in some aspects as exemplars, samples, or position exemplars), where each record comprises sensor data 510 and a corresponding set of one or more position labels 615. As discussed above, the sensor data 510 (which may correspond to the sensor data 120 of FIG. 1, the sensor data 210 of FIG. 2, and/or the sensor data 510 of FIG. 5) generally corresponds to data collected or measured using one or more sensors as a user (e.g., the user 110 of FIG. 1) performs an action (e.g., to assist a patient such as the patient 115 of FIG. 1). For example, as discussed above, the sensor data 510 may include acceleration and/or orientation data from one or more wearable sensors worn by the user, image data from one or more imaging sensors observing the user performing the action, and the like. In some embodiments, the sensor data 510 may indicate the positioning of the patient. For example, the sensor data 510 may be used to predict the position of the patient, or may directly indicate some or all aspects of the patient positioning (e.g., using pressure sensors in the bed of the patient).

In the illustrated example, the position label(s) 615 generally indicate the positioning of the patient (e.g., the patient 115 of FIG. 1) as a result of the user actions. That is, the position labels 615 may indicate what position the patient was in when the user completed the actions (as indicated in the sensor data 510). For example, the position labels 615 may indicate whether the patient was left standing, sitting, laying down, reclined, and the like. Further, in some aspects, the position labels 615 may indicate the orientation of the patient, such as whether they are on their back, stomach, left side, right side, and the like. In some aspects, the position labels 615 may indicate information about where the patient is positioned, such as seated in a chair with no back, seated in a chair with a back, seated in a hard chair or a soft chair, the height of the chair or bed, and the like.

In some aspects, the position labels 615 are generated manually by the user or patient. That is, the user and/or patient may indicate (e.g., via a personal device) what position the patient is in. In some aspects, some or all of the position labels 615 may be generated automatically, such as based on pressure sensors, cameras, or other sensors in the space. For example, the training system 520 (or another system) may detect which chair the patient is seated in using pressure sensors, or may detect which side the patient is laying on using image analysis.

In the illustrated example, the training system 520 generates a position machine learning model 635 using the training data 605. Although not depicted in the illustrated example, in some aspects, one or more other data sources may also be used as input to the model during training. For example, in some embodiments, data relating to the patient(s) being assisted (e.g., from the patient database 130) may be used as input to the model. For example, information related to the patient's existing conditions, comorbidities, capacity to move by themselves, and the like may be useful inputs to predict positioning.

Generally, the particular operations and techniques used to train the position machine learning model 635 may vary depending on the particular model architecture and implementation. For example, in the case of neural networks, the training system 520 may process the sensor data 510 of a given sample of training data 605 as input to the position machine learning model 635 in order to generate an output prediction of what position(s) the patient is likely to be left in, based on the performance. This prediction can then be compared against the corresponding position label 615 of the sample in order to generate a loss (e.g., using cross-entropy loss), and the loss can be used to update one or more model parameters (e.g., using backpropagation). This process may be performed iteratively for each training exemplar independently (e.g., using stochastic gradient descent) and/or in batches (e.g., using batch gradient descent).

Although a single position machine learning model 635 is depicted for conceptual clarity, in some embodiments, the training system 520 may train an ensemble of models. For example, in some embodiments, a different position machine learning model 635 may be trained to process each modality of sensor data 510 (e.g., a first model to generate a prediction based on the accelerometer data, a second model to generate a prediction based on the image data, and so on). These modality-specific predictions may then be aggregated (e.g., summed, averaged, or used as input to a final scoring machine learning model to predict the action outcomes).

In some aspects, once the position machine learning model(s) 635 are trained, the training system 520 may deploy them for inferencing, along with the trained result machine learning model 640, as an ensemble outcome machine learning model 525. As discussed above, deploying the model(s) may generally include any operations used to prepare or provide the model to process input data (e.g., new sensor data) during runtime. For example, the training system 520 may instantiate the model locally, or may transmit the model details (e.g., the values of the learned parameters) to one or more other systems for inferencing.

In the illustrated example, the training data 620 includes a set of training records (also referred to in some aspects as exemplars, samples, result exemplars, or position exemplars), where each record comprises a set of one or more position label(s) 625 and a corresponding set of one or more outcome labels 515 (e.g., the outcome labels 515 of FIG. 5). As discussed above, the outcome labels 515 generally indicate the outcome(s) experienced by the patient after the user performed the action. In some aspects, the outcome labels 515 only indicate outcomes that were caused by (or are believed to have been caused by) the positioning of the patient, which was a result of the action performance itself. For example, pressure sores may be caused if the patient is left in the same position for a relatively long period of time. Accordingly, if the position labels 625 indicate and the user left the patient in the same body position, it may be inferred that the way the action was performed (moving the patient but allowing them to remain in the same orientation or seated position) caused the condition (pressure sores).

In the illustrated example, the training system 520 generates a result machine learning model 640 using the training data 620. Although not depicted in the illustrated example, in some aspects, one or more other data sources may also be used as input to the model during training. For example, in some embodiments, data relating to the patient(s) being assisted (e.g., from the patient database 130) may be used as input to the model. For example, information related to the patient's height, weight, age, ability to move independently, and the like may be useful inputs to predict results based on positioning.

Generally, the particular operations and techniques used to train the result machine learning model 640 may vary depending on the particular model architecture and implementation. For example, in the case of neural networks, the training system 520 may process the position labels 625 of a given sample of training data 620 as input to the result machine learning model 640 in order to generate an output prediction of results(s) that may result from the positioning (e.g., whether the patient will improve, worsen, or remain the same, whether the patient will contract any specific conditions, whether the patient will be comfortable, and the like). This prediction can then be compared against the corresponding outcome label 515 of the sample in order to generate a loss (e.g., using cross-entropy loss), and the loss can be used to update one or more model parameters (e.g., using backpropagation). This process may be performed iteratively for each training exemplar independently (e.g., using stochastic gradient descent) and/or in batches (e.g., using batch gradient descent).

Although a single result machine learning model 640 is depicted for conceptual clarity, in some embodiments, the training system 520 may train an ensemble of models. For example, in some embodiments, a different result machine learning model 640 for each outcome type (e.g., a first model trained to predict whether pressure sores will occur, a second model trained to predict whether bruising will occur, and the like).

In some embodiments, rather than using two separate models, the training system 520 may train the result machine learning model 640 to predict patient outcomes directly from the sensor data. That is, the sensor data (which indicates patient positioning) may be processed using the result machine learning model 640 to predict patient outcomes, in addition to or instead of processing the sensor data using a separate model to predict the patient positioning.

In some aspects, by using two separate models to predict user outcomes, the training system 520 may enable more granular and accurate predictions in some cases. For example, by first using the position machine learning model 635 to process sensor data in order to predict a position of the patient, and then using the result machine learning model to process the predicted positioning in order to predict patient outcome(s), the training system 520 decouples the predictions and allows each respective model to specialize on its own respective modality and task. This can result in substantially improved accuracy, in some implementations.

### Example Workflow to Refine Machine Learning Models to Predict Patient Outcomes for Specific Patients

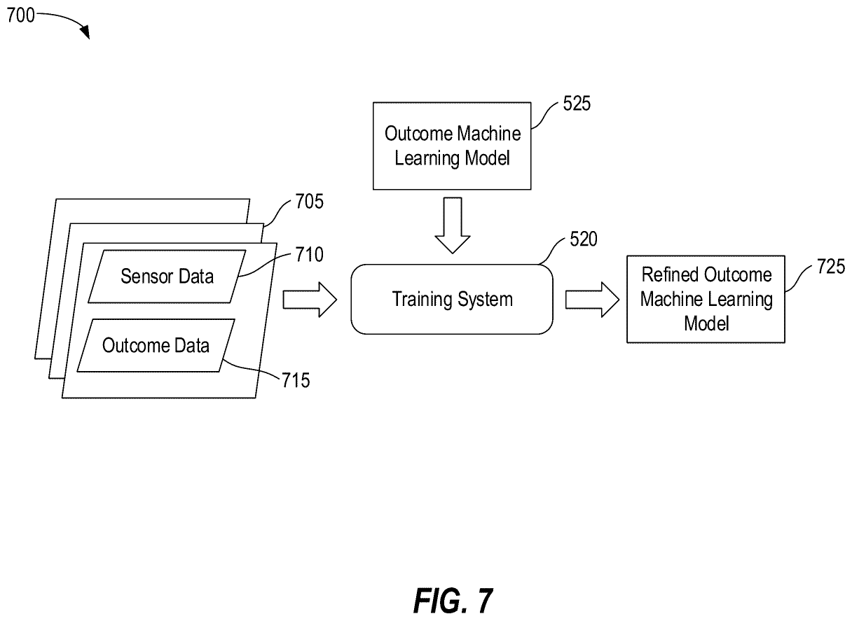

FIG. 7 depicts an example workflow 700 for refining machine learning models to predict patient outcomes for specific patients, according to some embodiments of the present disclosure.

In the illustrated example, a training system 520 (which may be the same as the training system 520 of FIG. 5 and/or FIG. 6, or may be a different system) accesses personalization data 705 to generate (e.g., train or refine) one or more refined outcome machine learning models 725.

As discussed above, though the training system 520 is depicted as a discrete system, some or all of the functionality of the training system 520 may be implemented across any number of systems, and the training system 520 may be implemented using hardware, software, or a combination of hardware and software. In the illustrated example, the training system 520 is configured to update or personalize machine learning models to evaluate sensor data for individual patients, as discussed in more detail below. In some embodiments, the training system 520 is implemented on the same system as the monitoring system 105 discussed above with reference to FIG. 1.

In the illustrated example, the personalization data 705 includes a set of training records (also referred to in some aspects as exemplars, samples, or personalization exemplars), where each record comprises sensor data 710, and outcome data 715. The sensor data 710 (which may correspond to the sensor data 120 of FIG. 1 and/or the sensor data 510 of FIG. 5) generally corresponds to data collected or measured using one or more sensors as a user (e.g., the user 110 of FIG. 1) performs an action (e.g., to assist a patient such as the patient 115 of FIG. 1). For example, as discussed above, the sensor data 710 may include acceleration and/or orientation data from one or more wearable sensors worn by the user, image data from one or more imaging sensors observing the user performing the action, and the like.

In the illustrated example, the sensor data 710 corresponds to a single patient. That is, while the sensor data 510 of FIG. 5 may depict action performance by any number of users for any number of patients (e.g., using a large number of samples from a large number of users to improve training and generalization of the models), the personalization data 705 may include exemplars of actions being performed for a single specific patient.

For example, to generate the personalization data 705, the training system 520 (or another system) may collect the sensor data 710 while one or more users perform actions for a given patient. In some embodiments, as discussed above, the sensor data 710 generally includes any data relevant to determining the potential outcomes of the particular action. For example, the orientation of the user's hands or the body of the patient may relevant for one action and/or outcome (e.g., to determine how the patient was positioned after the action was completed) and irrelevant for another. Similarly, the physical pressure exerted by the user may be relevant for some outcomes and irrelevant for others.

The outcome data 715 generally includes information relating to any outcome(s) that the specific patient experienced after the one or more action(s) were performed (e.g., improving, worsening, or remaining the same, contracting disorders, whether or not the patient was comfortable as a result of the action, and the like). As discussed above, some embodiments, the outcome data 715 is linked to corresponding sensor data 710 based on defined mappings. For example, some outcomes (e.g., pressure sores, bruising, broken bones, and the like) may be defined as only occurring (or only reasonably occurring) when one or more particular actions (e.g., transferring the patient) are performed incorrectly. In some embodiments, therefore, when such outcomes are noted, the training system 520 (or another system) may automatically evaluate patient data (e.g., from the patient database 130 of FIG. 1) to identify whether one or more of the corresponding action(s) were performed for the patient. In some aspects, this may include determining whether the action was performed within a timeline specified in the mapping (e.g., indicating the predicted or typical time that elapses between the action and the resulting outcome).

If such action(s) are found, the training system 520 (or another system) may automatically generate a new personalization exemplar that labels the corresponding sensor data 710 (collected when the action or actions were performed) with the noted outcome(s). In this way, the system can automatically build a set of personalization data 705 based on defined action-outcome mappings, and this personalization data 705 can be used to personalize the outcome machine learning models 525 to predict whether particular outcomes will occur for a specific patient.

In some embodiments, the personalization data 705 is collected while the outcome machine learning model 525 is being used to evaluate potential outcomes for the patient. For example, after being trained (e.g., using the workflow 500 of FIG. 5), the outcome machine learning model 525 may be used to predict outcomes while the user performs actions to assist the patient. In some aspects, in addition to evaluating the sensor data 710, the training system 520 (or another system) may store the sensor data 710 for future training or refinement of the outcome machine learning model 525. In some aspects, as the workflow 700 seeks to personalize the model for the specific patient, the personalization data 705 may lack labels at the time the sensor data 710 is captured. In some aspects, the personalization data 705 may be labeled with the corresponding outcomes subsequent to action performance. For example, during runtime, the training system 520 may collect feedback from the user, other experienced users observing the actions, the patient, and the like. In some embodiments, for example, if a patient suffers any negative outcomes (e.g., bruising, broken bones, pressure sores, and the like), these outcomes may be linked to the sensor data. This feedback may then be used to form labels for the sensor data 710.

In the illustrated example, the training system 520 generates a refined outcome machine learning model 725 by updating the outcome machine learning model 525 using the personalization data 705. Generally, the particular operations and techniques used to update or refine the outcome machine learning model 525 may vary depending on the particular model architecture and implementation, as discussed above. For example, in the case of neural networks, the training system 520 may process the sensor data 710 of a given sample of personalization data 705 as input to the outcome machine learning model 525 in order to generate an output prediction of outcomes. This prediction can then be compared against the corresponding label (e.g., against the outcome data 715 generated based on patient feedback or other patient data, as discussed above) of the sample in order to generate a loss (e.g., using cross-entropy loss), and the loss can be used to update one or more model parameters (e.g., using backpropagation). This process may be performed iteratively for each training exemplar independently (e.g., using stochastic gradient descent) and/or in batches (e.g., using batch gradient descent).

Although a single refined outcome machine learning model 725 is depicted for conceptual clarity, as discussed above, the training system 520 may generate an ensemble of personalized models in some embodiments. For example, in some embodiments, a different refined outcome machine learning model 725 may be refined to process each modality of sensor data 710, a different refined outcome machine learning model 725 may be trained for each action type and/or outcome type, and the like.

In some aspects, once the refined outcome machine learning model(s) 725 are trained, the training system 520 may deploy them for inferencing, as discussed above. For example, when processing future sensor data for actions done for the specific patient, the training system 520 may cease use of the non-personalized outcome machine learning model 525, and may instead process the new data using a refined outcome machine learning model 725 that was updated specifically for the specific patient based on outcome data 715 from the specific patient. This may improve results, such as when the model is personalized to predict whether the patient will be comfortable in a given position.

### Example Workflow to Use Machine Learning Models to Predict Patient Outcomes

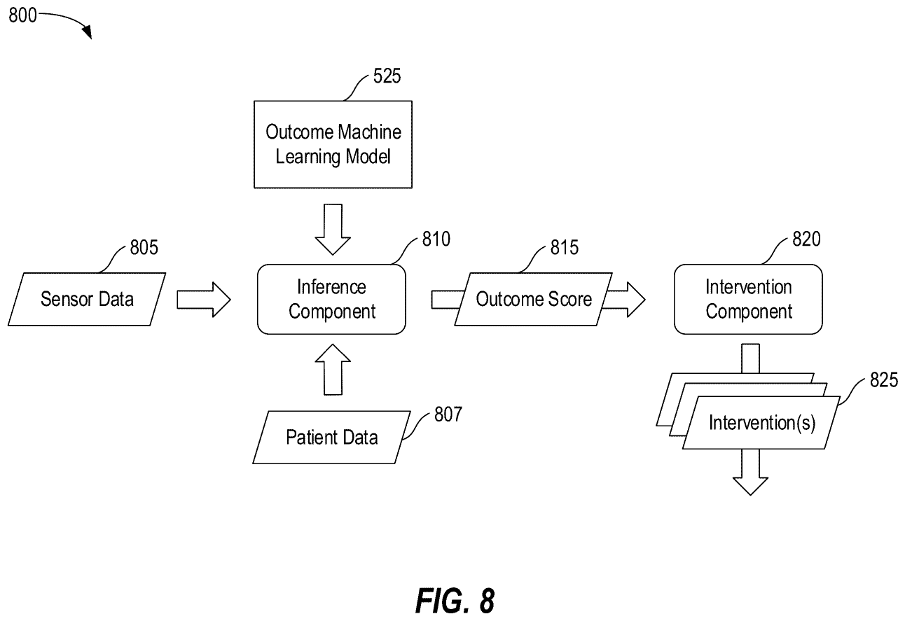

FIG. 8 depicts an example workflow 800 for using machine learning models to predict patient outcomes, according to some embodiments of the present disclosure.

In the illustrated example, an inference component 810 and an intervention component 820 accesses sensor data 805 and one or more trained outcome machine learning model(s) 525 to generate outcome score(s) 815 and intervention(s) 825. Though the inference component 810 and the intervention component 820 are depicted as a discrete components for conceptual clarity, in some embodiments some or all of the functionality of the depicted components may be implemented across any number of systems (e.g., a single system). Generally, the inference component 810 and intervention component 820 represent one or more computing systems (which may be implemented using hardware, software, or a combination of hardware and software) configured to use outcome machine learning models to evaluate sensor data and generate interventions, as discussed in more detail below. In some embodiments, the inference component 810 and the intervention component 820 are components of a monitoring system, such as the monitoring system 105 of FIG. 1, and/or of a training system, such as the training system 520 of FIG. 5. In some embodiments, the inference component 810 and the intervention component 820 correspond to the inference component 410 and the intervention component 420, each of FIG. 4, respectively.

In the illustrated example, the sensor data 805 (which may correspond to the sensor data 120 of FIG. 1 and/or the sensor data 510 of FIG. 5) generally corresponds to data collected or measured using one or more sensors as a user (e.g., the user 110 of FIG. 1) performs an action (e.g., to assist a patient such as the patient 115 of FIG. 1). For example, as discussed above, the sensor data 805 may include acceleration and/or orientation data from one or more wearable sensors worn by the user, image data from one or more imaging sensors observing the user performing the action, and the like.

In the illustrated example, the inference component 810 accesses the sensor data 805 and patient data 807, and processes it using an outcome machine learning model 525 to generate an outcome score 815. Generally, the patient data 807 is representative of any patient characteristics that may be relevant to predicting outcomes for the specific patient, as discussed above. For example, the patient data 807 may include information such as the patient's age, gender, height, weight, previous injuries or outcomes, comorbidities, and the like.

Although a single outcome machine learning model 525 is depicted for conceptual clarity, in some embodiments, the inference component 810 may use an ensemble of models (e.g., processing each modality of sensor data 805 using a different model, processing the sensor data 805 using a different model (or set of models) based on the action being performed, and the like). Further, although not included in the illustrated example, in some embodiments the inference component 810 may process the sensor data 805 using a personalized or customized model (e.g., the refined outcome machine learning model 725 of FIG. 7) that has been personalized for the specific patient for whom the action is being performed.

In some aspects, as discussed above, the inference component 810 accesses the sensor data 805 in response to one or more triggers, such as determining that the user is about to perform (or is performing) the action. For example, as discussed above, the user may press a button on their smartphone or a wearable device, verbally announce the action, and the like. In response, the inference component 810 may begin accessing the sensor data 805 (e.g., by activating one or more sensors in the room). In some embodiments, once the inference component 810 determines that the action is complete (e.g., when the user presses a button again, verbally announces completion, steps away from the patient, and the like), the inference component 810 can similarly deactivate the sensor(s).

Although not depicted for conceptual clarity, in some aspects, the inference component 810 may perform one or more preprocessing steps on the sensor data 805 to prepare the data to be used as input to the outcome machine learning model 525, as discussed above.

The outcome score 815 is generally representative of a prediction, generated by the outcome machine learning model 525, indicating the potential outcome(s) that the performance of the action may cause to the patient. In some embodiments, the outcome score 815 is a categorical score (e.g., a binary value indicating whether the performance will cause one or more outcomes, such as whether the patient will be comfortable or whether they will contract a new medical condition). In some embodiments, the outcome score 815 is (or includes) a continuous value or measure (e.g., scoring the probability of one or more outcomes between zero and one, where one represents near certainty of an outcome occurring).

In some embodiments, as discussed above, the outcome score 815 may be stored in a repository (such as the patient database 130 of FIG. 1), or may otherwise be used to update stored data for the patient.

In the illustrated workflow 800, the outcome score 815 is accessed by the intervention component 820. The intervention component 820 generally evaluates the outcome score 815 using one or more criteria (e.g., rules and/or thresholds) to determine what intervention(s) 825, if any, to initiate. For example, in some embodiments, the intervention component 820 may determine whether the outcome score 815 is above or below a defined threshold. If the outcome score 815 is above a threshold (e.g., indicating a relatively high chance of the outcome), the intervention component 820 may determine to initiate one or more interventions 825. As discussed above, in some embodiments, the intervention component 820 may use multiple thresholds or rules to evaluate the outcome score 815 and determine which intervention(s) 825 should be used.

For example, in some embodiments, the intervention component 820 may generate and transmit a notification to the user depicted by the sensor data 805, may transmit instructional or tutorial information to the user to assist them in improving, may generate and transmit a notification to the supervisor or manager of the user, and the like.

In some embodiments, the interventions 825 may generally indicate relevant information such as the user that was performing the action, the action that was being performed, the date and/or time when the action was performed, the patient that was being assisted, the potential outcomes that are at risk, and the like. In some embodiments, the notification may indicate the outcome score 815 itself.

In some embodiments, in addition to determining which (if any) interventions 825 to initiate, the intervention component 820 may also determine which samples of sensor data 805 (if any) to save for future refinement of the outcome machine learning model 525 (e.g., to generate a personalized model using the workflow 700 of FIG. 7). For example, the intervention component 820 may determine whether the outcome score 815 meets or exceeds a threshold. If so (e.g., if the action results in a substantial chance of negative outcomes), the intervention component 820 may save the sensor data 805 to personalize or refine the model specifically for the given patient and/or other patients.

Generally, the workflow 800 may be used whenever sensor data 805 is available and/or whenever the user performs an action, as discussed above. This can ensure reliable monitoring of the patient outcomes and improve the quality of assistance offered to patients.

### Example Workflow to Train Machine Learning Models to Predict Action Injury Risk

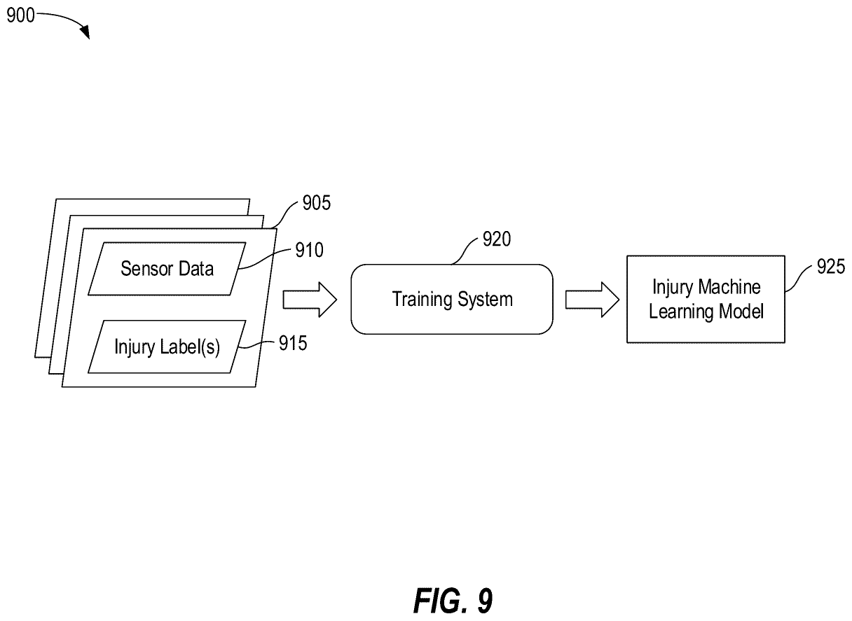

FIG. 9 depicts an example workflow 900 for training machine learning models to predict action injury risk, according to some embodiments of the present disclosure.

In the illustrated example, a training system 920 accesses training data 905 to generate (e.g., train) one or more injury machine learning models 925. Though the training system 920 is depicted as a discrete system, in some embodiments some or all of the functionality of the training system 920 may be implemented across any number of systems. Generally, the training system 920 represents a computing system (which may be implemented using hardware, software, or a combination of hardware and software) configured to train and/or update machine learning models to evaluate sensor data, as discussed in more detail below. In some embodiments, the training system 920 is implemented on the same system as the monitoring system 105 discussed above with reference to FIG. 1, the training system 220 discussed above with reference to FIGS. 2-3, and/or the training system 520 discussed above with reference to FIGS. 5-7.

In the illustrated example, the training data 905 includes a set of training records (also referred to in some aspects as exemplars, samples, action exemplars, or injury exemplars), where each record comprises sensor data 910 and a corresponding set of one or more injury labels 915. The sensor data 910 (which may correspond to the sensor data 120 of FIG. 1, the sensor data 210 of FIG. 2, and/or the sensor data 510 of FIG. 5) generally corresponds to data collected or measured using one or more sensors as a user (e.g., the user 110 of FIG. 1) performs an action (e.g., to assist a patient such as the patient 115 of FIG. 1). For example, as discussed above, the sensor data 910 may include acceleration and/or orientation data from one or more wearable sensors worn by the user, image data from one or more imaging sensors observing the user performing the action, and the like.

In the illustrated example, the injury label(s) 915 generally indicate the injury (or injuries)) experienced by the user as a result of performing the action. For example, the injury label 915 may indicate whether the user suffered any condition or disorder such as a sprained wrist, injured back, and the like. In some aspects, the injury labels 915 only indicate injuries that were caused by (or are believed to have been caused by) the action performance itself. For example, a strained back may be caused by lifting the patient. Accordingly, if the action involved helping the patient move or transfer between chairs and/or beds, and the user reports having a strained back, it may be inferred that the way the action was performed (e.g., how the patient was lifted) caused the injury (a strained back). Accordingly, an injury label 915 may be generated indicating that the action performance (as reflected in the sensor data 910) caused the indicated injury (e.g., bruising).

In some aspects, the injury labels 915 comprise one or more continuous values (e.g., a score between zero and one) indicating the severity of the injury (e.g., where large values indicate substantially negative outcomes, such as higher values for worse injuries and/or higher values for worse states of the same injury, such as worse sprains). In some embodiments, the injury labels 915 comprise one or more binary values (e.g., indicating whether each injury occurred).

As discussed above, in some embodiments, the injury labels 915 are determined based on defined mappings. For example, some injuries may be defined as only occurring (or reasonably occurring) when one or more particular actions (e.g., transferring the patient) are performed incorrectly. In some embodiments, therefore, when such injuries are noted, the training system 920 (or another system) may automatically evaluate patient data (e.g., from the patient database 130 of FIG. 1) to identify whether the user recently performed one or more of the corresponding action(s) for a patient. In some aspects, this may include determining whether the action was performed within a timeline specified in the mapping (e.g., indicating the predicted or typical time that elapses between the action and the resulting injury).

If such action(s) are found, the training system 920 (or another system) may automatically generate a training exemplar that labels the corresponding sensor data 910 (collected when the action or actions were performed) with the noted injury (or injuries). In this way, the system can automatically build a set of training data 905 based on defined action-injury mappings, and this training data 905 can be used to train the injury machine learning models 925 to predict whether users will suffer particular injuries based on how they perform action(s) (e.g., based on sensor data collected while users perform the actions). For example, the model may predict that if a user uses a certain technique to assist the patient, they are likely to injure their wrist (either immediately, or at some future point after repeated performance of the action).

In some embodiments, the injury labels 915 are additionally or alternatively defined based at least in part on manual review by experts or experienced users. For example, an experienced user (e.g., a supervisor) may observe another user performing an action (either in real-time or subsequently), such as by reviewing video or imagery of the action, reviewing sensor data such as pressure information, and the like. The experienced user may then indicate what injuries, if any, they predict the user will suffer (e.g., while correcting the user if the action is being performed in real-time). In some embodiments, some or all of the training data 905 may be generated based on such manual evaluation and correction.

As another example, in some embodiments, the injury labels 915 are additionally or alternatively generated based at least in part on subjective feedback (e.g., from the user who the action). For example, to generate the training data 905, the training system 920 (or another system) may collect the sensor data 910 while a user performs an action, and then generate an injury label 915 to score the injury risk (e.g., by surveying the user about any concerns they have regarding the action, such as soreness, strain, and the like). Generally, the injury labels 915 (and the particular modalities reflected in the sensor data 910) may vary depending on the particular implementation and action type. The sensor data 910 generally includes any data relevant to determining the potential injury risk, and the injury labels 915 may be defined using any criteria relevant to the particular injuries and actions. For example, the orientation of the user's hands may relevant for one action (e.g., to determine whether they will strain their wrist) and irrelevant for another.

Although not depicted in the illustrated example, in some aspects, one or more other data sources may also be used as input to the model during training. For example, in some embodiments, data relating to the user themselves (e.g., from the user database 125) may be used as input. For example, the user's height, weight, strength, age, previous injuries, and the like may be relevant inputs to the model. Similarly, in some embodiments, data relating to the patient(s) being assisted (e.g., from the patient database 130) may be used as input to the model. For example, information related to the patient's height, weight, age, and the like may be useful inputs to predict injury risk.

In the illustrated example, the training system 920 generates an injury machine learning model 925 using the training data 905. Generally, the particular operations and techniques used to train the injury machine learning model 925 may vary depending on the particular model architecture and implementation. For example, in the case of neural networks, the training system 920 may process the sensor data 910 of a given sample of training data 905 as input to the injury machine learning model 925 in order to generate an output prediction of injuries that may result from the performance. This prediction can then be compared against the corresponding injury label 915 of the sample in order to generate a loss (e.g., using cross-entropy loss), and the loss can be used to update one or more model parameters (e.g., using backpropagation). This process may be performed iteratively for each training exemplar independently (e.g., using stochastic gradient descent) and/or in batches (e.g., using batch gradient descent).

Although a single injury machine learning model 925 is depicted for conceptual clarity, in some embodiments, the training system 920 may train an ensemble of models. For example, in some embodiments, a different injury machine learning model 925 may be trained to process each modality of sensor data 910 (e.g., a first model to generate a prediction based on the accelerometer data, a second model to generate a prediction based on the image data, and so on). These modality-specific predictions may then be aggregated (e.g., summed, averaged, or used as input to a final scoring machine learning model to predict the injury risks).

As another example, in some embodiments, the training system 920 may train a different injury machine learning model 925 for each injury type (e.g., a first model trained to predict whether the user will injure their wrist, a second model trained to predict whether the user will injure their back, and the like). As yet another example, in some embodiments, the training system 920 may train a different injury machine learning model 925 for each action type. For example, a first model may be trained to predict injuries that may occur as a result of performance of a first action (e.g., lifting a patient) while a second model may be trained to predict the potential injuries of a different action (e.g., transferring the patient), a third model may be trained to predict the injuries resulting from a third action (e.g., bathing the patient), and so on.

In some aspects, once the injury machine learning model(s) 925 are trained, the training system 920 may deploy them for inferencing. As discussed above, deploying the model may generally include any operations used to prepare or provide the model to process input data (e.g., new sensor data) during runtime. For example, the training system 920 may instantiate the model locally, or may transmit the model details (e.g., the values of the learned parameters) to one or more other systems for inferencing.

### Example Workflow to Refine Machine Learning Models to Predict Action Injury Risk for Specific Users

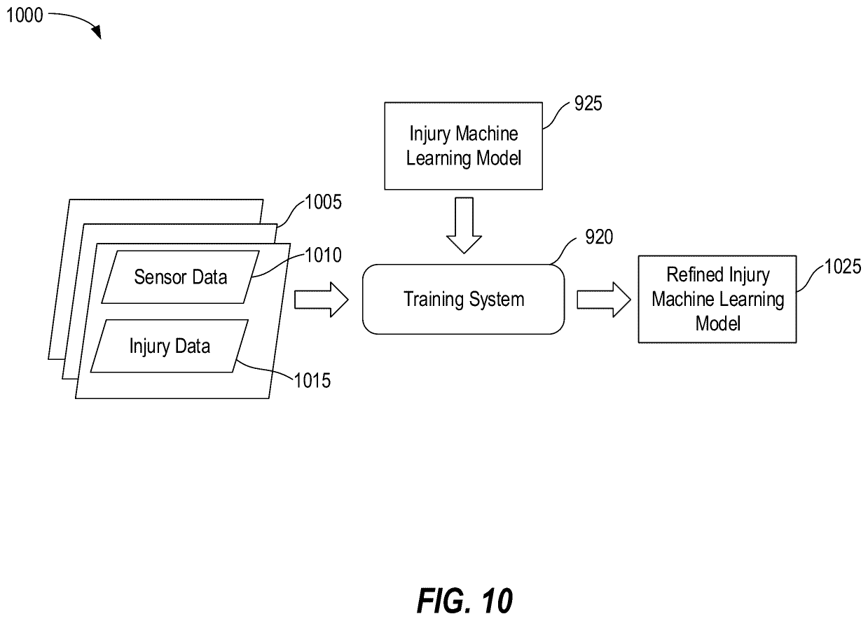

FIG. 10 depicts an example workflow 1000 for refining machine learning models to predict action injury risk for specific users, according to some embodiments of the present disclosure.

In the illustrated example, a training system 920 (which may be the same as the training system 920 of FIG. 9, or may be a different system) accesses personalization data 1005 to generate (e.g., train or refine) one or more refined injury machine learning models 1025. As discussed above, though the training system 920 is depicted as a discrete system, some or all of the functionality of the training system 920 may be implemented across any number of systems, and the training system 920 may be implemented using hardware, software, or a combination of hardware and software. In the illustrated example, the training system 920 is configured to update or personalize machine learning models to evaluate sensor data for individual users, as discussed in more detail below. In some embodiments, the training system 920 is implemented on the same system as the monitoring system 105 discussed above with reference to FIG. 1.

In the illustrated example, the personalization data 1005 includes a set of training records (also referred to in some aspects as exemplars, samples, or personalization exemplars), where each record comprises sensor data 1010 and injury data 1015. The sensor data 1010 (which may correspond to the sensor data 120 of FIG. 1 and/or the sensor data 910 of FIG. 9) generally corresponds to data collected or measured using one or more sensors as a user (e.g., the user 110 of FIG. 1) performs an action (e.g., to assist a patient such as the patient 115 of FIG. 1). For example, as discussed above, the sensor data 1010 may include acceleration and/or orientation data from one or more wearable sensors worn by the user, image data from one or more imaging sensors observing the user performing the action, and the like.

In the illustrated example, the sensor data 1010 corresponds to a single user. That is, while the sensor data 910 of FIG. 9 may depict action performance by any number of users for any number of patients (e.g., using a large number of samples from a large number of users to improve training and generalization of the models), the personalization data 1005 may include exemplars of actions being performed by a single specific user.

For example, to generate the personalization data 1005, the training system 920 (or another system) may collect the sensor data 910 while the specific user performs actions for one or more patients. In some embodiments, as discussed above, the sensor data 1010 generally includes any data relevant to determining the potential injury risks associated with the particular action. For example, the orientation of the user's hands or the body of the patient may relevant for one action and/or injury (e.g., to determine whether the user will strain their wrist) and irrelevant for another.

The injury data 1015 generally includes information relating to any injuries that the specific user experienced after or during performance of one or more action(s). As discussed above, some embodiments, the injury data 1015 is linked to corresponding sensor data 1010 based on defined mappings. For example, some injuries (e.g., sprained wrists) may be defined as only occurring (or only reasonably occurring) when one or more particular actions (e.g., inserting a catheter) are performed incorrectly. In some embodiments, therefore, when such outcomes are noted, the training system 920 (or another system) may automatically evaluate patient data (e.g., from the patient database 130 of FIG. 1) and/or user data (e.g., from the user database 125 of FIG. 1) to identify whether the user performed one or more of the corresponding action(s). In some aspects, this may include determining whether the action was performed within a timeline specified in the mapping (e.g., indicating the predicted or typical time that elapses between the action and the resulting outcome).

If such action(s) are found, the training system 920 (or another system) may automatically generate a new personalization exemplar that labels the corresponding sensor data 1010 (collected when the action or actions were performed) with the noted outcome(s). In this way, the system can automatically build a set of personalization data 1005 based on defined action-outcome mappings, and this personalization data 1005 can be used to personalize the injury machine learning models 925 to predict whether particular injuries will occur for a specific user.

In some embodiments, the personalization data 1005 is collected while the injury machine learning model 925 is being used to evaluate potential injuries for the user. For example, after being trained (e.g., using the workflow 900 of FIG. 9), the injury machine learning model 925 may be used to predict injury risks while the user performs actions to assist patients. In some aspects, in addition to evaluating the sensor data 1010, the training system 920 (or another system) may store the sensor data 1010 for future training or refinement of the injury machine learning model 925.

In some aspects, as the workflow 1000 seeks to personalize the model for the specific user, the personalization data 1005 may lack labels at the time the sensor data 1010 is captured. In some aspects, the personalization data 1005 may be labeled with the corresponding injuries subsequent to action performance. For example, during runtime, the training system 920 may collect feedback from the user, other experienced users observing the actions, and the like. In some embodiments, for example, if a user suffers an injury, these injuries may be linked to the sensor data. This feedback may then be used to form labels for the sensor data 1010.

In the illustrated example, the training system 920 generates a refined injury machine learning model 1025 by updating the injury machine learning model 925 using the personalization data 1005. Generally, the particular operations and techniques used to update or refine the injury machine learning model 925 may vary depending on the particular model architecture and implementation, as discussed above. For example, in the case of neural networks, the training system 920 may process the sensor data 1010 of a given sample of personalization data 1005 as input to the injury machine learning model 925 in order to generate an output prediction of injury risks. This prediction can then be compared against the corresponding label (e.g., against the injury data 1015 generated based on user feedback, as discussed above) of the sample in order to generate a loss (e.g., using cross-entropy loss), and the loss can be used to update one or more model parameters (e.g., using backpropagation). This process may be performed iteratively for each training exemplar independently (e.g., using stochastic gradient descent) and/or in batches (e.g., using batch gradient descent).

Although a single refined injury machine learning model 1025 is depicted for conceptual clarity, as discussed above, the training system 920 may generate an ensemble of personalized models in some embodiments. For example, in some embodiments, a different refined injury machine learning model 1025 may be refined to process each modality of sensor data 1010, a different refined injury machine learning model 1025 may be trained for each action type and/or injury type, and the like.

In some aspects, once the refined injury machine learning model(s) 1025 are trained, the training system 920 may deploy them for inferencing, as discussed above. For example, when processing future sensor data for actions done for the specific user, the training system 920 may cease use of the non-personalized injury machine learning model 925, and may instead process the new data using a refined injury machine learning model 1025 that was updated specifically for the specific user based on injury data 1015 from the specific user.

### Example Workflow to Use Machine Learning Models to Predict Action Injury Risk

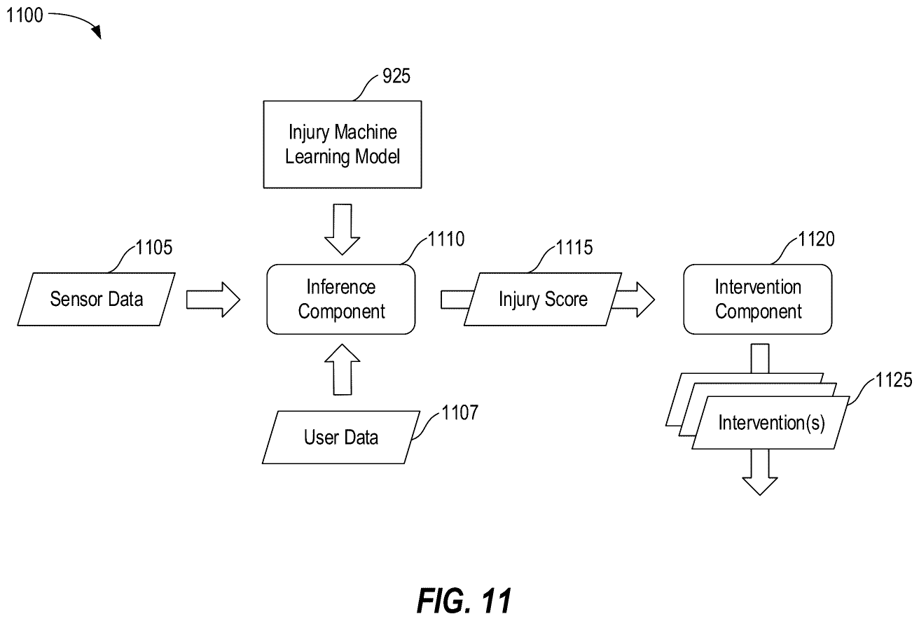

FIG. 11 depicts an example workflow 1100 for using machine learning models to predict action injury risk, according to some embodiments of the present disclosure.

In the illustrated example, an inference component 1110 and an intervention component 1120 accesses sensor data 1105 and one or more trained injury machine learning model(s) 925 to generate injury score(s) 1115 and intervention(s) 1125. Though the inference component 1110 and the intervention component 1120 are depicted as a discrete components for conceptual clarity, in some embodiments some or all of the functionality of the depicted components may be implemented across any number of systems (e.g., a single system). Generally, the inference component 1110 and intervention component 1120 represent one or more computing systems (which may be implemented using hardware, software, or a combination of hardware and software) configured to use outcome machine learning models to evaluate sensor data and generate interventions, as discussed in more detail below. In some embodiments, the inference component 1110 and the intervention component 1120 are components of a monitoring system, such as the monitoring system 105 of FIG. 1, and/or of a training system, such as the training system 920 of FIG. 9. In some embodiments, the inference component 1110 and the intervention component 1120 correspond to the inference component 410 and the intervention component 420, each of FIG. 4, respectively, and/or to the inference component 810 and the intervention component 820, each of FIG. 8, respectively.

In the illustrated example, the sensor data 1105 (which may correspond to the sensor data 120 of FIG. 1 and/or the sensor data 910 of FIG. 9) generally corresponds to data collected or measured using one or more sensors as a user (e.g., the user 110 of FIG. 1) performs an action (e.g., to assist a patient such as the patient 115 of FIG. 1). For example, as discussed above, the sensor data 1105 may include acceleration and/or orientation data from one or more wearable sensors worn by the user, image data from one or more imaging sensors observing the user performing the action, and the like.

In the illustrated example, the inference component 1110 accesses the sensor data 1105 and user data 1107, and processes it using an injury machine learning model 925 to generate an injury score 1115. Generally, the user data 1107 is representative of any user characteristics that may be relevant to predicting injury risks for the specific user, as discussed above. For example, the user data 1107 may include information such as the user's age, gender, height, weight, previous injuries, and the like.

Although a single injury machine learning model 925 is depicted for conceptual clarity, in some embodiments, the inference component 1110 may use an ensemble of models (e.g., processing each modality of sensor data 1105 using a different model, processing the sensor data 1105 using a different model (or set of models) based on the action being performed, and the like). Further, although not included in the illustrated example, in some embodiments the inference component 1110 may process the sensor data 1105 using a personalized or customized model (e.g., the refined injury machine learning model 1025 of FIG. 10) that has been personalized for the specific user performing the action.

In some aspects, as discussed above, the inference component 1110 accesses the sensor data 1105 in response to one or more triggers, such as determining that the user is about to perform (or is performing) the action. For example, as discussed above, the user may press a button on their smartphone or a wearable device, verbally announce the action, and the like. In response, the inference component 1110 may begin accessing the sensor data 1105 (e.g., by activating one or more sensors in the room). In some embodiments, once the inference component 1110 determines that the action is complete (e.g., when the user presses a button again, verbally announces completion, steps away from the patient, and the like), the inference component 1110 can similarly deactivate the sensor(s).

Although not depicted for conceptual clarity, in some aspects, the inference component 1110 may perform one or more preprocessing steps on the sensor data 1105 to prepare the data to be used as input to the injury machine learning model 925, as discussed above.

The injury score 1115 is generally representative of a prediction, generated by the injury machine learning model 925, indicating the potential injuries that the performance of the action may cause to the user. In some embodiments, the injury score 1115 is a categorical score (e.g., a binary value indicating whether the performance will cause one or more injuries). In some embodiments, the injury score 1115 is (or includes) a continuous value or measure (e.g., scoring the probability of one or more injuries between zero and one, where one represents near certainty of an injury occurring).

In some embodiments, as discussed above, the injury score 1115 may be stored in a repository (such as the user database 125 of FIG. 1), or may otherwise be used to update stored data for the user.

In the illustrated workflow 1100, the injury score 1115 is accessed by the intervention component 1120. The intervention component 1120 generally evaluates the injury score 1115 using one or more criteria (e.g., rules and/or thresholds) to determine what intervention(s) 1125, if any, to initiate. For example, in some embodiments, the intervention component 1120 may determine whether the injury score 1115 is above or below a defined threshold. If the injury score 1115 is above a threshold (e.g., indicating a relatively high chance of the injury), the intervention component 1120 may determine to initiate one or more interventions 1125. As discussed above, in some embodiments, the intervention component 1120 may use multiple thresholds or rules to evaluate the injury score 1115 and determine which intervention(s) 1125 should be used.

For example, in some embodiments, the intervention component 1120 may generate and transmit a notification to the user depicted by the sensor data 1105, may transmit instructional or tutorial information to the user to assist them in improving, may generate and transmit a notification to the supervisor or manager of the user, may reassign the user to perform different assistance actions and/or assign a different user to assist the current patient, and the like.

In some embodiments, the interventions 1125 may generally indicate relevant information such as the user that was performing the action, the action that was being performed, the date and/or time when the action was performed, the patient that was being assisted, the potential injuries that are at risk, and the like. In some embodiments, the notification may indicate the injury score 1115 itself.

In some embodiments, in addition to determining which (if any) interventions 1125 to initiate, the intervention component 1120 may also determine which samples of sensor data 1105 (if any) to save for future refinement of the injury machine learning model 925 (e.g., to generate a personalized model using the workflow 1000 of FIG. 10). For example, the intervention component 1120 may determine whether the injury score 1115 meets or exceeds a threshold. If so (e.g., if the action results in a substantial chance of injury), the intervention component 1120 may save the sensor data 1105 to personalize or refine the model specifically for the given user and/or other users.

Generally, the workflow 1100 may be used whenever sensor data 1105 is available and/or whenever the user performs an action, as discussed above. This can ensure reliable monitoring of the patient outcomes and improve the quality of assistance offered to patients.

### Example Method for Training Machine Learning Models to Predict Action Performance Quality

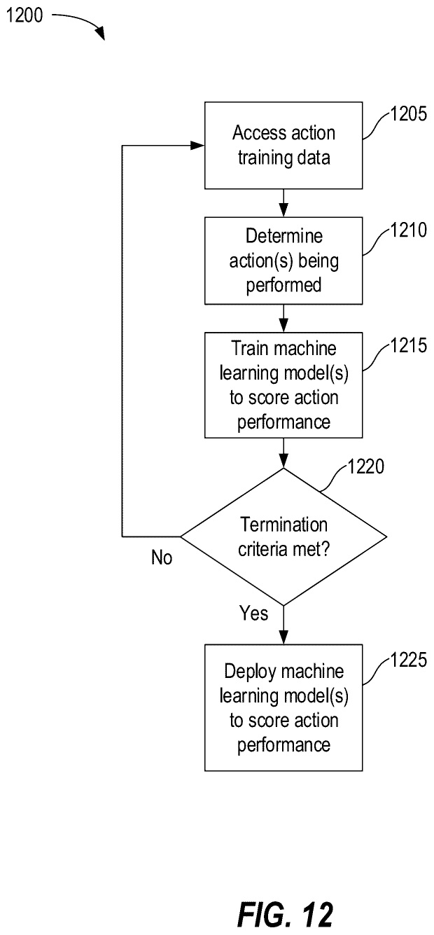

FIG. 12 is a flow diagram depicting an example method 1200 for training machine learning models to predict action performance quality, according to some embodiments of the present disclosure. In some aspects, the method 1200 may be performed by a training system, such as the training system 220 of FIGS. 2-3, the training system 520 of FIGS. 5-7, the training system 920 of FIGS. 9-10, and the like. In some aspects, the method 1200 provides additional detail for the workflow 200 of FIG. 2.

At block 1205, the training system accesses action training data. Generally, the action training data comprises information related to actions performed by one or more users to assist one or more patients. For example, the action training data may correspond to the training data 205 of FIG. 2. In some embodiments, as discussed above, the action training data may include a set of training samples or exemplars, where each sample includes sensor data depicting or indicative of the action performance (e.g., the sensor data 210 of FIG. 2), as well as a label indicating the quality of the performed action (e.g., the action labels 215 of FIG. 2).

As discussed above, the sensor data may generally include any relevant data, collected via any suitable sensor or device, which may be useful in identifying action(s) being performed and/or in quantifying how well the action was performed. For example, the sensor data may include data from one or more wearable sensors on the user and/or patient (e.g., acceleration information, orientation information, pressure information, and the like), data from one or more sensors in the physical space or room of the patient (e.g., pressure sensors, cameras, microphones, and the like), and any other relevant data.

The action labels are generally indicative of how well the action was performed. As discussed above, this action quality may be a quantitative value indicating, for example, how closely the user's movements mirrored the preferred or instructed technique for performing an action. For example, the label may include a score between 0 and 1 indicating how closely the user's acceleration matched the preferred acceleration, how closely the user's hand movements matched the instructed technique, and the like. In some aspects, as discussed above, the training data may be generated while an instructor or experienced user performs tasks according to the designated techniques.

At block 1210, the training system determines which action(s) were being performed for one or more of the exemplars of training data. For example, as discussed above, the training system may infer the action based on the movements or sensor data itself, or may identify the action based on manual or explicit labeling. For example, in some embodiments, the user may announce or record what actions they are performing (or did perform) to assist a patient. The training system may use this information to determine which action(s) each sample of training data correspond to.

At block 1215, the training system trains one or more machine learning models (e.g., the quality machine learning model 225 of FIG. 2) based on the training data. Generally, as discussed above, the particular operations and techniques used to train the machine learning model(s) may vary depending on the particular model architecture and implementation. For example, in the case of neural networks, the training system may process the sensor data of one or more samples of training data as input to the machine learning model(s) to generate an output prediction of performance quality. This prediction can then be compared against the corresponding label(s) of one or more samples in order to generate a loss (e.g., using cross-entropy loss), and the loss can be used to update one or more model parameters (e.g., using backpropagation). This process may be performed iteratively for each training exemplar independently (e.g., using stochastic gradient descent) and/or in batches (e.g., using batch gradient descent).

In this way, the model can iteratively learn to predict the quality of action performance based on the sensor data collected while the user performs the action.

At block 1220, the training system determines whether one or more training termination criteria have been met. The termination criteria may generally vary depending on the particular implementation. For example, in some embodiments, the training system may determine whether a defined period of time and/or amount of computing resources have been spent training the model(s). In some embodiments, the training system may determine whether the model(s) have reached a defined or preferred minimum accuracy. In some embodiments, the training system may determine whether a defined number of training iterations or epochs have been completed. In some embodiments, the training system may determine whether any additional training samples remain to be used.

If the training system determines that the termination criteria are not met, the method 1200 returns to block 1205 to access more (or new) training data. If, at block 1220, the training system determines that the termination criteria are met, the method 1200 continues to block 1225, where the training system deploys the machine learning model(s) to score action performance during runtime. As discussed above, deploying the model(s) may generally include any operations used to prepare or provide the model to process input data (e.g., new sensor data) during runtime. For example, the training system may instantiate the model locally, or may transmit the model details (e.g., the values of the learned parameters) to one or more other systems for inferencing.

### Example Method for Generating Training Data for Machine Learning Models to Predict Action Performance Quality

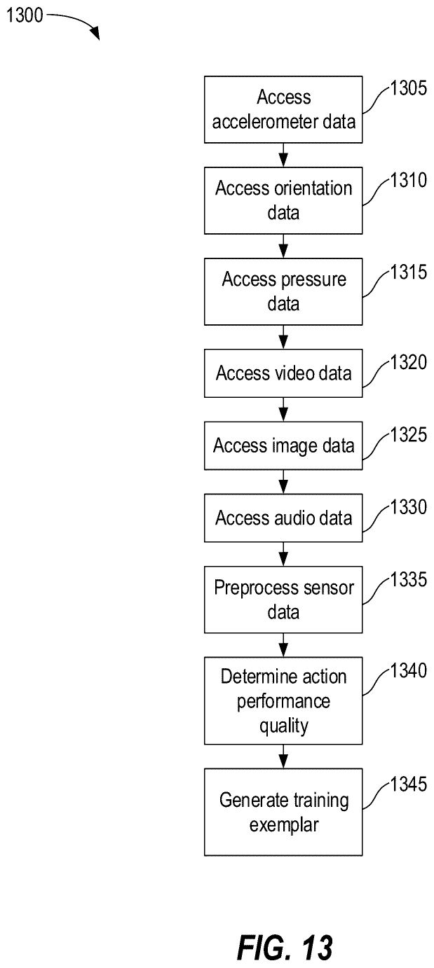

FIG. 13 is a flow diagram depicting an example method 1300 for generating training data for machine learning models to predict action performance quality, according to some embodiments of the present disclosure. In some aspects, the method 1300 may be performed by a training system, such as the training system 220 of FIGS. 2-3, the training system 520 of FIGS. 5-7, the training system 920 of FIGS. 9-10, and/or the training system discussed above with reference to FIG. 12. In some aspects, the method 1300 provides additional detail for block 1205 of FIG. 12.

At block 1305, the training system accesses accelerometer data collected via one or more sensors as a user performs a task for a patient. For example, as discussed above, the accelerometer data may be collected via accelerometers embedded in one or more wearable devices, such as on the user's wrist(s), on the patient, and the like. In some aspects, the accelerometer data is collected from other devices or components, such as a smartphone of the user and/or patient, or one or more tools or devices used to provide the assistance (e.g., an accelerometer in a walker or cane used to help the patient walk).

At block 1310, the training system accesses orientation data collected via one or more orientation sensors as the user performs the task. In some embodiments, the orientation data may be collected from similar (or the same) devices to the accelerometer data. For example, the orientation data may be collected via orientation sensors embedded in one or more wearable devices, such as on the user's wrist(s), on the patient, and the like. In some aspects, the orientation data is collected from other devices or components, such as a smartphone of the user and/or patient, or one or more tools or devices used to provide the assistance (e.g., an orientation sensor in a walker or cane used to help the patient walk).

At block 1315, the training system accesses pressure data collected via one or more sensors during or after the user performs the task. For example, as discussed above the pressure data may be collected via pressure sensors to indicate information such as the grip strength of the user, the positioning of the patient, the amount of pressure exerted on the patient by the user, and the like.

At block 1320, the training system accesses video data depicting the user performing the action for the patient. For example, as discussed above, one or more video cameras may capture the performance from one or more angles in the space. In some embodiments, the video can include multiple types of video, such as color video for visible light (e.g., red, green, and blue (RGB)) video, video with depth information (e.g., RGB-Depth (RBG-D)) video, stereoscopic video, thermal video, and the like. In some embodiments, the user and/or patient may be prompted to capture the video data during or after performance of the action (e.g., to confirm that it was completed).

At block 1325, the training system accesses image data depicting the user performing the action for the patient. In some embodiments, the image data may be collected if video data is not available (e.g., due to insufficient bandwidth to transmit full video data, or because the room does not contain a video camera). For example, a video may be captured, and a subset of the frames from the video may be accessed as training data. For example, as discussed above, one or more video cameras may capture the performance from one or more angles in the space. In some embodiments, the image data can include multiple types of images, such as color images in the visible light spectrum, images with depth information, stereoscopic images, thermal images, and the like. In some embodiments, the user and/or patient may be prompted to capture the image data during or after performance of the action (e.g., to confirm that it was completed).

At block 1330, the training system accesses audio data of the user performing the action for the patient. For example, as discussed above, one or more microphones may capture the performance from one or more positions in the space. In some embodiments, the audio data includes audio in the audible frequency range. Generally, the audio data may include natural language speech (e.g., of the user and/or patient) as well as non-language audio (e.g., sounds caused by the action performance, such as a shower running, a pot boiling, a vacuum running, a toilet flushing, and the like). In some embodiments, the user and/or patient may be prompted to capture the audio data during or after performance of the action (e.g., to confirm that it was completed).

Although a number of data modalities (e.g., including accelerometer, orientation, pressure, video, image, and audio data) are depicted for conceptual clarity, these modalities are intended to be used as examples, and are not limiting on embodiments of the present disclosure. That is, the training system may use more or fewer modalities than those contemplated herein, and may generally use any relevant or useful sensor data.

At block 1335, the training system optionally preprocesses the sensor data if appropriate to render the data more suited for processing using machine learning models. Generally, the particular operations used for preprocessing may vary depending on the particular implementation and modality of the data. For example, the training system may remove outlier data, denoise the data, delineate the data into windows or points in time, aggregate the data, and the like.

At block 1340, the training system determines the quality of the action performance. That is, the training system determines how well the user completed the action. In some aspects, as discussed above, this performance quality is provided by the user, the patient, or another. For example, if the user is an experienced or expert caregiver who is intentionally recording training data for the training system, the training system may determine that the action was performed well or in accordance with the desired techniques. Generally, a wide variety of operations may be used to determine or infer the action quality, as discussed above.

At block 1345, the training system generates a training exemplar including the accessed sensor data (e.g., from each modality used) as the input portion of the exemplar, as well as the determined performance quality as the target output or label of the exemplar. In this way, the training exemplar may be stored and/or used to train or refine one or more quality machine learning models, as discussed above. In some aspects, the method 1300 may be repeated any number of times for any number of action performances of any number of actions by any number of users to assist any number of patients, as discussed above.

### Example Method for Refining Machine Learning Models to Predict Action Performance Quality for Specific Users

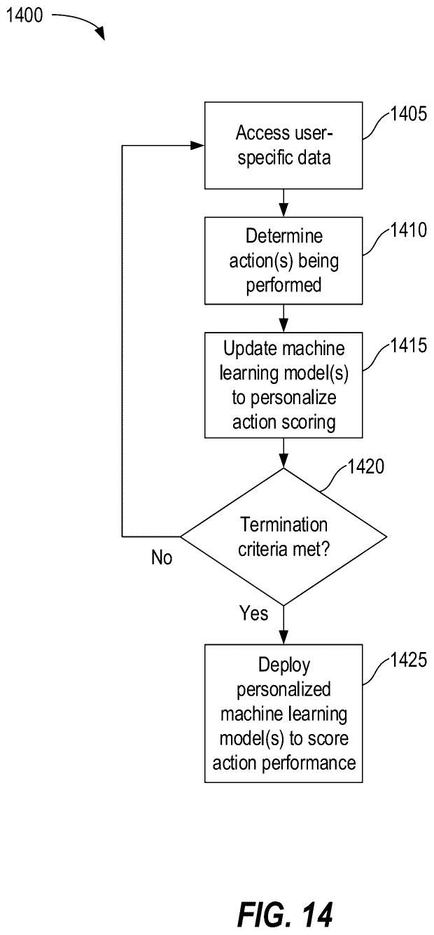

FIG. 14 is a flow diagram depicting an example method 1400 for refining machine learning models to predict action performance quality for specific users, according to some embodiments of the present disclosure. In some aspects, the method 1400 may be performed by a training system, such as the training system 220 of FIGS. 2-3, the training system 520 of FIGS. 5-7, the training system 920 of FIGS. 9-10, and/or the training systems discussed above with reference to FIGS. 12-13. In some aspects, the method 1400 provides additional detail for the workflow 300 of FIG. 3.

At block 1405, the training system accesses user-specific data. For example, as discussed above, the training system may access the sensor data associated with a particular user for whom a personalized model is being trained (e.g., the personalization data 305 of FIG. 3). Generally, the user-specific data is said to be “associated with” the user to indicate that the user-specific data depicts or otherwise reflects the specific user performing action(s). For example, the user-specific data may include images and/or video depicted the user performing the action and/or depicting the patient after the action is performed (e.g., depicting bandaging), audio recording the user performing the action, accelerometer and/or orientation data from the user, and the like. In some embodiments, some or all of the user-specific data may also be associated with other users. For example, if two users simultaneously assist in lifting a patient, the video of the action may be user-specific data for both users. Similarly, in some embodiments, the user-specific data can be associated with the user performing any number and variety of actions for any number of patients.

At block 1410, the training system determines the action(s) being performed, as reflected by one or more exemplars in the user-specific data. For example, as discussed above, the training system may infer the action based on the movements or sensor data itself, or may identify the action based on manual or explicit labeling. For example, in some embodiments, the user may announce or record what actions they are performing (or did perform) to assist a patient. The training system may use this information to determine which action(s) each sample of training data correspond to.

At block 1415, the training system updates one or more machine learning models (e.g., the quality machine learning model 225 of FIG. 3) based on the user-specific data. Generally, updating a machine learning model (also referred to as fine-tuning or refining the model, in some embodiments) includes similar operations to the original training of the model. As discussed above, the particular operations and techniques used to update the machine learning model(s) may vary depending on the particular model architecture and implementation. For example, in the case of neural networks, the training system may process the sensor data of one or more samples of user-specific data as input to the machine learning model(s) to generate an output prediction of performance quality. This prediction can then be compared against a corresponding label(s) of one or more samples in order to generate a loss (e.g., using cross-entropy loss), and the loss can be used to update one or more model parameters (e.g., using backpropagation). This process may be performed iteratively for each user-specific exemplar independently (e.g., using stochastic gradient descent) and/or in batches (e.g., using batch gradient descent).

In some embodiments, the label for some or all of the user-specific data may be determined by the user themselves. For example, when the user performs an action well, they may self-report that the action they just performed is representative of a high quality performance, and should be used to refine their personalized model(s). Similarly, when the user feels they did not perform an action well, they may self-report that the action they just completed was done poorly, and should not be used to train the model (or should be used as a negative exemplar, to personalize the model to quantify what the user considers to be bad performance).

In some embodiments, the label for some or all of the user-specific data may be determined by other users, such as supervising users who observed or assisted with the action performance. For example, as discussed above, the supervising user may record a label indicating the quality of the performance. In some embodiments, the label for some or all of the user-specific data may be determined based on patient input. For example, the patient may report how well they think the action was performed (e.g., whether the user rushed them, moved too quickly or slowly, and the like).

Generally, the user-specific data may be tagged or labeled using any suitable technique or operation to allow the model to be personalized to the specific user. In some embodiments, prior to tagging or using user-specific data to refine the model, the training system may first confirm that the action was not performed poorly. For example, if the (non-personalized) quality model indicates that the action was performed very poorly, it may not be relevant that the user felt it was done well. Similarly, if the patient reports that the action was performed poorly, it may be undesirable to refine the model using the performance as a positive example. In some aspects, therefore, the training system may determine whether the action was performed with at least a minimum level of quality (e.g., based on the prediction generated by the non-personalized model, or based on performance quality reported by individuals other than the user for whom the model is being personalized). If so, the data may be included in the user-specific data to personalize the model. If not, the training system may discard or otherwise refrain from using the data to personalize the model.

At block 1420, the training system determines whether one or more personalization termination criteria have been met. The termination criteria may generally vary depending on the particular implementation, as discussed above. For example, in some embodiments, the training system may determine whether a defined period of time and/or amount of computing resources have been spent personalizing the model(s). In some embodiments, the training system may determine whether a defined number of personalization iterations or epochs have been completed. In some embodiments, the training system may determine whether any additional user-specific samples remain to be used.

If the training system determines that the termination criteria are not met, the method 1400 returns to block 1405 to access more (or new) user-specific data. If, at block 1420, the training system determines that the termination criteria are met, the method 1400 continues to block 1425, where the training system deploys the machine learning model(s) to score action performance during runtime for the specific user. That is, when sensor data for the specific user is received during runtime, the training system may use the personalized model (e.g., the refined quality machine learning model 325 of FIG. 3) to process the sensor data, rather than using the non-personalized model.

In some embodiments, prior to deploying the personalized model, the training system may perform one or more operations to verify its integrity. For example, the training system may process sensor data from the specific user and/or for one or more other users using both the non-personalized model and the personalized model, and verify whether the personalized model generates predictions that are sufficiently similar to the non-personalized model (e.g., whether the average quality scores of the personalized model is within a threshold distance from the scores generated by the non-personalized model). In some embodiments, the training system may confirm that there are no negative exemplars (e.g., sensor data scored as indicating poor action performance) that are scored positively by the personalized model.

If the personalized model fails this verification, in some aspects, the training system may discard it, continue to refine it, or otherwise refrain from deploying the model for inferencing.

### Example Method for Using Machine Learning Models to Predict Action Performance Quality

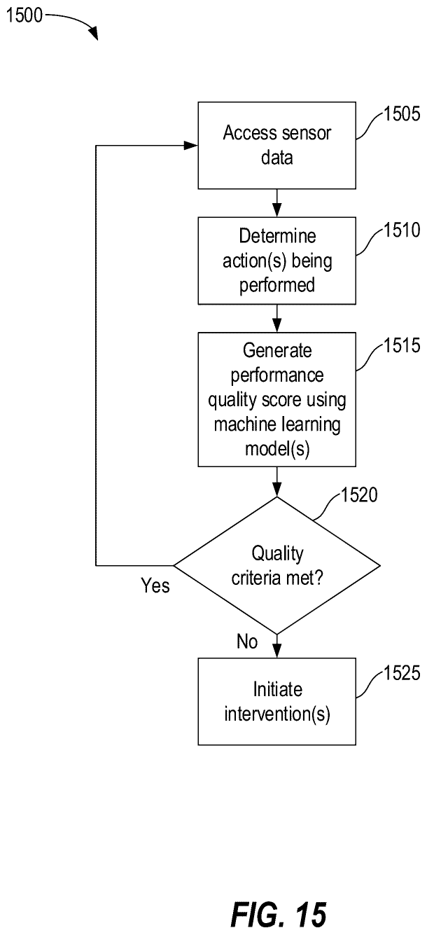

FIG. 15 is a flow diagram depicting an example method 1500 for using machine learning models to predict action performance quality, according to some embodiments of the present disclosure. In some aspects, the method 1500 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and the like. In some aspects, the method 1500 provides additional detail for the workflow 400 of FIG. 4.

At block 1505, the monitoring system accesses sensor data collected while a user performs an action to assist a patient. For example, as discussed above, the monitoring system may access sensor data such as accelerometer data, orientation data, video data, image data, audio data, pressure data, and the like. In some aspects, as discussed above, the monitoring system accesses the sensor data in response to determining that the user is performing (or just completed performance of) the action. Generally, the sensor data (which may correspond to the sensor data 405 of FIG. 4) may comprise any relevant data that may be useful to predict how well the user performed the action.

At block 1510, the monitoring system determines what action(s) were being performed when the sensor data was recorded. For example, as discussed above, the monitoring system may infer the action based on the movements or sensor data itself, or may identify the action based on manual or explicit labeling. For example, in some embodiments, the user may announce or record what actions they are performing (or did perform) to assist a patient. The monitoring system may use this information to determine which action(s) the sensor data corresponds to. In some embodiments, the user may use their personal device before, during and/or after performing the action to indicate its performance (e.g., to indicate the start time, end time, or any other characteristics of the action).

At block 1515, the monitoring system generates a performance quality score (e.g., the quality score 415 of FIG. 4) by processing some or all of the sensor data (accessed at block 1505) using a quality machine learning model (e.g., the quality machine learning model 225 of FIG. 4, and/or a personalized version of the quality machine learning model, as discussed above). In some embodiments, the quality score is generally a numerical value (e.g., between 0 and 1, or between 0 and 100) indicating how well the action was performed (e.g., where higher scores indicate higher quality). In some aspects, as discussed above, performance quality is defined based on how closely the performance reflects the training, instruction, or other representation of a high quality performance (e.g., based on how long one or more parts of the performance took, based on how the user moved during the performance, based on physical pressure exerted on the patient to achieve the action, based on the patient's reported satisfaction, and the like).

At block 1520, the monitoring system determines whether the quality score satisfies one or more defined criteria. For example, the monitoring system may compare the quality score against one or more thresholds to determine whether the quality of the performance was above or below the defined level(s) of quality. In some embodiments, in addition to or instead of evaluating the current performance alone, the monitoring system may evaluate the user's quality scores over time. For example, the monitoring system may determine whether the quality scores are trending upwards or downwards, whether the quality scores have remained above or below one or more thresholds for a threshold period of time (or a threshold number of performances), and the like.

If the criteria are met (e.g., the action was sufficiently high quality), the method 1500 returns to block 1505. If the criteria are not met (e.g., the action was sufficiently poor quality), the method 1500 proceeds to block 1525. At block 1525, the monitoring system initiates one or more interventions for the user based on the poor performance. For example, as discussed above, the monitoring system may notify the user, the patient, a supervisor, and the like.

In this way, the monitoring system can use machine learning to monitor user performance and ensure that patient care remains high quality.

### Example Method for Generating Interventions Based on Machine Learning Evaluations of Action Performance

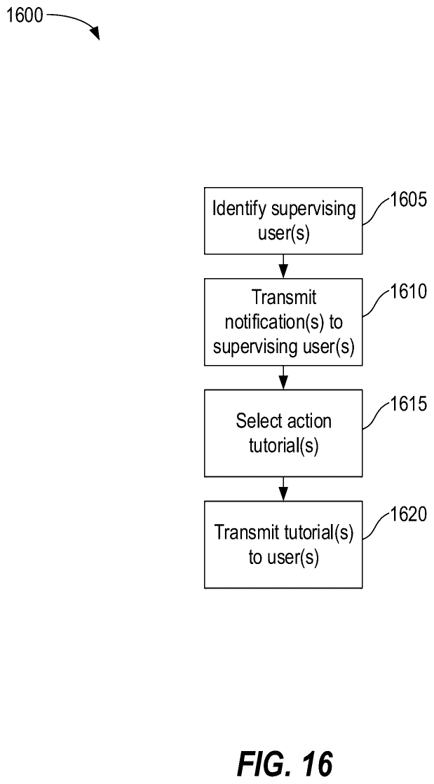

FIG. 16 is a flow diagram depicting an example method 1600 for generating interventions based on machine learning evaluations of action performance, according to some embodiments of the present disclosure. In some aspects, the method 1600 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring system discussed above with reference to FIG. 15. In some aspects, the method 1600 provides additional detail for block 1525 of FIG. 15.

At block 1605, the monitoring system optionally identifies one or more supervising users who are responsible for supervising or otherwise managing the user that performed the action. For example, the monitoring system may evaluate one or more mappings or other data to identify users to whom the acting user reports.

At block 1610, the monitoring system optionally transmits one or more notifications to the supervising user(s). For example, as discussed above, the notification may indicate what action was being performed, what user performed it, what patient was being assisted, when the action was performed, where the action was performed, and the like. In some embodiments, the notification may include further information such as some or all of the sensor data (e.g., imagery or video), the generated performance quality score, and the like. This may allow a supervising user to check in with the acting user in order to ensure they are performing well.

At block 1615, the monitoring system optionally selects one or more action tutorials or instructions for the action(s) that was being performed. For example, the monitoring system may select an instructional video discussing how to best perform the action, or may select a diagram or pamphlet depicting how to perform the action. In some embodiments, the monitoring system selects the action tutorial based on factors such as what information the user has already reviewed (e.g., to select a new piece of material), what modalities or types of media the user prefers to engage with, and the like.

At block 1620, the monitoring system optionally transmits the selected tutorial(s) (or a link or pointer to the tutorial) to the user that performed the action. For example, the monitoring system may provide a link to watch the video, or provide a calendar invite for in-person training with respect to the action.

Generally, each intervention discussed in the present disclosure may be an optional response to action performance. That is, the actions discussed herein are intended to be used as examples, and are not limiting on aspects of the present disclosure. In embodiments, the monitoring system may initiate more or fewer interventions, and may generally take any appropriate action in response to the user's performance.

### Example Method for Training Machine Learning Models to Predict Patient Outcomes Based on Sensor Data

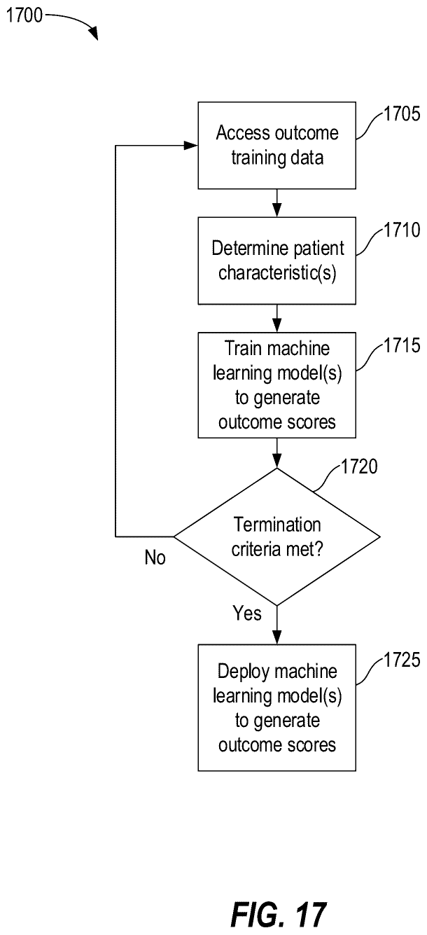

FIG. 17 is a flow diagram depicting an example method 1700 for training machine learning models to predict patient outcomes based on sensor data, according to some embodiments of the present disclosure. In some aspects, the method 1700 may be performed by a training system, such as the training system 220 of FIGS. 2-3, the training system 520 of FIGS. 5-7, the training system 920 of FIGS. 9-10, and/or the training systems discussed above with reference to FIGS. 12-14. In some aspects, the method 1700 provides additional detail for the workflow 500 of FIG. 5.

At block 1705, the training system accesses outcome training data. Generally, the outcome training data comprises information related to outcome(s) experienced by one or more patients as a result of actions performed by one or more users to assist the one or more patients. For example, the outcome training data may correspond to the training data 505 of FIG. 5. In some embodiments, as discussed above, the outcome training data may include a set of training samples or exemplars, where each sample includes sensor data depicting or indicative of the action performance (e.g., the sensor data 510 of FIG. 5), as well as a label indicating the outcome(s) experienced by the patient(s) (e.g., the outcome labels 515 of FIG. 5).

As discussed above, the sensor data may generally include any relevant data, collected via any suitable sensor or device, which may be useful in identifying action(s) being performed and/or in determining how the action was performed. For example, the sensor data may include data from one or more wearable sensors on the user and/or patient (e.g., acceleration information, orientation information, pressure information, and the like), data from one or more sensors in the physical space or room of the patient (e.g., pressure sensors, cameras, microphones, and the like), and any other relevant data.

The outcome labels are generally indicative of what outcomes the patient experienced. As discussed above, in some embodiments, the outcome labels correspond to outcomes that can reasonably be said to have been caused by the action performance. That is, in some embodiments, the outcome labels may include outcomes for which the proximate cause was the way in which the action was performed, but may exclude outcomes which may have occurred regardless of how (or whether) the action was performed. For example, it may be reasonably stated that a patient should not ordinarily suffer bruising as a result of being lifted by a user. Therefore, if the patient suffers bruising in the area where the user lifted them, it may be reasonably inferred that the way in which the user performed the lifting caused the bruising. As another example, it may be reasonably stated that a pressure sore may result from a user leaving a patient on one side for an extended period of time. However, if the sensor data indicates that the user left the patient on the opposite side from where the sore developed, it cannot reasonably be stated that the user's action caused the pressure sore. As another example, the comfort of the patient shortly after the action is performed is likely to be directly related to how the action was performed.

In some embodiments, therefore the outcome labels may be generated at least in part based on a set of rules or mappings indicating which outcome(s) are the potential result of any given action. In some embodiments, the outcomes experienced by a patient may be further evaluated to determine whether they are, in fact, linked to the action performance. For example, if the sensor data indicates that the user used extremely little pressure or force, the training system (or another system) may determine that bruising of the patient was not caused by the user. In some embodiments, some or all of these mappings and outcomes may be evaluated automatically without explicit user input. In some embodiments, some or all of these mappings may be evaluated using manual input. For example, the patient and/or user may indicate that the performance of the action caused the outcome, or a supervising user or other individual may make this determination.

At block 1710, the training system determines a set of patient characteristics corresponding to one or more exemplars in the training data. For example, as discussed above, some patient characteristics such as age, comorbidities, mobility, weight, and the like may have some relevance as to what outcome(s) may be caused by performed action(s). In some embodiments, by using such characteristics as supplemental input to the machine learning model (along with the sensor data), the model may learn to generate more accurate assessments. For example, the model may learn that a certain outcome (e.g., a fractured bone) is more likely to occur when certain comorbidities (e.g., osteoporosis) are present in the patient. Therefore, the model may learn that use of a given technique (e.g., a given amount of pressure used to help the patient stand) may cause negative outcomes to patients having such comorbidities, even if it would be non-injurious to other patients lacking these comorbidities.

At block 1715, the training system trains one or more machine learning models (e.g., the outcome machine learning model 525 of FIGS. 2 and/or 3) based on the training data. Generally, as discussed above, the particular operations and techniques used to train the machine learning model(s) may vary depending on the particular model architecture and implementation. For example, in the case of neural networks, the training system may process the sensor data of one or more samples of training data as input to the machine learning model(s) to generate an output prediction of outcome(s) the patient may experience as a result of the action performance.

This prediction can then be compared against the corresponding label(s) of one or more samples in order to generate a loss (e.g., using cross-entropy loss), and the loss can be used to update one or more model parameters (e.g., using backpropagation). This process may be performed iteratively for each training exemplar independently (e.g., using stochastic gradient descent) and/or in batches (e.g., using batch gradient descent).

In this way, the model can iteratively learn to predict the potential outcome risks of action performance based on the sensor data collected while the user performs the action.

At block 1720, the training system determines whether one or more training termination criteria have been met. The termination criteria may generally vary depending on the particular implementation. For example, in some embodiments, the training system may determine whether a defined period of time and/or amount of computing resources have been spent training the model(s). In some embodiments, the training system may determine whether the model(s) have reached a defined or preferred minimum accuracy. In some embodiments, the training system may determine whether a defined number of training iterations or epochs have been completed. In some embodiments, the training system may determine whether any additional training samples remain to be used.

If the training system determines that the termination criteria are not met, the method 1270 returns to block 1705 to access more (or new) training data. If, at block 1720, the training system determines that the termination criteria are met, the method 1700 continues to block 1725, where the training system deploys the machine learning model(s) to quantify outcome risk(s) during runtime. As discussed above, deploying the model(s) may generally include any operations used to prepare or provide the model to process input data (e.g., new sensor data) during runtime. For example, the training system may instantiate the model locally, or may transmit the model details (e.g., the values of the learned parameters) to one or more other systems for inferencing.

### Example Method for Generating Training Data for Machine Learning Models to Predict Patient Outcomes Based on Sensor Data

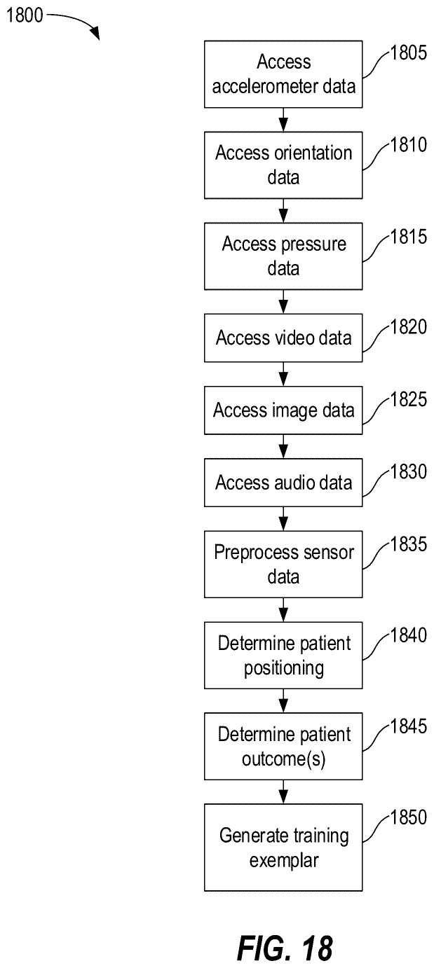

FIG. 18 is a flow diagram depicting an example method 1800 for generating training data for machine learning models to predict patient outcomes based on sensor data, according to some embodiments of the present disclosure. In some aspects, the method 1800 may be performed by a training system, such as the training system 220 of FIGS. 2-3, the training system 520 of FIGS. 5-7, the training system 920 of FIGS. 9-10, and/or the training systems discussed above with reference to FIGS. 12-14 and/or 17. In some aspects, the method 1800 provides additional detail for block 1705 of FIG. 17.

At block 1805, the training system accesses accelerometer data collected via one or more sensors as a user performs a task for a patient. For example, as discussed above, the accelerometer data may be collected via accelerometers embedded in one or more wearable devices, such as on the user's wrist(s), on the patient, and the like. In some aspects, the accelerometer data is collected from other devices or components, such as a smartphone of the user and/or patient, or one or more tools or devices used to provide the assistance (e.g., an accelerometer in a walker or cane used to help the patient walk).

At block 1810, the training system accesses orientation data collected via one or more orientation sensors as the user performs the task. In some embodiments, the orientation data may be collected from similar (or the same) devices to the accelerometer data. For example, the orientation data may be collected via orientation sensors embedded in one or more wearable devices, such as on the user's wrist(s), on the patient, and the like. In some aspects, the orientation data is collected from other devices or components, such as a smartphone of the user and/or patient, or one or more tools or devices used to provide the assistance (e.g., an orientation sensor in a walker or cane used to help the patient walk).

At block 1815, the training system accesses pressure data collected via one or more sensors during or after the user performs the task. For example, as discussed above the pressure data may be collected via pressure sensors to indicate information such as the grip strength of the user, the positioning of the patient, the amount of pressure exerted on the patient by the user, and the like.

At block 1820, the training system accesses video data depicting the user performing the action for the patient. For example, as discussed above, one or more video cameras may capture the performance from one or more angles in the space. In some embodiments, the video can include multiple types of video, such as color video for visible light (e.g., red, green, and blue (RGB)) video, video with depth information (e.g., RGB-Depth (RBG-D)) video, stereoscopic video, thermal video, and the like. In some embodiments, the user and/or patient may be prompted to capture the video data during or after performance of the action (e.g., to confirm that it was completed).

At block 1825, the training system accesses image data depicting the user performing the action for the patient. In some embodiments, the image data may be collected if video data is not available (e.g., due to insufficient bandwidth to transmit full video data, or because the room does not contain a video camera). For example, a video may be captured, and a subset of the frames from the video may be accessed as training data. For example, as discussed above, one or more video cameras may capture the performance from one or more angles in the space. In some embodiments, the image data can include multiple types of images, such as color images in the visible light spectrum, images with depth information, stereoscopic images, thermal images, and the like. In some embodiments, the user and/or patient may be prompted to capture the image data during or after performance of the action (e.g., to confirm that it was completed).

At block 1830, the training system accesses audio data of the user performing the action for the patient. For example, as discussed above, one or more microphones may capture the performance from one or more positions in the space. In some embodiments, the audio data includes audio in the audible frequency range. Generally, the audio data may include natural language speech (e.g., of the user and/or patient) as well as non-language audio (e.g., sounds caused by the action performance, such as a shower running, a pot boiling, a vacuum running, a toilet flushing, and the like). In some embodiments, the user and/or patient may be prompted to capture the audio data during or after performance of the action (e.g., to confirm that it was completed).

Although a number of data modalities (e.g., including accelerometer, orientation, pressure, video, image, and audio data) are depicted for conceptual clarity, these modalities are intended to be used as examples, and are not limiting on embodiments of the present disclosure. That is, the training system may use more or fewer modalities than those contemplated herein, and may generally use any relevant or useful sensor data.

At block 1835, the training system optionally preprocesses the sensor data if appropriate to render the data more suited for processing using machine learning models. Generally, the particular operations used for preprocessing may vary depending on the particular implementation and modality of the data. For example, the training system may remove outlier data, denoise the data, delineate the data into windows or points in time, aggregate the data, and the like.

At block 1840, the training system determines patient positioning as a result of the action performance. For example, the training system may evaluate the sensor data (e.g., image or video data, pressure data, orientation data, and the like) and/or user or patient input to determine which position(s) the patient was in before, during, and/or after the action was performed. In some embodiments, as discussed above, this positioning data may be used as target output for one machine learning model (e.g., the position machine learning model 635 of FIG. 6) based on sensor data input.

At block 1845, the training system determines any outcome(s) experienced by the patient. In some embodiments, as discussed above, the training system may use a set of mappings between outcomes and actions to determine which outcome(s) may have been caused by the performed action(s). In some embodiments, the training system may further evaluate mappings or other information about what aspects of action performance may cause the outcome. For example, a mapping may indicate that pressure sores are only (potentially) caused by the action if the user left the patient on the side where the sores developed. In some aspects, as discussed above, this outcome data may be used as target output for a second machine learning model (e.g., the result machine learning model 640 of FIG. 6) based on patient positioning input.

Although not depicted in the illustrated example, in some aspects, the training system may similarly determine any relevant patient characteristics, as discussed above. These characteristics may be used as input to an outcome machine learning model, a positioning machine learning model, and/or a result machine learning model, as discussed above.

At block 1850, the training system generates a training exemplar including the accessed sensor data (e.g., from each modality used) as the input portion of the exemplar, as well as the determined positioning and/or outcomes as the target output or label of the exemplar. In some embodiments, the training system may alternatively generate a training exemplar including the sensor data and positioning information as input, and the patient outcome(s) as target output. In some embodiments, the training system may alternatively generate a first training exemplar using the sensor data as input and the positioning as target output, and further generate a second training exemplar using the positioning as input and the resulting outcome(s) as target output.

In this way, the training exemplar(s) may be stored and/or used to train or refine one or more outcome machine learning models, as discussed above. In some aspects, the method 1800 may be repeated any number of times for any number of action performances of any number of actions by any number of users to assist any number of patients, as discussed above.

### Example Method for Training a Set of Machine Learning Models to Predict Patient Position and Patient Outcomes

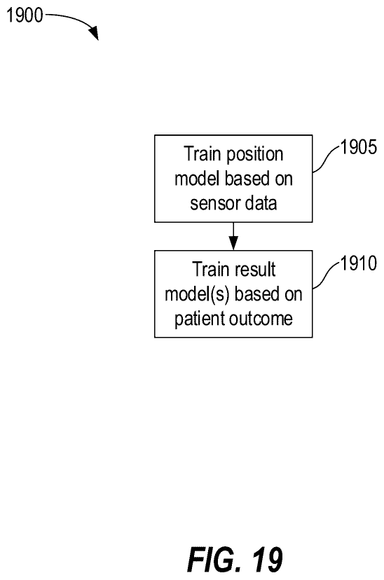

FIG. 19 is a flow diagram depicting an example method 1900 for training a set of machine learning models to predict patient position and patient outcomes, according to some embodiments of the present disclosure. In some aspects, the method 1900 may be performed by a training system, such as the training system 220 of FIGS. 2-3, the training system 520 of FIGS. 5-7, the training system 920 of FIGS. 9-10, and/or the training systems discussed above with reference to FIGS. 12-14 and/or 17-18. In some aspects, the method 1900 provides additional detail for the workflow 600 of FIG. 6 and/or for block 1715 of FIG. 17.

At block 1905, the training system trains a position model (e.g., the position machine learning model 635 of FIG. 6) based on sensor data. For example, as discussed above, the training system may process one or more aspects of the sensor data (e.g., orientation data, pressure data, and the like) as input to the model in order to generate an output prediction as to what position(s) the patient was in before, during, and/or after performance of the corresponding action. This prediction can then be compared against the actual position data, indicating what position(s) the patient was actually in, to generate a loss. The loss can then be used to refine the position machine learning model, such that the position machine learning model learns to predict patient positioning based on sensor data corresponding to action performance.

At block 1910, the training system trains a result model (e.g., the result machine learning model 640 of FIG. 6) based on patient outcomes. For example, as discussed above, the training system may process the patient position information (e.g., the ground truth information indicating the actual patient position(s)) as input to the model in order to generate an output prediction as to what outcome(s) the patient may suffer. These predicted outcome(s) can then be compared against the actual outcome data to generate a loss, and the loss can be used to refine the result machine learning model. In this way, the result machine learning model learns to predict what outcome(s) may occur as a result of patient positioning before, during, and/or after action performance.

As discussed above, these models may then be jointly used as an ensemble outcome model to predict patient outcomes based on user actions, as discussed above. For example, the position model may predict that if the user performs a given action in a specific way, the patient is likely to have their left arm twisted in a particular way. This prediction may then be processed using the result model to predict what outcome(s) may be caused as a result of this arm twist. As another example, the position model may predict that if the user performs the action one way, the patient will be left on their left side. This position information (e.g., indicating the patient positioning before and/or after the action) may be evaluated using the result model to predict whether the patient will suffer from pressure sores or any other negative outcome, as discussed above.

### Example Method for Training a Set of Machine Learning Models to Predict Relevant Patient Outcomes

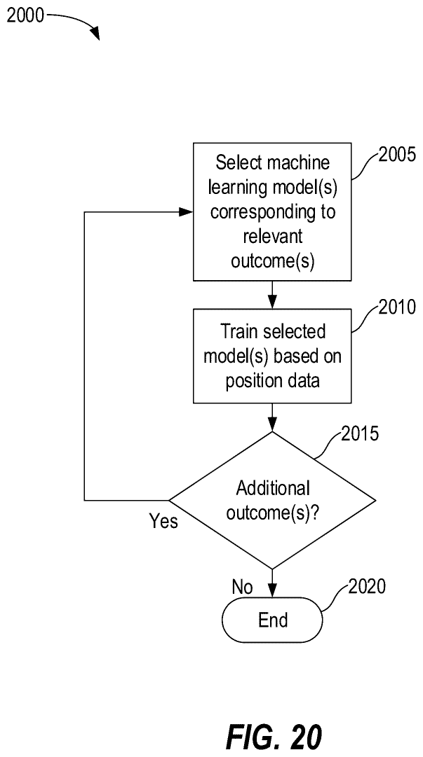

FIG. 20 is a flow diagram depicting an example method 2000 for training a set of machine learning models to predict relevant patient outcomes, according to some embodiments of the present disclosure. In some aspects, the method 2000 may be performed by a training system, such as the training system 220 of FIGS. 2-3, the training system 520 of FIGS. 5-7, the training system 920 of FIGS. 9-10, and/or the training systems discussed above with reference to FIGS. 12-14 and/or 17-19. In some aspects, the method 2000 provides additional detail for block 1715 of FIG. 17 and/or block 1910 of FIG. 19.

At block 2005, the training system selects one or more machine learning models that correspond to the relevant outcome(s) experienced by a patient. That is, if the training system is training model(s) based on patient outcomes, the training system may determine which outcome(s) the patient actually experienced (e.g., indicated in the selected or current training exemplar), and may then select the corresponding model(s) that are being trained to predict this specific outcome. In some embodiments, this may allow the training system to train outcome-specific models (e.g., where each outcome is predicted using a specific model trained based on data for that outcome). This may result in substantially improved prediction accuracy, as each model may specialize and learn the specific correlations with respect to the specific outcome.

In some embodiments, as discussed above, during inferencing the system may therefore process sensor data and/or positioning data using a set of models (e.g., one for each potential outcome) to generate a set of predictions. For example, the inferencing system may process the sensor data using a first model to predict patient positioning, and may determine a set of relevant outcome models (e.g., models that predict outcomes which are relevant to or may be caused by the specific action that was performed, such as determined based on rules or mappings). By processing the positioning data using this set of models, the inferencing system can predict specific relevant outcomes in a more accurate and robust way.

At block 2010, the training system trains the selected model(s) based on the position data indicated in the exemplar. That is, as discussed above, the training system trains one or more outcome-specific models corresponding to the actual outcome(s) that occurred, using the position data (e.g., indicating what position(s) the patient was in before, during, and/or after the action performance) as input and the actual outcome(s) as target output.

At block 2015, the training system determines whether there are one or more additional outcome(s), experienced by the patient, which have not been used to train a corresponding model. If so, the method 2000 returns to block 2005 to select the model(s) corresponding to the one or more other outcome(s). If not, the method 2000 terminates at block 2020. As discussed above, these outcome-specific models may be substantially more accurate, in some embodiments.

### Example Method for Refining Machine Learning Models to Predict Patient Outcomes for Specific Patients

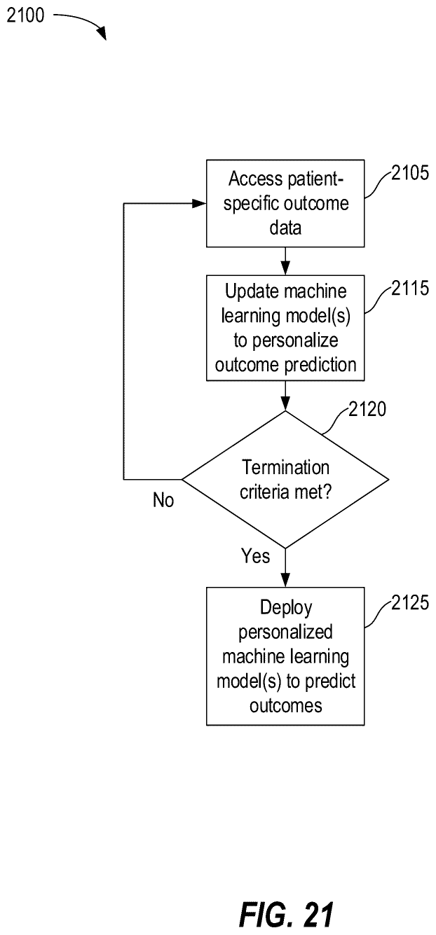

FIG. 21 is a flow diagram depicting an example method 2100 for refining machine learning models to predict patient outcomes for specific patients, according to some embodiments of the present disclosure. In some aspects, the method 2100 may be performed by a training system, such as the training system 220 of FIGS. 2-3, the training system 520 of FIGS. 5-7, the training system 920 of FIGS. 9-10, and/or the training systems discussed above with reference to FIGS. 12-14 and/or 17-20. In some aspects, the method 2100 provides additional detail for the workflow 700 of FIG. 7.

At block 2105, the training system accesses patient-specific outcome data. For example, as discussed above, the training system may access the outcome data associated with a particular patient for whom a personalized model is being trained (e.g., the personalization data 705 of FIG. 7). Generally, the patient-specific data is said to be “associated with” the patient to indicate that the patient-specific data reflects the specific outcome(s) experienced by the specific patient. For example, the patient-specific data may include medical notes discussing or diagnosing the outcomes of the patient.

At block 2115, the training system updates one or more machine learning models (e.g., the outcome machine learning model 525 of FIG. 5) based on the patient-specific data. Generally, updating a machine learning model (also referred to as fine-tuning or refining the model, in some embodiments) includes similar operations to the original training of the model, as discussed above. The particular operations and techniques used to update the machine learning model(s) may vary depending on the particular model architecture and implementation. For example, in the case of neural networks, the training system may process the positioning data of one or more samples of patient-specific data as input to the machine learning model(s) to generate an output prediction of potential outcome(s). This prediction can then be compared against the corresponding label(s) of one or more samples in order to generate a loss (e.g., using cross-entropy loss), and the loss can be used to update one or more model parameters (e.g., using backpropagation). This process may be performed iteratively for each patient-specific exemplar independently (e.g., using stochastic gradient descent) and/or in batches (e.g., using batch gradient descent).

At block 2120, the training system determines whether one or more personalization termination criteria have been met. The termination criteria may generally vary depending on the particular implementation, as discussed above. For example, in some embodiments, the training system may determine whether a defined period of time and/or amount of computing resources have been spent personalizing the model(s). In some embodiments, the training system may determine whether a defined number of personalization iterations or epochs have been completed. In some embodiments, the training system may determine whether any additional patient-specific samples remain to be used.

If the training system determines that the termination criteria are not met, the method 2100 returns to block 2105 to access more (or new) patient-specific data. If, at block 2120, the training system determines that the termination criteria are met, the method 2100 continues to block 2125, where the training system deploys the machine learning model(s) to predict outcomes during runtime for the specific patient. That is, when sensor data indicating action performance and/or position data for the specific patient is received during runtime, the inferencing system may use the personalized model (e.g., the refined outcome machine learning model 725 of FIG. 7) to process the sensor data and/or position data, rather than using the non-personalized model.

In some embodiments, as discussed above, the system may use one or more patient characteristics as input to the model. In some embodiments, by personalizing the model specifically to the particular patient, the system may obviate the need to use such input data, as the model may learn to account for such characteristics inherently (in the process of being personalized).

### Example Method for Using Machine Learning Models to Predict Patient Outcomes Based on Sensor Data

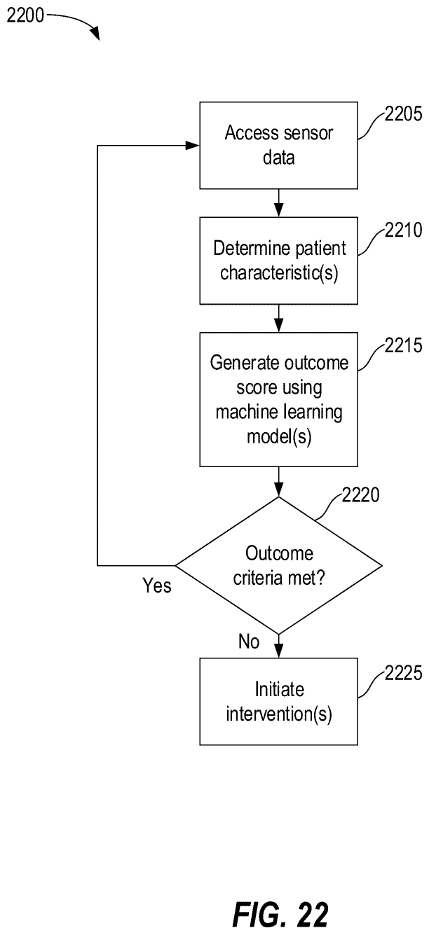

FIG. 22 is a flow diagram depicting an example method 2200 for using machine learning models to predict patient outcomes based on sensor data, according to some embodiments of the present disclosure. In some aspects, the method 2200 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16. In some aspects, the method 2200 provides additional detail for the workflow 800 of FIG. 8.

At block 2205, the monitoring system accesses sensor data collected while a user performs an action to assist a patient. For example, as discussed above, the monitoring system may access sensor data such as accelerometer data, orientation data, video data, image data, audio data, pressure data, and the like. In some aspects, as discussed above, the monitoring system accesses the sensor data in response to determining that the user is performing (or just completed performance of) the action. Generally, the sensor data (which may correspond to the sensor data 805 of FIG. 8) may comprise any relevant data that may be useful to predict or determine how the user performed the action (e.g., what position(s) the performance may put the patient in, what outcome(s) the patient may experience, and the like).

At block 2210, the monitoring system determines a set of patient characteristics for the patient for whom the action was performed, as of the time when the sensor data was recorded. For example, as discussed above, the monitoring system may access patient data (e.g., the patient data 807 of FIG. 8) to determine the relevant characteristics of the patient when the action was performed (e.g., excluding characteristics such as disorders or comorbidities that developed subsequent to the action performance). As discussed above, the particular relevant characteristics may vary depending on the particular implementation. For example, some characteristics may be relevant for some actions and irrelevant for others. Similarly, some characteristics may be relevant for some outcomes and irrelevant for others.

At block 2115, the monitoring system generates one or more outcome scores (e.g., the outcome score 815 of FIG. 8) by processing some or all of the sensor data (accessed at block 2205) using an outcome machine learning model (e.g., the outcome machine learning model 525 of FIG. 5, and/or a personalized version of the outcome machine learning model, as discussed above). In some embodiments, the outcome score generally comprises a set of one or more numerical values (e.g., between 0 and 1, or between 0 and 100) indicating how likely one or more outcomes are to occur (e.g., where higher scores indicate higher probability). In some aspects, as discussed above, the outcome prediction(s) are generated using a set of outcome models, one for each outcome, to predict the probability of each.

At block 2220, the monitoring system determines whether the outcome score(s) satisfy one or more defined criteria. For example, the monitoring system may compare the outcome score against one or more thresholds to determine whether any outcome(s) are substantially likely (e.g., with a score that is above a defined threshold). In some aspects, the monitoring system compares the outcome scores to determine whether the outcome(s) are sufficiently unlikely. That is, evaluating the criteria may include determining whether the score exceeds a threshold (e.g., indicating that an outcome is sufficiently likely) and/or whether the score is below a threshold (e.g., indicating that the outcome is sufficiently unlikely).

If the criteria are met (e.g., the outcome(s) are sufficiently unlikely), the method 2200 returns to block 2205. If the criteria are not met (e.g., one or more outcomes are sufficiently likely), the method 2200 proceeds to block 2225. At block 2225, the monitoring system initiates one or more interventions for the patient and/or user based on the potential outcomes. For example, as discussed above, the monitoring system may notify the user, the patient, a supervisor, and the like.

In this way, the monitoring system can use machine learning to monitor user performance and ensure that patient care remains high quality.

### Example Method for Using a Set of Machine Learning Models to Predict Patient Position and Patient Outcomes

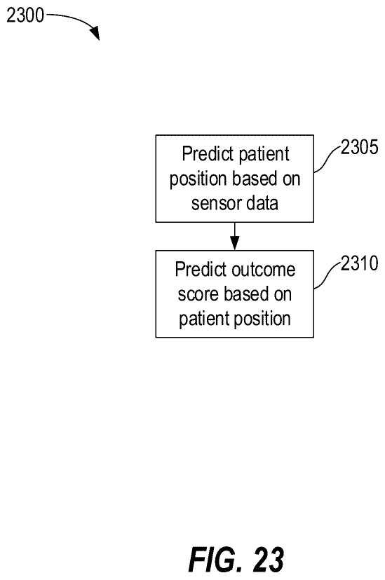

FIG. 23 is a flow diagram depicting an example method 2300 for using a set of machine learning models to predict patient position and patient outcomes, according to some embodiments of the present disclosure. In some aspects, the method 2300 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16 and/or 22. In some aspects, the method 2300 provides additional detail for block 2215 of FIG. 22.

At block 2305, the monitoring system predicts one or more patient position(s) based on the sensor data (e.g., accessed at block 2205 of FIG. 22). For example, as discussed above, the monitoring system may process some or all of the sensor data using one or more position models (e.g., the position machine learning model 635 of FIG. 6) to determine or predict what position(s) the patient was, is, or will be in before, during, and/or after the performance of the action, as reflected by the sensor data. As discussed above, the predicted position can generally include any level of granularity depending on the particular implementation. For example, the monitoring system may predict whether the patient is likely to be left standing, seated upright, reclined (and/or the extent of recline), supine, and the like.

In some embodiments, the predicted position may include the predicted orientation of the patient before, during, and/or after the performance. For example, the monitoring system may predict whether the user will be on their left side, their right side, their back, their stomach, and the like. In some embodiments, the predicted position may include predicting positions and/or orientations of one or more limbs of the patient, such as to predict how high their leg will be lifted, how far back their arm is pulled, and the like.

At block 2310, the monitoring system predicts one or more outcome scores for the patient based on the predicted position(s). For example, as discussed above, the monitoring system may process some or all of the predicted position(s) using a result model (e.g., the result machine learning model 640 of FIG. 6) to predict the probability of one or more outcomes occurring based on the positioning. In some embodiments, as discussed above, predicting the outcome score(s) includes processing the position data with one or more outcome-specific models. In some embodiments, as discussed above, predicting the outcome(s) includes processing the position data with one or more patient-specific models.

Generally, using the method 1900, the monitoring system can thereby predict what outcome(s) may occur to the patient based on the specific patient, as well as based on how the action was performed, which may substantially improve patient outcomes (e.g., allowing the user to immediately remedy any concerns as they occur, rather than waiting for the eventual outcomes to occur).

### Example Method for Using a Set of Machine Learning Models to Predict Relevant Patient Outcomes

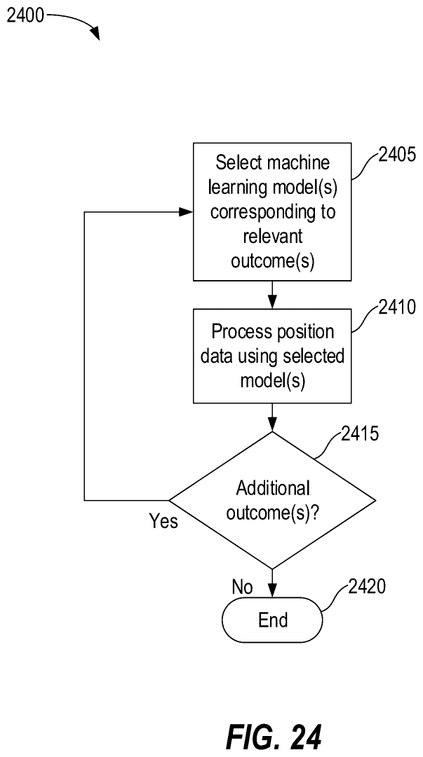

FIG. 24 is a flow diagram depicting an example method 2400 for using a set of machine learning models to predict relevant patient outcomes, according to some embodiments of the present disclosure. In some aspects, the method 2400 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16 and/or 22-23. In some aspects, the method 2400 provides additional detail for block 2215 of FIG. 22 and/or block 2310 of FIG. 23.

At block 2405, the monitoring system selects one or more machine learning models that correspond to the relevant outcome(s) for which the monitoring system is generating a prediction. That is, the monitoring system may determine which outcome(s) the patient will potentially experience as a result of the action performance, and may then select the corresponding model(s) that are trained to predict these specific outcomes. For example, as discussed above, the monitoring system may evaluate one or more mappings indicating what outcome(s) may be caused by various action(s). Based on the action(s) that were performed (or are being performed) for the patient, the monitoring system may identify the relevant outcome model(s).

In some embodiments, as discussed above, the monitoring system may process the sensor data using a first model to predict patient positioning, and may process this predicted positioning data using the selected set of model(s), to predict specific relevant outcomes in a more accurate and robust way.

At block 2410, the monitoring system processes the position data using the selected model(s) to generate a set of one or more outcome score(s) indicating the predicted probability that each outcome will occur, based on the action performance.

At block 2415, the monitoring system determines whether there are one or more additional outcome(s), which may be experienced by the patient, which have not been evaluated. If so, the method 2400 returns to block 2405 to select the model(s) corresponding to the one or more other outcome(s). If not, the method 2400 terminates at block 2420. As discussed above, these outcome-specific models may be substantially more accurate, in some embodiments.

### Example Method for Generating Interventions Based on Machine Learning Evaluations of Patient Outcomes

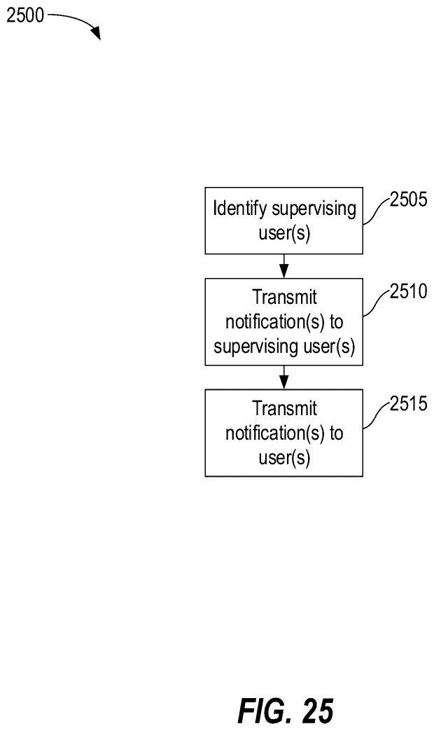

FIG. 25 is a flow diagram depicting an example method 2500 for generating interventions based on machine learning evaluations of patient outcomes, according to some embodiments of the present disclosure. In some aspects, the method 2500 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16 and/or 22-24. In some aspects, the method 2500 provides additional detail for block 2225 of FIG. 22.

At block 2505, the monitoring system optionally identifies one or more supervising users who are responsible for supervising or otherwise managing the user that performed (or is performing) the action. For example, the monitoring system may evaluate one or more mappings or other data to identify users to whom the acting user reports.

At block 2510, the monitoring system optionally transmits one or more notifications to the supervising user(s). For example, as discussed above, the notification may indicate what action was (or is) being performed, what user performed (or is performing) it, what patient was (or is) being assisted, when the action was performed, where the action was performed, and the like. In some embodiments, the notification may include further information such as some or all of the sensor data (e.g., imagery or video), the generated outcome score(s), and the like. This may allow a supervising user to check in with the acting user in order to ensure the patient is treated well.

At block 2515, the monitoring system transmits one or more notifications to the user that performed (or is performing) the action. For example, the monitoring system may notify the user that the action (or the way the action was performed, such as the position(s) the patient was placed in) may cause the predicted outcome(s) to occur. In some embodiments, these notifications are provided in real-time (or near real-time), such as while the user is still performing the action. For example, the user's smartphone or wearable may vibrate, ring, or otherwise output a notification or alert to the user, allowing them to immediately review the information to determine how to proceed.

For example, the notification may inform the user that the patient was already on their left side, and appears to remain on their left side after the action (or is predicted to end on their left side), and that pressure sores may result. Therefore, the user may determine to reposition the patient immediately to prevent this harm. As another example, the notification may inform the user that this particular patient has one or more joint issues that cause pain if their shoulder is rotated beyond a specific amount, and it appears that the user is about to rotate the patient's shoulder beyond this limit. In response, the user may stop this motion to prevent this harm to the patient.

Generally, each intervention discussed in the present disclosure may be an optional response to the potential outcomes. That is, the actions discussed herein are intended to be used as examples, and are not limiting on aspects of the present disclosure. In embodiments, the monitoring system may initiate more or fewer interventions, and may generally take any appropriate action in response to the potential outcomes.

### Example Method for Training Machine Learning Models to Predict Action Injury Risk

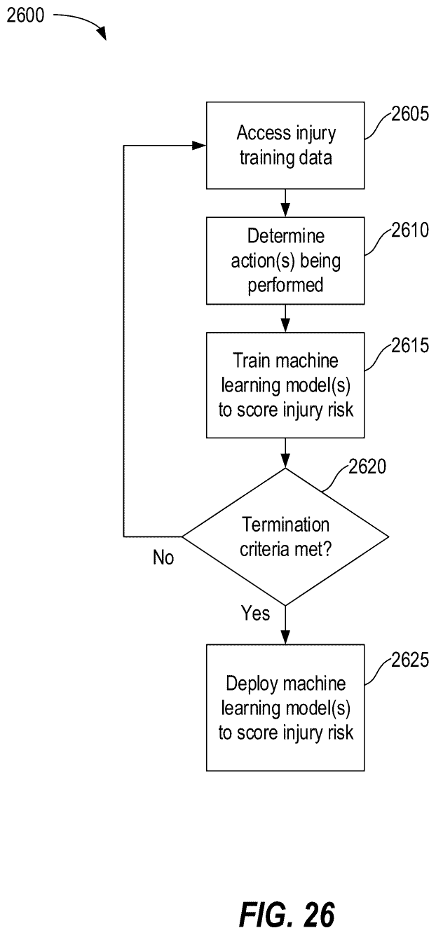

FIG. 26 is a flow diagram depicting an example method 2600 for training machine learning models to predict action injury risk, according to some embodiments of the present disclosure. In some aspects, the method 2600 may be performed by a training system, such as the training system 220 of FIGS. 2-3, the training system 520 of FIGS. 5-7, the training system 920 of FIGS. 9-10, and/or the training systems discussed above with reference to FIGS. 12-14 and/or 17-21. In some aspects, the method 2600 provides additional detail for the workflow 900 of FIG. 9.

At block 2605, the training system accesses injury training data. Generally, the injury training data comprises information related to actions performed by one or more users to assist one or more patients, as well as one or more injuries (or potential injuries) of the user that resulted from the action performance. For example, the injury training data may correspond to the training data 905 of FIG. 9. In some embodiments, as discussed above, the injury training data may include a set of training samples or exemplars, where each sample includes sensor data depicting or indicative of the action performance (e.g., the sensor data 910 of FIG. 9), as well as a label indicating the associated risk(s) of injury (e.g., the injury labels 915 of FIG. 9).

As discussed above, the sensor data may generally include any relevant data, collected via any suitable sensor or device, which may be useful in identifying action(s) being performed and/or in quantifying how risk the action performance is (with respect to injury to the user performing the action). For example, the sensor data may include data from one or more wearable sensors on the user and/or patient (e.g., acceleration information, orientation information, pressure information, and the like), data from one or more sensors in the physical space or room of the patient (e.g., pressure sensors, cameras, microphones, and the like), and any other relevant data.

The injury labels are generally indicative of what injuries were caused by the action performance and/or what injuries the user was at risk of. For example, the injury labels may indicate whether the user suffered any condition or disorder such as a sprained wrist, injured back, and the like. In some aspects, as discussed above, the injury labels only indicate injuries that were caused by (or are believed to have been caused by) the action performance itself. For example, a strained back may be caused by lifting the patient. Accordingly, if the action involved helping the patient move or transfer between chairs and/or beds, and the user reports having a strained back, it may be inferred that the way the action was performed (e.g., how the patient was lifted) caused the injury (a strained back).

As discussed above, in some embodiments, the injury labels are determined based on defined mappings. For example, some injuries may be defined as only occurring (or reasonably occurring) when one or more particular actions (e.g., transferring the patient) are performed incorrectly. In some embodiments, therefore, when such injuries are noted, the training system (or another system) may generate a training exemplar that labels the corresponding sensor data (collected when the action or actions were performed) with the noted injury (or injuries). In this way, the system can automatically build a set of training data based on defined action-injury mappings.

At block 2610, the training system determines which action(s) were being performed for one or more of the exemplars of training data. For example, as discussed above, the training system may infer the action based on the movements or sensor data itself, or may identify the action based on manual or explicit labeling. For example, in some embodiments, the user may announce or record what actions they are performing (or did perform) to assist a patient. The training system may use this information to determine which action(s) each sample of training data correspond to.

At block 2615, the training system trains one or more machine learning models (e.g., the injury machine learning model 925 of FIG. 9) based on the training data. Generally, as discussed above, the particular operations and techniques used to train the machine learning model(s) may vary depending on the particular model architecture and implementation. For example, in the case of neural networks, the training system may process the sensor data of one or more samples of training data as input to the machine learning model(s) to generate an output prediction of injury risk (e.g., what injuries may occur, the probabilities of each such injury, and the like). This prediction can then be compared against the corresponding label(s) of one or more samples in order to generate a loss (e.g., using cross-entropy loss), and the loss can be used to update one or more model parameters (e.g., using backpropagation). This process may be performed iteratively for each training exemplar independently (e.g., using stochastic gradient descent) and/or in batches (e.g., using batch gradient descent).

In this way, the model can iteratively learn to predict the risk of injury caused by action performance (as indicated by the sensor data collected while the user performs the action).

At block 2620, the training system determines whether one or more training termination criteria have been met. The termination criteria may generally vary depending on the particular implementation. For example, in some embodiments, the training system may determine whether a defined period of time and/or amount of computing resources have been spent training the model(s). In some embodiments, the training system may determine whether the model(s) have reached a defined or preferred minimum accuracy. In some embodiments, the training system may determine whether a defined number of training iterations or epochs have been completed. In some embodiments, the training system may determine whether any additional training samples remain to be used.

If the training system determines that the termination criteria are not met, the method 2600 returns to block 2605 to access more (or new) training data. If, at block 2620, the training system determines that the termination criteria are met, the method 2600 continues to block 2625, where the training system deploys the machine learning model(s) to score injury risks during runtime. As discussed above, deploying the model(s) may generally include any operations used to prepare or provide the model to process input data (e.g., new sensor data) during runtime. For example, the training system may instantiate the model locally, or may transmit the model details (e.g., the values of the learned parameters) to one or more other systems for inferencing.

### Example Method for Training a Set of Machine Learning Models to Predict Relevant Injury Risks

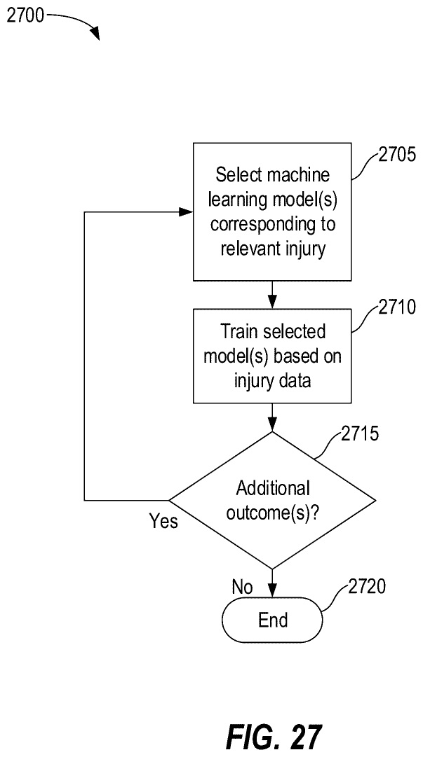

FIG. 27 is a flow diagram depicting an example method 2700 for training a set of machine learning models to predict relevant injury risks, according to some embodiments of the present disclosure. In some aspects, the method 2700 may be performed by a training system, such as the training system 220 of FIGS. 2-3, the training system 520 of FIGS. 5-7, the training system 920 of FIGS. 9-10, and/or the training systems discussed above with reference to FIGS. 12-14, 17-21, and/or 26. In some aspects, the method 2700 provides additional detail for block 2615 of FIG. 26.

At block 2705, the training system selects one or more machine learning models that correspond to the relevant injuries experienced by a user. That is, if the training system is training model(s) based on injury risks, the training system may determine which injuries the user actually experienced (e.g., indicated in the selected or current training exemplar), and may then select the corresponding model(s) that are being trained to predict this specific injury. In some embodiments, this may allow the training system to train injury-specific models (e.g., where each injury is predicted using a specific model trained based on data for that injury). This may result in substantially improved prediction accuracy, as each model may specialize and learn the specific correlations with respect to the specific injury.

In some embodiments, as discussed above, during inferencing the system may therefore process sensor data using a set of models (e.g., one for each potential injury) to generate a set of predictions. For example, the inferencing system may determine a set of relevant injury models (e.g., models that predict injuries which are relevant to or may be caused by the specific action that was performed, such as determined based on rules or mappings). By processing the sensor data using this set of models, the inferencing system can predict specific relevant injury risks in a more accurate and robust way.

At block 2710, the training system trains the selected model(s) based on the injury data indicated in the exemplar. That is, as discussed above, the training system trains one or more injury-specific models corresponding to the actual injury (or injuries) that occurred, using the sensor data as input and the actual injury as target output.

At block 2715, the training system determines whether there are one or more additional injuries, experienced by the user, which have not been used to train a corresponding model. If so, the method 2700 returns to block 2705 to select the model(s) corresponding to the one or more other injuries. If not, the method 2700 terminates at block 2720. As discussed above, these injury-specific models may be substantially more accurate, in some embodiments.

### Example Method for Generating Training Data for Machine Learning Models to Predict Action Injury Risk

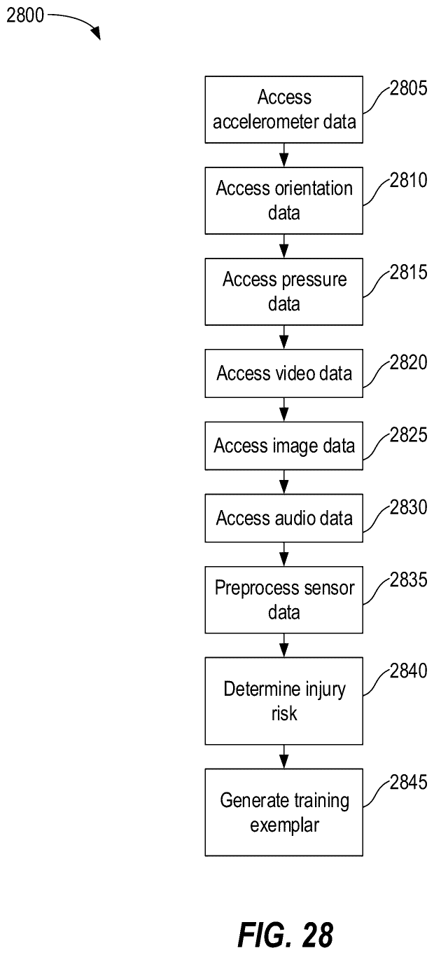

FIG. 28 is a flow diagram depicting an example method 2800 for generating training data for machine learning models to predict action injury risk, according to some embodiments of the present disclosure. In some aspects, the method 2800 may be performed by a training system, such as the training system 220 of FIGS. 2-3, the training system 520 of FIGS. 5-7, the training system 920 of FIGS. 9-10, and/or the training systems discussed above with reference to FIGS. 12-14, 17-21, and/or 26-27. In some aspects, the method 2800 provides additional detail for block 2605 of FIG. 26.

At block 2805, the training system accesses accelerometer data collected via one or more sensors as a user performs a task for a patient. For example, as discussed above, the accelerometer data may be collected via accelerometers embedded in one or more wearable devices, such as on the user's wrist(s), on the patient, and the like. In some aspects, the accelerometer data is collected from other devices or components, such as a smartphone of the user and/or patient, or one or more tools or devices used to provide the assistance (e.g., an accelerometer in a walker or cane used to help the patient walk).

At block 2810, the training system accesses orientation data collected via one or more orientation sensors as the user performs the task. In some embodiments, the orientation data may be collected from similar (or the same) devices to the accelerometer data. For example, the orientation data may be collected via orientation sensors embedded in one or more wearable devices, such as on the user's wrist(s), on the patient, and the like. In some aspects, the orientation data is collected from other devices or components, such as a smartphone of the user and/or patient, or one or more tools or devices used to provide the assistance (e.g., an orientation sensor in a walker or cane used to help the patient walk).

At block 2815, the training system accesses pressure data collected via one or more sensors during or after the user performs the task. For example, as discussed above the pressure data may be collected via pressure sensors to indicate information such as the grip strength of the user, the positioning of the patient, the amount of pressure exerted on the patient by the user, and the like.

At block 2820, the training system accesses video data depicting the user performing the action for the patient. For example, as discussed above, one or more video cameras may capture the performance from one or more angles in the space. In some embodiments, the video can include multiple types of video, such as color video for visible light (e.g., red, green, and blue (RGB)) video, video with depth information (e.g., RGB-Depth (RBG-D)) video, stereoscopic video, thermal video, and the like. In some embodiments, the user and/or patient may be prompted to capture the video data during or after performance of the action (e.g., to confirm that it was completed).

At block 2825, the training system accesses image data depicting the user performing the action for the patient. In some embodiments, the image data may be collected if video data is not available (e.g., due to insufficient bandwidth to transmit full video data, or because the room does not contain a video camera). For example, a video may be captured, and a subset of the frames from the video may be accessed as training data. For example, as discussed above, one or more video cameras may capture the performance from one or more angles in the space. In some embodiments, the image data can include multiple types of images, such as color images in the visible light spectrum, images with depth information, stereoscopic images, thermal images, and the like. In some embodiments, the user and/or patient may be prompted to capture the image data during or after performance of the action (e.g., to confirm that it was completed).

At block 2830, the training system accesses audio data of the user performing the action for the patient. For example, as discussed above, one or more microphones may capture the performance from one or more positions in the space. In some embodiments, the audio data includes audio in the audible frequency range. Generally, the audio data may include natural language speech (e.g., of the user and/or patient) as well as non-language audio (e.g., sounds caused by the action performance, such as a shower running, a pot boiling, a vacuum running, a toilet flushing, and the like). In some embodiments, the user and/or patient may be prompted to capture the audio data during or after performance of the action (e.g., to confirm that it was completed).

Although a number of data modalities (e.g., including accelerometer, orientation, pressure, video, image, and audio data) are depicted for conceptual clarity, these modalities are intended to be used as examples, and are not limiting on embodiments of the present disclosure. That is, the training system may use more or fewer modalities than those contemplated herein, and may generally use any relevant or useful sensor data.

At block 2835, the training system optionally preprocesses the sensor data if appropriate to render the data more suited for processing using machine learning models. Generally, the particular operations used for preprocessing may vary depending on the particular implementation and modality of the data. For example, the training system may remove outlier data, denoise the data, delineate the data into windows or points in time, aggregate the data, and the like.

At block 2840, the training system determines the injury risk of the performed action. For example, in some embodiments, the training system may determine whether the action did, in fact, result in an injury (e.g., whether the user performing the action sprained their back in the process). In some embodiments, the training system may determine the injury risk based on other criteria, such as based on “best practices” or instructions (e.g., indicating that certain forms of lifting are more likely to result in injury). In some embodiments, the training system may receive the injury risk information from another user, such as a user with expertise in the particular injury. Generally, a wide variety of operations may be used to determine or infer the injury risk, as discussed above.

At block 2845, the training system generates a training exemplar including the accessed sensor data (e.g., from each modality used) as the input portion of the exemplar, as well as the determined injury risk as the target output or label of the exemplar. In this way, the training exemplar may be stored and/or used to train or refine one or more injury machine learning models, as discussed above. In some aspects, the method 2800 may be repeated any number of times for any number of action performances of any number of actions by any number of users to assist any number of patients, as discussed above.

### Example Method for Refining Machine Learning Models to Predict Action Injury Risk for Specific Users

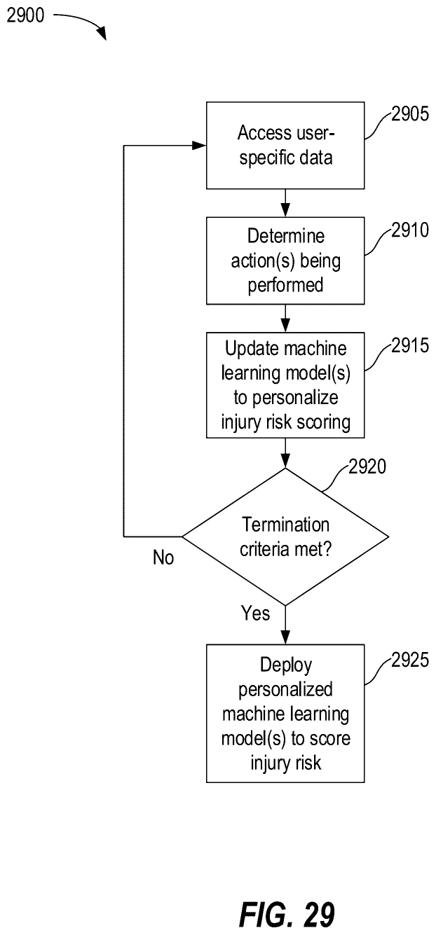

FIG. 29 is a flow diagram depicting an example method 2900 for refining machine learning models to predict action injury risk for specific users, according to some embodiments of the present disclosure. In some aspects, the method 2900 may be performed by a training system, such as the training system 220 of FIGS. 2-3, the training system 520 of FIGS. 5-7, the training system 920 of FIGS. 9-10, and/or the training systems discussed above with reference to FIGS. 12-14, 17-21, and/or 26-28. In some aspects, the method 2900 provides additional detail for the workflow 1000 of FIG. 10.

At block 2905, the training system accesses user-specific data. For example, as discussed above, the training system may access the sensor data associated with a particular user for whom a personalized model is being trained (e.g., the personalization data 1005 of FIG. 10). Generally, the user-specific data is said to be “associated with” the user to indicate that the user-specific data depicts or otherwise reflects the specific user performing action(s). For example, the user-specific data may include images and/or video depicted the user performing the action and/or depicting the patient after the action is performed (e.g., depicting bandaging), audio recording the user performing the action, accelerometer and/or orientation data from the user, and the like. In some embodiments, some or all of the user-specific data may also be associated with other users. For example, if two users simultaneously assist in lifting a patient, the video of the action may be user-specific data for both users. Similarly, in some embodiments, the user-specific data can be associated with the user performing any number and variety of actions for any number of patients.

At block 2910, the training system determines the action(s) being performed, as reflected by one or more exemplars in the user-specific data. For example, as discussed above, the training system may infer the action based on the movements or sensor data itself, or may identify the action based on manual or explicit labeling. For example, in some embodiments, the user may announce or record what actions they are performing (or did perform) to assist a patient. The training system may use this information to determine which action(s) each sample of training data correspond to.

At block 2915, the training system updates one or more machine learning models (e.g., the injury machine learning model 925 of FIG. 9) based on the user-specific data. Generally, updating a machine learning model (also referred to as fine-tuning or refining the model, in some embodiments) includes similar operations to the original training of the model. As discussed above, the particular operations and techniques used to update the machine learning model(s) may vary depending on the particular model architecture and implementation. For example, in the case of neural networks, the training system may process the sensor data of one or more samples of user-specific data as input to the machine learning model(s) to generate an output prediction of injury risk. This prediction can then be compared against a corresponding label(s) of one or more samples in order to generate a loss (e.g., using cross-entropy loss), and the loss can be used to update one or more model parameters (e.g., using backpropagation). This process may be performed iteratively for each user-specific exemplar independently (e.g., using stochastic gradient descent) and/or in batches (e.g., using batch gradient descent).

In some embodiments, the label for some or all of the user-specific data may be determined by the user themselves. For example, when the user is injured while performing an action, they may self-report that the injury and the action that was being performed (e.g., indicating that the pulled a muscle). This exemplar can then be used to refine their personalized model(s). Similarly, when the user does not feel any pain or injury, they may self-report that the action they just completed felt safe or unlikely to cause injury, and should not be used to train the model (or should be used as a negative exemplar, to personalize the model to identify non-injurious performance).

In some embodiments, the label for some or all of the user-specific data may be determined by other users, such as supervising users who observed or assisted with the action performance. For example, as discussed above, the supervising user may record a label indicating that the method of performance may result in injury.

Generally, the user-specific data may be tagged or labeled using any suitable technique or operation to allow the model to be personalized to the specific user. In some embodiments, prior to tagging or using user-specific data to refine the model, the training system may first confirm that the action was not performed in a way that may cause injury to the user. For example, if the (non-personalized) quality model indicates that the action was performed in a way that has a substantial risk of injury, it may not be relevant that the user felt it was done safely. Similarly, if another more experienced user reports that the action was performed in a risky way, it may be undesirable to refine the model using the performance as a positive example. In some aspects, therefore, the training system may determine whether the action was performed with at least a minimum level of safety (e.g., based on the prediction generated by the non-personalized model, or based on injury risk reported by individuals other than the user for whom the model is being personalized). If so, the data may be included in the user-specific data to personalize the model. If not, the training system may discard or otherwise refrain from using the data to personalize the model.

At block 2920, the training system determines whether one or more personalization termination criteria have been met. The termination criteria may generally vary depending on the particular implementation, as discussed above. For example, in some embodiments, the training system may determine whether a defined period of time and/or amount of computing resources have been spent personalizing the model(s). In some embodiments, the training system may determine whether a defined number of personalization iterations or epochs have been completed. In some embodiments, the training system may determine whether any additional user-specific samples remain to be used.

If the training system determines that the termination criteria are not met, the method 2900 returns to block 2905 to access more (or new) user-specific data. If, at block 2920, the training system determines that the termination criteria are met, the method 2900 continues to block 2925, where the training system deploys the machine learning model(s) to score injury risk during runtime for the specific user. That is, when sensor data for the specific user is received during runtime, the training system may use the personalized model (e.g., the refined injury machine learning model 1025 of FIG. 10) to process the sensor data, rather than using the non-personalized model.

### Example Method for Using Machine Learning Models to Predict Action Injury Risk

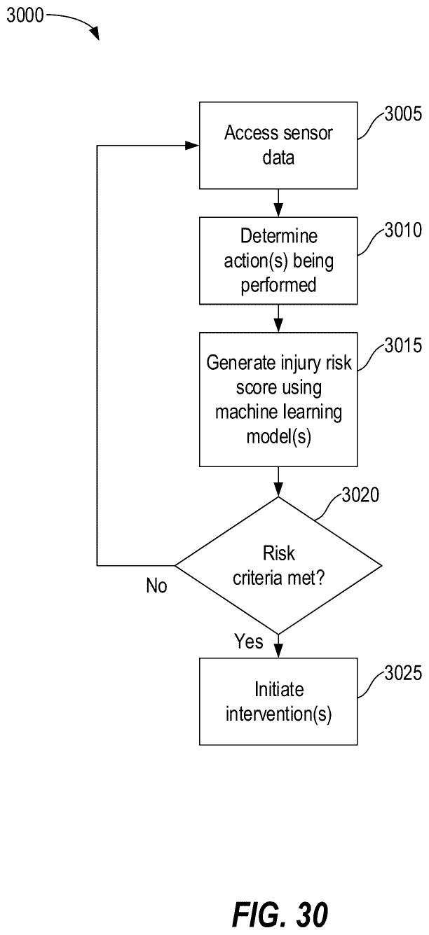

FIG. 30 is a flow diagram depicting an example method 3000 for using machine learning models to predict action injury risk, according to some embodiments of the present disclosure. In some aspects, the method 3000 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16, and/or 22-25. In some aspects, the method 3000 provides additional detail for the workflow 1100 of FIG. 11.

At block 3005, the monitoring system accesses sensor data collected while a user performs an action to assist a patient. For example, as discussed above, the monitoring system may access sensor data such as accelerometer data, orientation data, video data, image data, audio data, pressure data, and the like. In some aspects, as discussed above, the monitoring system accesses the sensor data in response to determining that the user is performing (or just completed performance of) the action. Generally, the sensor data (which may correspond to the sensor data 1105 of FIG. 11) may comprise any relevant data that may be useful to predict how risky the performance of the action was (e.g., how likely the performance is to result in injury to the user).

At block 3010, the monitoring system determines what action(s) were being performed when the sensor data was recorded. For example, as discussed above, the monitoring system may infer the action based on the movements or sensor data itself, or may identify the action based on manual or explicit labeling. For example, in some embodiments, the user may announce or record what actions they are performing (or did perform) to assist a patient. The monitoring system may use this information to determine which action(s) the sensor data corresponds to. In some embodiments, the user may use their personal device before, during and/or after performing the action to indicate its performance (e.g., to indicate the start time, end time, or any other characteristics of the action).

At block 3015, the monitoring system generates one or more injury risk scores (e.g., the injury score 1115 of FIG. 11) by processing some or all of the sensor data (accessed at block 3005) using one or more injury machine learning models (e.g., the injury machine learning model 925 of FIG. 11, and/or a personalized version of the injury machine learning model, as discussed above). In some embodiments, the injury score is generally a set of one or more numerical values (e.g., between 0 and 1, or between 0 and 100) indicating the probability that one or more injuries will be caused by the way in which the user performed the action (e.g., where higher scores indicate higher likelihood).

At block 3020, the monitoring system determines whether the injury score(s) satisfy one or more defined criteria. For example, the monitoring system may compare the injury score(s) against one or more thresholds to determine whether the injury risk of the performance was above or below the defined level(s) of risk. In some embodiments, in addition to or instead of evaluating the current performance alone, the monitoring system may evaluate the user's risk scores over time. For example, the monitoring system may determine whether the injury risk scores are trending upwards or downwards, whether the injury risk scores have remained above or below one or more thresholds for a threshold period of time (or a threshold number of performances), and the like.

If the criteria are not met (e.g., the action performance was sufficiently non-risky), the method 3000 returns to block 3005. If the criteria are met (e.g., the action performance was sufficiently likely to result in one or more injuries to the user), the method 3000 proceeds to block 3025. At block 3025, the monitoring system initiates one or more interventions for the user based on the risk. For example, as discussed above, the monitoring system may notify the user, the patient, a supervisor, and the like.

In this way, the monitoring system can use machine learning to monitor user performance and ensure that user is not injured.

In some embodiments, the method 3000 is performed at least in part in response to determining that the user has already suffered a prior injury and is in an injury recovery state (e.g., whether they are currently recovering from, or recently recovered from, an injury, such as if they are working a return to work shift). For example, suppose a user suffers an injury (either in connection with their work, or outside of work) and may or may not take some amount of time off work to recover. In some embodiments, the user may be considered to be in an “injury recovery” state for some period of time after the injury, such as for a certain number of days (which may be fixed or may vary depending on the particular injury and/or how the patient is recovering), a certain number of shifts worked without further injury, and the like. In some embodiments, the first shift that a user works after an injury (or the first N shifts, such as all shifts worked during the “injury recovery” state) may be referred to as a “return to work” shift to indicate that the user has either just recovered or may be in the process of recovering from an injury. Based on this determination, The monitoring system may infer that the user may be more susceptible to further injury than other users, and may therefore determine to use the method 3000 to evaluate injury risk (also referred to as re-injury risk in some aspects) for the user. For users without such injury history, the monitoring system may determine that the computational expense of predicting injury risk is not justified, as the user may be at a low chance of injury given their history. By dynamically using the method 3000 only when users are returning to work, the monitoring system may be able to substantially reduce the computational expense (e.g., power consumption, memory usage, compute time, and the like) of the system.

In some embodiments, when using the method 3000 to evaluate injury risk for a user that was previously injured, the monitoring system may specifically evaluate the re-injury risk for the same (or related) injuries. For example, if the user is working a return to work shift after suffering a leg injury, the monitoring system may specifically evaluate the risk of the user re-injuring their leg, or causing another injury because their leg is injured (e.g., injuring their other leg or their back, because they are trying to keep weight off the injured leg).

### Example Method for Using a Set of Machine Learning Models to Predict Relevant User Injury Risks

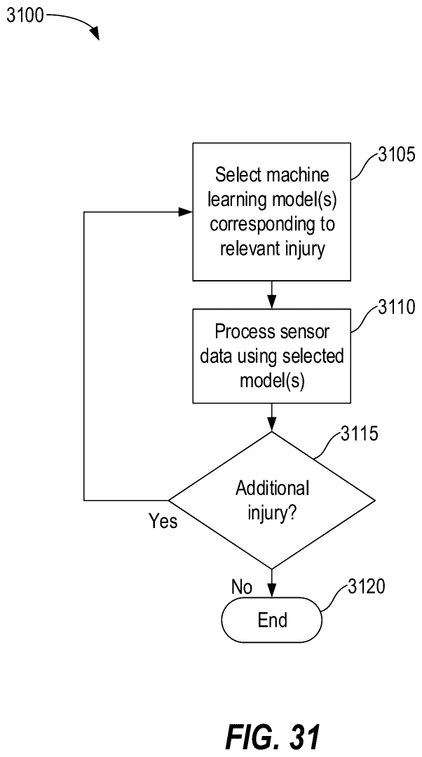

FIG. 31 is a flow diagram depicting an example method 3100 for using a set of machine learning models to predict relevant user injury risks, according to some embodiments of the present disclosure. In some aspects, the method 3100 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16, 22-25, and/or 30. In some aspects, the method 3100 provides additional detail for block 3015 of FIG. 30.

At block 3105, the monitoring system selects one or more machine learning models that correspond to the relevant injury or injuries for which the monitoring system is generating a prediction. That is, the monitoring system may determine which injuries the user will potentially experience as a result of the action performance, and may then select the corresponding model(s) that are trained to predict these specific injuries. For example, as discussed above, the monitoring system may evaluate one or more mappings indicating what injuries may be caused by various action(s). As another example, the monitoring system may evaluate any prior injuries suffered by the user, and select the model(s) that are trained to predict the risk of such injury re-occurring and/or to predict the risk of related injuries. Based on the action(s) that were performed (or are being performed) and/or the prior injuries that have occurred, therefore, the monitoring system may identify the relevant injury model(s).

At block 3110, the monitoring system processes the sensor data using the selected model(s) to generate a set of one or more injury risk score(s) indicating the predicted probability that each injury will occur, based on the action performance.

At block 3115, the monitoring system determines whether there are one or more additional injuries, which may be experienced by the user, which have not been evaluated. If so, the method 3100 returns to block 3105 to select the model(s) corresponding to the one or more other injuries. If not, the method 3100 terminates at block 3120. As discussed above, these injury-specific models may be substantially more accurate, in some embodiments.

### Example Method for Generating Interventions Based on Machine Learning Evaluations of Action Injury Risk

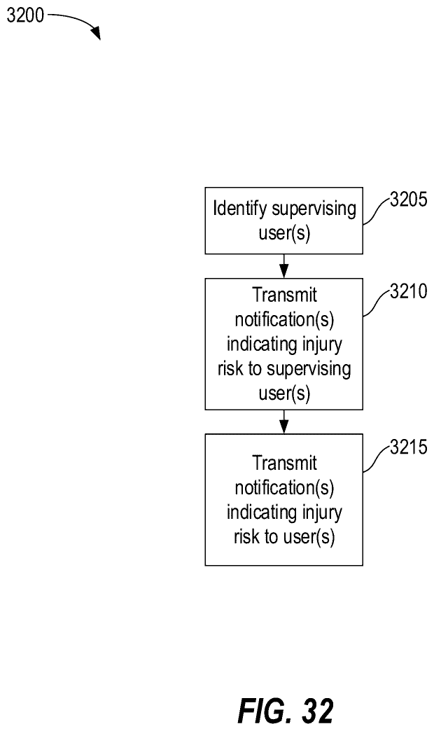

FIG. 32 is a flow diagram depicting an example method 3200 for generating interventions based on machine learning evaluations of action injury risk, according to some embodiments of the present disclosure. In some aspects, the method 3200 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16, 22-25, and/or 30-31. In some aspects, the method 3200 provides additional detail for block 3025 of FIG. 30.

At block 3205, the monitoring system optionally identifies one or more supervising users who are responsible for supervising or otherwise managing the user that performed (or is performing) the action. For example, the monitoring system may evaluate one or more mappings or other data to identify users to whom the acting user reports.

At block 3210, the monitoring system optionally transmits one or more notifications to the supervising user(s). For example, as discussed above, the notification may indicate what action was (or is) being performed, what user performed (or is performing) it, what patient was (or is) being assisted, when the action was performed, where the action was performed, and the like. In some embodiments, the notification may include further information such as some or all of the sensor data (e.g., imagery or video), the generated injury risk score(s), and the like. This may allow a supervising user to check in with the acting user in order to ensure they are not re-injured. For example, the supervising user may determine that the acting user should take more time to recover before performing the specific action, or that the user should take additional precautions when performing the specific action.

At block 3215, the monitoring system transmits one or more notifications to the user that performed (or is performing) the action. For example, the monitoring system may notify the user that the action (or the way the action was performed, such as the position(s) the user was in while performing the action) may cause the predicted injuries to occur. In some embodiments, these notifications are provided in real-time (or near real-time), such as while the user is still performing the action. For example, the user's smartphone or wearable may vibrate, ring, or otherwise output a notification or alert to the user, allowing them to immediately review the information to determine how to proceed.

For example, the notification may inform the user that they are standing in such a way that, if they proceed with lifting the patient, they are likely to strain their back. In response, the user may reposition themselves to lift more safely. As another example, the notification may inform the user that the way they are holding their hands may cause wrist strain over time. In response, the user may use a different grip orientation.

Generally, each intervention discussed in the present disclosure may be an optional response to the potential outcomes. That is, the actions discussed herein are intended to be used as examples, and are not limiting on aspects of the present disclosure. In embodiments, the monitoring system may initiate more or fewer interventions, and may generally take any appropriate action in response to the potential injuries.

### Example Method for Training Action-Specific Machine Learning Models

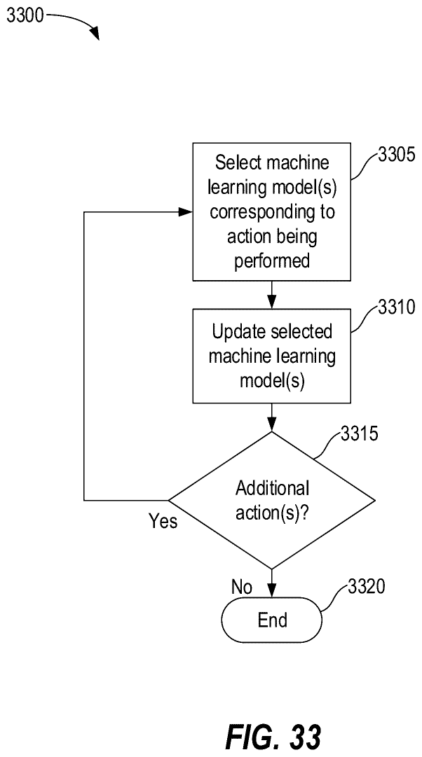

FIG. 33 is a flow diagram depicting an example method 3300 for training action-specific machine learning models, according to some embodiments of the present disclosure. In some aspects, the method 3300 may be performed by a training system, such as the training system 220 of FIGS. 2-3, the training system 520 of FIGS. 5-7, the training system 920 of FIGS. 9-10, and/or the training systems discussed above with reference to FIGS. 12-14, 17-21, and/or 26-29. In some aspects, the method 3300 provides additional detail for block 1215 of FIG. 12, block 1415 of FIG. 14, block 1715 of FIG. 17, block 1905 of FIG. 19, block 2615 of FIG. 26, block 2710 of FIG. 27, and/or block 2915 of FIG. 29. Generally, the method 3300 may be used to train action-specific models for any model discussed above that is trained to generate predictions based on action performance.

At block 3305, the training system selects one or more machine learning models that correspond to an action that is currently being performed by a user (or the action that was being performed when the training data was collected). This may include, for example, when the training system is training models to predict action performance quality, when the training system is training models to predict patient positions and/or outcomes based on action performance, and/or when the training system is training models to predict injury risk based on action performance. In some embodiments, training action-specific models (e.g., where each action performance is evaluated using a specific model trained based on data for that specific action) may result in substantially improved prediction accuracy, as each model may specialize and learn the specific correlations with respect to the specific actions.

At block 3310, the training system trains or updates the selected model(s) based on the sensor data indicated in the exemplar. That is, as discussed above, the training system trains one or more action-specific models corresponding to the specific action(s) being performed. In some embodiments, in addition to input-specific models (where the model is selected based on what the input data corresponds to, such as what action is being performed), the training system can further select and train output-specific models (where the model is trained based on what the desired output corresponds to, such as what type of injury is being predicted, what outcome(s) are being predicted, and the like). That is, the training system may train a first model to predict the probability of a first outcome when a first action is performed, a second model to predict the probability of the first outcome when a second action is performed, a third model to predict the probability of a second outcome when the first action is performed, a fourth model to predict the probability of the second outcome when the second action is performed, and so on.

At block 3315, the training system determines whether there are one or more additional action(s), reflected in the training data, which have not been used to train a corresponding model. If so, the method 3300 returns to block 3305 to select the model(s) corresponding to the one or more other action(s). If not, the method 3300 terminates at block 3320. As discussed above, these action-specific models may be substantially more accurate, in some embodiments.

### Example Method for Using Action-Specific Machine Learning Models

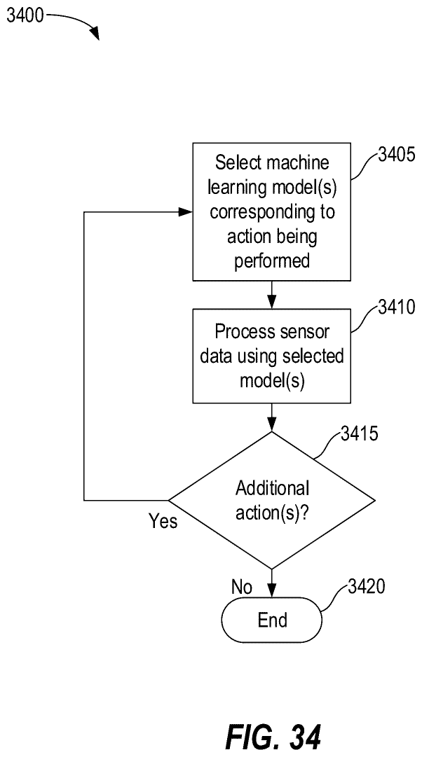

FIG. 34 is a flow diagram depicting an example method 3400 for using action-specific machine learning models, according to some embodiments of the present disclosure. In some aspects, the method 3400 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16, 22-25, and/or 30-32. In some aspects, the method 3400 provides additional detail for block 1515 of FIG. 15, block 2215 of FIG. 22, block 2315 of FIG. 23, block 3015 of FIG. 30, and/or block 3115 of FIG. 31. Generally, the method 3400 may be used to utilize action-specific models for any prediction discussed above where predictions are generated based on action performance.

At block 3405, the monitoring system selects one or more machine learning models that correspond to the relevant action(s) for which the monitoring system is generating a prediction. That is, the monitoring system may determine which action(s) the user is performing (or was performing when the sensor data was collected), and may then select the corresponding model(s) that are trained to predict output this specific action. For example, as discussed above, the user may specify what action(s) they are (or were) performing.

At block 3410, the monitoring system processes the sensor data using the selected model(s) to generate a set of one or more outputs for the action performance. For example, as discussed above, the monitoring system may predict the quality of the action performance, the probability of one or more outcomes for the patient, the probability of one or more injuries to the user, and the like.

At block 3415, the monitoring system determines whether there are one or more additional action(s), which are being or were performed by the user, which have not been evaluated. If so, the method 3300 returns to block 3405 to select the model(s) corresponding to the one or more other action(s). If not, the method 3400 terminates at block 3420. As discussed above, these action-specific models may be substantially more accurate, in some embodiments.

### Example Method for Accessing Sensor Data for Machine Learning

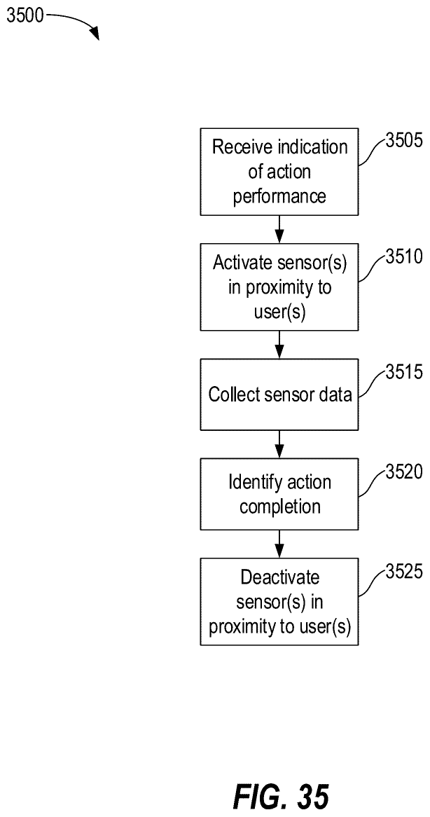

FIG. 35 is a flow diagram depicting an example method 3500 for accessing sensor data for machine learning, according to some embodiments of the present disclosure. In some aspects, the method 3500 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16, 22-25, 30-32, and/or 34. In some aspects, the method 3500 provides additional detail for the method 1300 of FIG. 13, the method 1800 of FIG. 18, and/or the method 2800 of FIG. 28. In some aspects, the method 3500 provides additional detail for block 1405 of FIG. 14, 1505 of FIG. 15, block 2205 of FIG. 22, block 2905 of FIG. 29, block 3005 of FIG. 30. Generally, the method 3500 may be used any time sensor data is captured, such as during runtime inferencing and/or while collecting training data.

At block 3505, the monitoring system receives an indication of action performance. As discussed above, the indication of action performance may generally include any indication or determination that one or more users are performing, have performed, or are about to perform one or more actions to assist one or more patients. For example, in some embodiments, the monitoring system may receive an explicit input from the user, such as via a verbal cue, a hand gesture, an interaction with a user interface on a computing device (e.g., pressing a button on a tablet, smartphone, or wearable), and the like. In some embodiments, the notification may specify the information such as the identity of the user(s), the identity of the patient(s), the location of the user(s) and patient(s) (e.g., what room number they are in, in a residential care facility), the action(s) being performed, and the like.

In some embodiments, the monitoring system (or another system) may determine or infer the action performance based on evaluating sensor data. For example, the monitoring system (or another system) may evaluate sensor to determine whether the defined cues or inputs have been provided to indicate that an action is occurring. If not, the sensor data may be discarded for privacy and to reduce computational expense and memory overhead. If the cue or inputs are indicated, the monitoring system may continue to collect sensor data for full evaluation. As discussed above, these cues may include inputs such as verbally stating that the action is beginning, or may be inferred (e.g., based on evaluating image data, the monitoring system may determine that the user is preparing to lift the patient).

At block 3510, the monitoring system activates one or more sensors in physical proximity to the user(s) performing the action(s). For example, as discussed above, the monitoring system may identify which device(s) (e.g., wearable sensors) are associated with the specific user(s) that are performing the action, and may activate such sensors. As another example, the monitoring system may identify which device(s) (e.g., cameras, pressure sensors, and the like) are present in the room where the action is being performed, and may activate those sensors. In some embodiments, as discussed above, prior to being activated the monitoring system may be in an inactive or deactivated state. In this state, as discussed above, the sensors may generally not collect sensor data (e.g., if they are powered down or have a physical shutter blocking the sensor), not record or save sensor data, not transmit the sensor data, not evaluate the sensor data, and the like.

In some embodiments, notwithstanding the inactive or deactivated status of the sensor(s), some or all of the sensors may collect and evaluate a relatively constrained set of data to determine whether to proceed with full data collection and evaluation. For example, as discussed above, a microphone associated with a user may monitor small snippets of speech to determine whether the user is about to perform an action. When the action is detected, the monitoring system may activate additional sensors to collect the relevant sensor data.

At block 3515, the monitoring system collects sensor data using the one or more sensors (which may include sensors activated at block 3510, and/or one or more sensors that were already activated) during the performance of the action(s). Generally, as discussed above, the particular data collected may vary depending on the particular implementation, and may include information such as accelerometer data, orientation data, pressure data, video data, and the like.

At block 3520, the monitoring system identifies action completion. In some embodiments, as discussed above, the monitoring system identifies action completion in a similar manner to identifying the beginning of the performance. For example, the user may specify or indicate explicitly that they have completed the action (e.g., verbally, by interacting with a user interface, and the like). As another example, the monitoring system may infer action completion by evaluating the sensor data (e.g., detecting that the user has stepped away from or left the proximity of the patient, detecting that the expected outcome is reflected in the sensor data, such as that the patient has been moved to the intended location, and the like).

At block 3525, the monitoring system deactivates the one or more sensor(s) in proximity to the user which were activated at block 3510. As discussed above, deactivating the sensor(s) generally corresponds to any actions used to return the sensors to an inactive state, such as by turning off the sensors, instructing the sensors to refrain from further data collection, transmission, and/or evaluation, operating physical shutters or other physical components to shield or block the sensors, and the like.

In this way, patient privacy may be maintained because sensor data may only be collected and/or saved when a user is actively providing assistance to the patient. Further, using the method 3500, the computational expense on the system may be substantially reduced. For example, network bandwidth may be preserved by refraining from transmitting data from all sensors at all times. Similarly, memory or storage footprint are substantially reduced. Additionally, power consumption (of the sensors, the monitoring system, and any other involved systems) may be reduced. Further, in some embodiments, the life of the sensor(s) may be expanded, as they may operate on a substantially reduced duty cycle as compared to continuous usage.

### Example Method for Learning to Predict Action Performance Quality

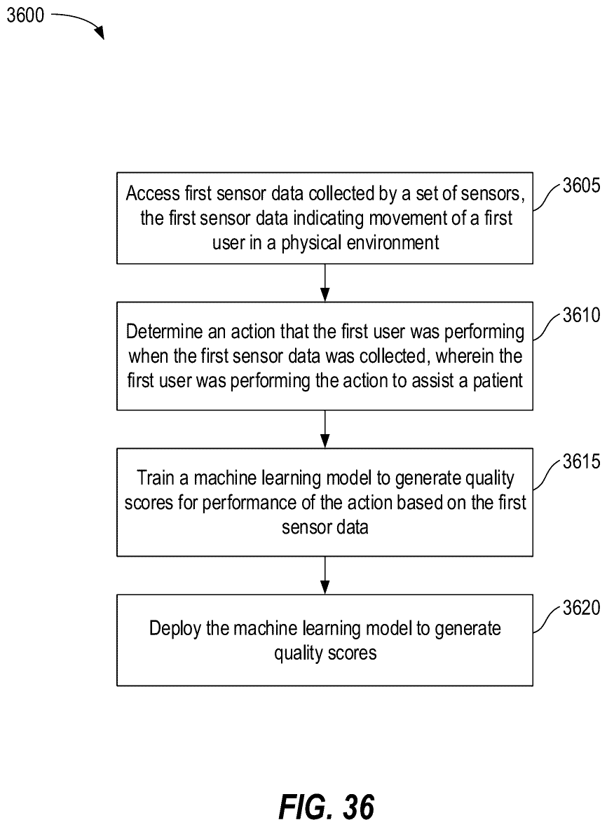

FIG. 36 is a flow diagram depicting an example method 3600 for learning to predict action performance quality, according to some embodiments of the present disclosure. In some aspects, the method 3800 may be performed by a training system, such as the training system 220 of FIGS. 2-3, the training system 520 of FIGS. 5-7, the training system 920 of FIGS. 9-10, and/or the training systems discussed above with reference to FIGS. 12-14, 17-21, 26-29, and/or 33.

At block 3605, first sensor data (e.g., the sensor data 120 of FIG. 1, the sensor data 210 of FIG. 2, and/or the sensor data 310 of FIG. 3) collected by a set of sensors (e.g., the sensors 117 of FIG. 1) is accessed, the first sensor data indicating movement of a first user (e.g., the user 110 of FIG. 1) in a physical environment.

At block 3610, an action that the first user was performing when the first sensor data was collected is determined, wherein the first user was performing the action to assist a patient (e.g., the patient 115 of FIG. 1).

At block 3615, a machine learning model (e.g., the quality machine learning model 225 of FIGS. 2-4 and/or the refined quality machine learning model 325 of FIG. 3) is trained to generate quality scores for performance of the action based on the first sensor data.

At block 3620, the machine learning model is deployed to generate quality scores (e.g., the quality score 415 of FIG. 4).

### Example Method for Predicting Action Performance Quality

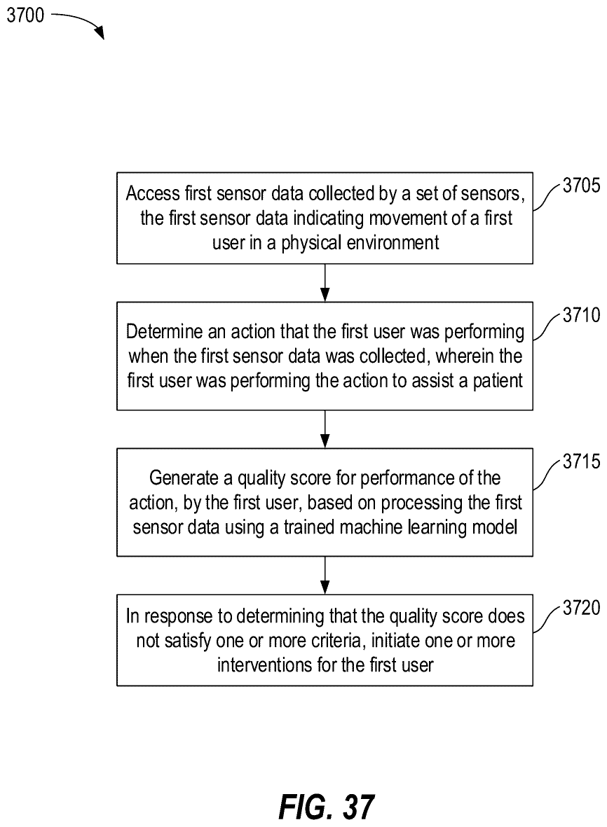

FIG. 37 is a flow diagram depicting an example method 3700 for predicting action performance quality, according to some embodiments of the present disclosure. In some aspects, the method 3700 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16, 22-25, 30-32, and/or 34-35.

At block 3705, first sensor data (e.g., the sensor data 120 of FIG. 1 and/or the sensor data 405 of FIG. 4) collected by a set of sensors (e.g., the sensors 117 of FIG. 1) is accessed, the first sensor data indicating movement of a first user (e.g., the user 110 of FIG. 1) in a physical environment.

At block 3710, an action that the first user was performing when the first sensor data was collected is determined, wherein the first user was performing the action to assist a patient (e.g., the patient 115 of FIG. 1).

At block 3715, a quality score (e.g., the quality score 415 of FIG. 4) is generated for performance of the action, by the first user, based on processing the first sensor data using a trained machine learning model e.g., the quality machine learning model 225 of FIGS. 2-4 and/or the refined quality machine learning model 325 of FIG. 3.

At block 3720, in response to determining that the quality score does not satisfy one or more criteria, one or more interventions (e.g., the interventions 425 of FIG. 4) are initiated for the first user.

### Example Method for Learning to Predict Patient Outcomes Based on Sensor Data

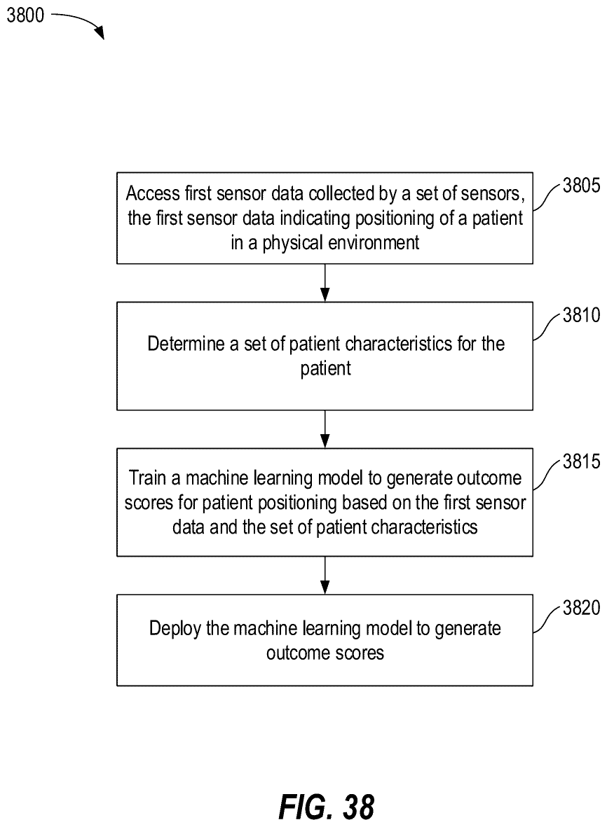

FIG. 38 is a flow diagram depicting an example method 3800 for learning to predict patient outcomes based on sensor data, according to some embodiments of the present disclosure. In some aspects, the method 3800 may be performed by a training system, such as the training system 220 of FIGS. 2-3, the training system 520 of FIGS. 5-7, the training system 920 of FIGS. 9-10, and/or the training systems discussed above with reference to FIGS. 12-14, 17-21, 26-29, 33, and/or 36.

At block 3805, first sensor data (e.g., the sensor data 120 of FIG. 1, the sensor data 510 of FIG. 5, the sensor data 610 of FIG. 6, and/or the sensor data 710 of FIG. 7) collected by a set of sensors (e.g., the sensors 117 of FIG. 1) is accessed, the first sensor data indicating positioning of a patient (e.g., the patient 115 of FIG. 1) in a physical environment.

At block 3810, a set of patient characteristics (e.g., the patient database 130 of FIG. 1 and/or patient data 807 of FIG. 8) for the patient is determined.

At block 3815, a machine learning model (e.g., the outcome machine learning model 525 of FIGS. 5-8 and/or the refined outcome machine learning model 725 of FIG. 7) is trained to generate outcome scores for patient positioning based on the first sensor data and the set of patient characteristics.

At block 3820, the machine learning model is deployed to generate outcome scores (e.g., the outcome score 815 of FIG. 8).

### Example Method for Predicting Patient Outcomes Based on Sensor Data

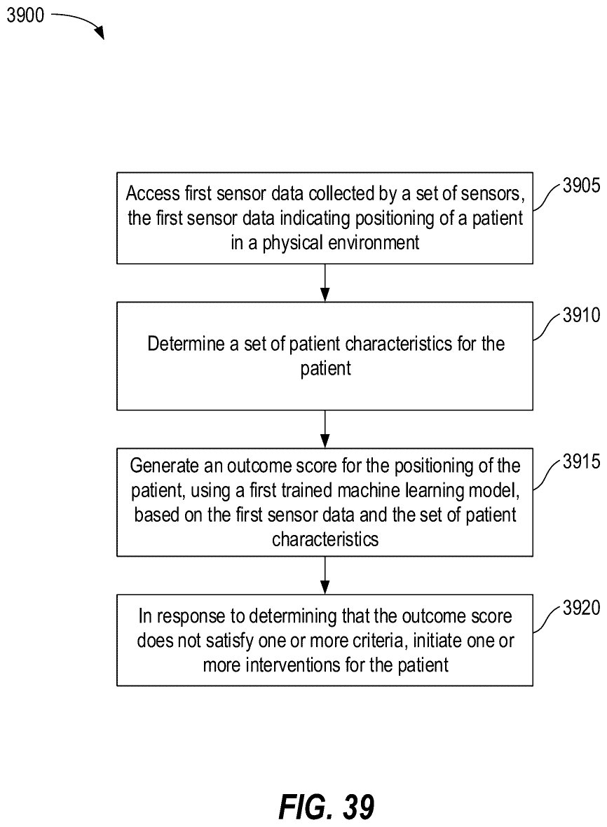

FIG. 39 is a flow diagram depicting an example method 3900 for predicting patient outcomes based on sensor data, according to some embodiments of the present disclosure. In some aspects, the method 3900 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16, 22-25, 30-32, 34-35, and/or 37.

At block 3905, first sensor data (e.g., the sensor data 120 of FIG. 1 and/or the sensor data 805 of FIG. 8) collected by a set of sensors (e.g., the sensors 117 of FIG. 1) is accessed, the first sensor data indicating positioning of a patient (e.g., the patient 115 of FIG. 1) in a physical environment.

At block 3910, a set of patient characteristics (e.g., the patient database 130 of FIG. 1 and/or patient data 807 of FIG. 8) for the patient is determined.

At block 3915, an outcome score (e.g., the outcome score 815 of FIG. 8) for the positioning of the patient is generated, using a first trained machine learning model (e.g., the outcome machine learning model 525 of FIGS. 5-8 and/or the refined outcome machine learning model 725 of FIG. 7), based on the first sensor data and the set of patient characteristics.

At block 3920, in response to determining that the outcome score does not satisfy one or more criteria, one or more interventions (e.g., the interventions 825 of FIG. 8) are initiated for the patient.

### Example Method for Learning to Predict Action Injury Risk

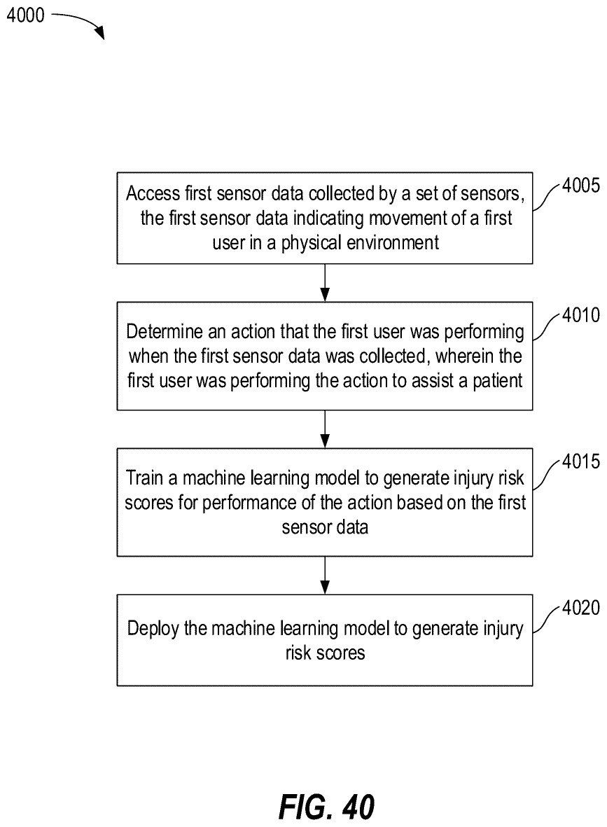

FIG. 40 is a flow diagram depicting an example method 4000 for learning to predict action injury risk, according to some embodiments of the present disclosure. In some aspects, the method 4000 may be performed by a training system, such as the training system 220 of FIGS. 2-3, the training system 520 of FIGS. 5-7, the training system 920 of FIGS. 9-10, and/or the training systems discussed above with reference to FIGS. 12-14, 17-21, 26-29, 33, 36, and/or 38.

At block 4005, first sensor data (e.g., the sensor data 120 of FIG. 1, the sensor data 910 of FIG. 9 and/or the sensor data 1010 of FIG. 10) collected by a set of sensors (e.g., the sensors 117 of FIG. 1) is accessed, the first sensor data indicating movement of a first user (e.g., the user 110 of FIG. 1) in a physical environment.

At block 4010, an action that the first user was performing when the first sensor data was collected is determined, wherein the first user was performing the action to assist a patient (e.g., the patient 115 of FIG. 1).

At block 4015, a machine learning model (e.g., the injury machine learning model 925 of FIGS. 9-11 and/or the injury machine learning model 1025 of FIG. 10) is trained to generate injury risk scores for performance of the action based on the first sensor data.

At block 4020, the machine learning model is deployed to generate injury risk scores (e.g., the injury score 1115 of FIG. 11).

### Example Method for Predicting Action Injury Risk

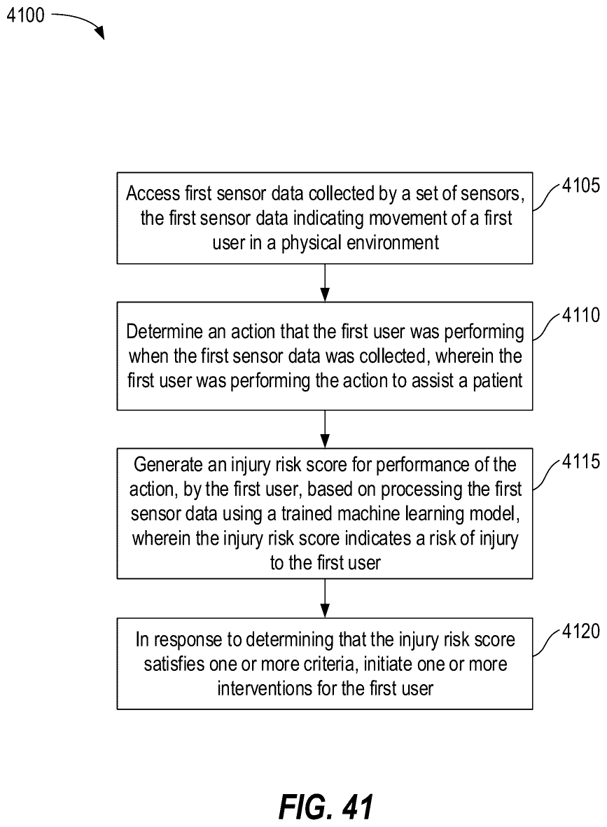

FIG. 41 is a flow diagram depicting an example method 4100 for predicting action injury risk, according to some embodiments of the present disclosure. In some aspects, the method 4100 may be performed by a monitoring system, such as the monitoring system 105 of FIG. 1, the inference component 410 and/or intervention component 420 of FIG. 4, the inference component 810 and/or intervention component 820 of FIG. 8, the inference component 1110 and/or intervention component 1120 of FIG. 11, and/or the monitoring systems discussed above with reference to FIGS. 15-16, 22-25, 30-32, 34-35, 37 and/or 39.

At block 4105, first sensor data (e.g., the sensor data 120 of FIG. 1 and/or the sensor data 1105 of FIG. 11) collected by a set of sensors (e.g., the sensors 117 of FIG. 1) is accessed, the first sensor data indicating movement of a first user (e.g., the user 110 of FIG. 1) in a physical environment.

At block 4110, an action that the first user was performing when the first sensor data was collected is determined, wherein the first user was performing the action to assist a patient (e.g., the patient 115 of FIG. 1).

At block 4115, an injury risk score (e.g., the injury score 1115 of FIG. 11) for performance of the action is generated, by the first user, based on processing the first sensor data using a trained machine learning model (e.g., the injury machine learning model 925 of FIGS. 9-11 and/or the injury machine learning model 1025 of FIG. 10), wherein the injury risk score indicates a risk of injury to the first user.

At block 4120, in response to determining that the injury risk score satisfies one or more criteria, one or more interventions (e.g., the interventions 1125 of FIG. 11) are initiated for the first user.

### Example Computing System for Improved Action Performance Prediction

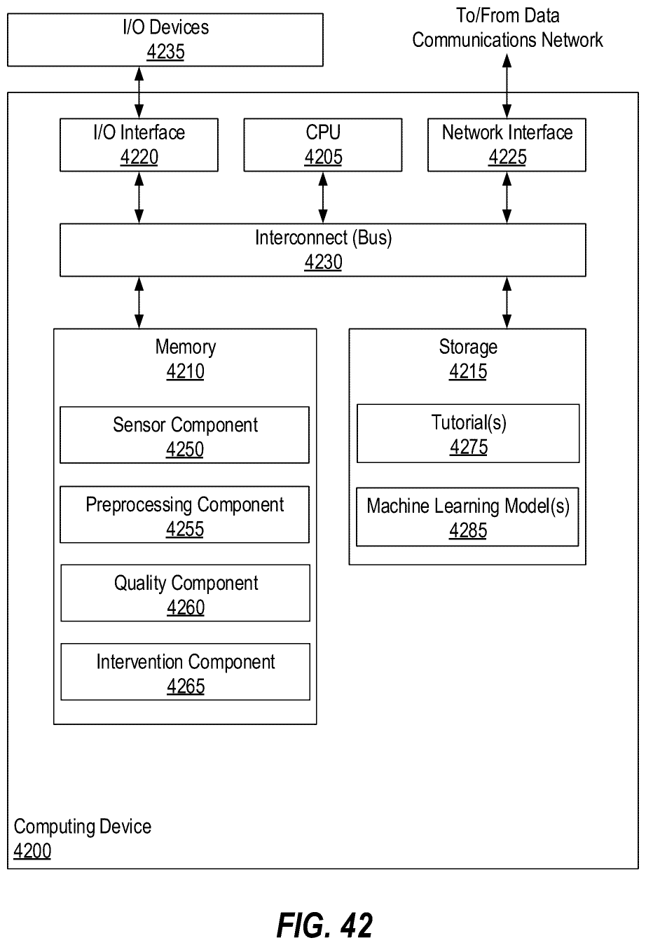

FIG. 42 depicts an example computing device 4200 configured to perform various aspects of the present disclosure. Although depicted as a physical device, in embodiments, the computing device 4200 may be implemented using virtual device(s), and/or across a number of devices (e.g., in a cloud environment). In one embodiment, the computing device 4200 corresponds to one or more systems that train and/or use machine learning models to predict action performance quality, such as the monitoring system 105 of FIG. 1, the training system 220 of FIGS. 2-3, the inference component 410 and/or intervention component 420 of FIG. 4, and the like.

As illustrated, the computing device 4200 includes a CPU 4205, memory 4210, storage 4215, a network interface 4225, and one or more I/O interfaces 4220. In the illustrated embodiment, the CPU 4205 retrieves and executes programming instructions stored in memory 4210, as well as stores and retrieves application data residing in storage 4215. The CPU 4205 is generally representative of a single CPU and/or GPU, multiple CPUs and/or GPUs, a single CPU and/or GPU having multiple processing cores, and the like. The memory 4210 is generally included to be representative of a random access memory. Storage 4215 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 4235 (such as keyboards, monitors, etc.) are connected via the I/O interface(s) 4220. Further, via the network interface 4225, the computing device 4200 can be communicatively coupled with one or more other devices and components (e.g., via a network, which may include the Internet, local network(s), and the like). As illustrated, the CPU 4205, memory 4210, storage 4215, network interface(s) 4225, and I/O interface(s) 4220 are communicatively coupled by one or more buses 4230.

In the illustrated embodiment, the memory 4210 includes a sensor component 4250, a preprocessing component 4255, a quality component 4260, and an intervention component 4265, which may perform one or more embodiments discussed above. Although depicted as discrete components for conceptual clarity, in embodiments, the operations of the depicted components (and others not illustrated) may be combined or distributed across any number of components. Further, although depicted as software residing in memory 4210, in embodiments, the operations of the depicted components (and others not illustrated) may be implemented using hardware, software, or a combination of hardware and software.

In some embodiments, the sensor component 4250 may be used to manage sensors, such as to activate and deactivate them, as discussed above. For example, the sensor component 4250 may be used to dynamically activate sensors when actions are being performed, and facilitate collection and/or storage of the sensor data. After action completion, the sensor component 4250 may selectively deactivate some or all of the sensors to preserve patient privacy and reduce computational expense, as discussed above.

In some embodiments, the preprocessing component 4255 may be used to process or preprocess sensor data, as discussed above. For example, the preprocessing component 4255 may be used to remove noise, smooth the data, delineate the data into windows of time, determine which data to collect or analyze, and the like.

In some embodiments, the quality component 4260 may be used to predict the quality of action performance, as discussed above. For example, the quality component 4260 may use machine learning models (e.g., the quality machine learning model 225 of FIGS. 2-4, the refined quality machine learning model 325 of FIG. 3, and/or the machine learning model(s) 4285) to process sensor data in order to generate score(s) or other output indicating how well an action was performed (e.g., how closely it mirrors an exemplar or instructed technique).

In some embodiments, the intervention component 4265 may be used to generate or facilitate interventions, as discussed above. For example, the intervention component 4265 may be used to evaluate the predicted action quality using one or more criteria, and to initiate interventions such as notifying the user when the criteria are not satisfied.

In the illustrated example, the storage 4215 includes tutorial(s) 4275 and machine learning model(s) 4285. In one embodiment, the tutorial(s) 4275 may include various pieces of content (e.g., video, audio, written materials, images, and the like) that may be used to instruct or teach a user how to perform various assistive actions, as discussed above. In some embodiments, the intervention component 4265 may select content from the tutorial(s) 4275 to send to users, based on the action quality. The machine learning model(s) 4285 generally correspond to one or more machine learning models that generate predicted quality scores or measures based on input sensor data (e.g., the quality machine learning model 225 of FIGS. 2-4 and/or the refined quality machine learning model 325 of FIG. 3). Although depicted as residing in storage 4215, the tutorials 4275 and the machine learning models 4285 may be stored in any suitable location, including memory 4210.

### Example Computing System for Improved Outcome Prediction

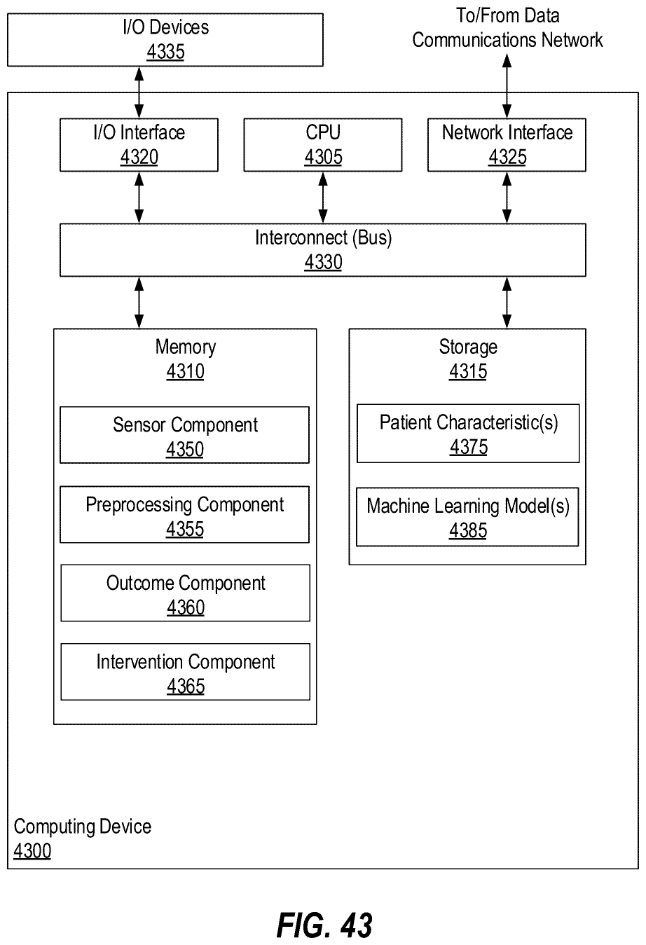

FIG. 43 depicts an example computing device 4300 configured to predict and/or learn to predict patient outcomes various aspects of the present disclosure. Although depicted as a physical device, in embodiments, the computing device 4300 may be implemented using virtual device(s), and/or across a number of devices (e.g., in a cloud environment). In one embodiment, the computing device 4300 corresponds to one or more systems that train and/or use machine learning models to predict patient outcomes, such as the monitoring system 105 of FIG. 1, the training system 520 of FIGS. 5-7, the inference component 810 and/or intervention component 820 of FIG. 8, and the like.

As illustrated, the computing device 4300 includes a CPU 4305, memory 4310, storage 4315, a network interface 4325, and one or more I/O interfaces 4320. In the illustrated embodiment, the CPU 4305 retrieves and executes programming instructions stored in memory 4310, as well as stores and retrieves application data residing in storage 4315. The CPU 4305 is generally representative of a single CPU and/or GPU, multiple CPUs and/or GPUs, a single CPU and/or GPU having multiple processing cores, and the like. The memory 4310 is generally included to be representative of a random access memory. Storage 4315 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 4335 (such as keyboards, monitors, etc.) are connected via the I/O interface(s) 4320. Further, via the network interface 4325, the computing device 4300 can be communicatively coupled with one or more other devices and components (e.g., via a network, which may include the Internet, local network(s), and the like). As illustrated, the CPU 4305, memory 4310, storage 4315, network interface(s) 4325, and I/O interface(s) 4320 are communicatively coupled by one or more buses 4330.

In the illustrated embodiment, the memory 4310 includes a sensor component 4350, a preprocessing component 4355, an outcome component 4360, and an intervention component 4365, which may perform one or more embodiments discussed above. Although depicted as discrete components for conceptual clarity, in embodiments, the operations of the depicted components (and others not illustrated) may be combined or distributed across any number of components. Further, although depicted as software residing in memory 4310, in embodiments, the operations of the depicted components (and others not illustrated) may be implemented using hardware, software, or a combination of hardware and software.

In some embodiments, the sensor component 4350 may be used to manage sensors, such as to activate and deactivate them, as discussed above. For example, the sensor component 4350 may be used to dynamically activate sensors when actions are being performed, and facilitate collection and/or storage of the sensor data. After action completion, the sensor component 4350 may selectively deactivate some or all of the sensors to preserve patient privacy and reduce computational expense, as discussed above.

In some embodiments, the preprocessing component 4355 may be used to process or preprocess sensor data, as discussed above. For example, the preprocessing component 4355 may be used to remove noise, smooth the data, delineate the data into windows of time, determine which data to collect or analyze, and the like.

In some embodiments, the outcome component 4360 may be used to predict potential patient outcomes that may result from action performance, as discussed above. For example, the outcome component 4360 may use machine learning models (e.g., the outcome machine learning model 525 of FIGS. 5-8, the refined outcome machine learning model 725 of FIG. 7, the position machine learning model 635 and/or the result machine learning model 640 of FIG. 6, and/or the machine learning model(s) 4385) to process sensor data in order to generate score(s) or other output indicating how probable one or more outcomes are for the patient, based on how the action was performed.

In some embodiments, the intervention component 4365 may be used to generate or facilitate interventions, as discussed above. For example, the intervention component 4365 may be used to evaluate the predicted outcomes using one or more criteria, and to initiate interventions such as notifying the user when the criteria are not satisfied.

In the illustrated example, the storage 4315 includes patient characteristics(s) 4375 and machine learning model(s) 4385. In one embodiment, the patient characteristic(s) 4375 may include various information such as ages, demographics, comorbidities, sensitivities, allergies, weight, height, gender, and the like. In some embodiments, some or all of the patient characteristics 4375 may be used as input to the machine learning models to improve model output. The machine learning model(s) 4385 generally correspond to one or more machine learning models that generate predicted outcome scores or measures based on input sensor data (e.g., the outcome machine learning model 525 of FIGS. 5-8, the refined outcome machine learning model 725 of FIG. 7, the position machine learning model 635 and/or the result machine learning model 640 of FIG. 6). Although depicted as residing in storage 4315, the patient characteristics 4375 and the machine learning models 4385 may be stored in any suitable location, including memory 4310.

### Example Computing System for Improved Injury Prediction

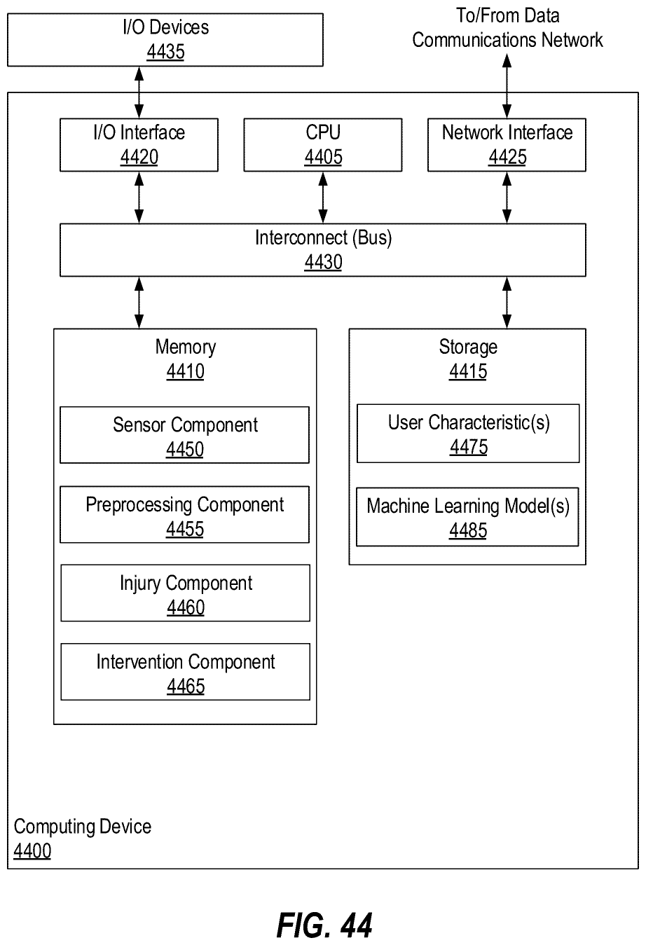

FIG. 44 depicts an example computing device 4400 configured to predict and/or learn to predict action injury risk various aspects of the present disclosure. Although depicted as a physical device, in embodiments, the computing device 4400 may be implemented using virtual device(s), and/or across a number of devices (e.g., in a cloud environment). In one embodiment, the computing device 4400 corresponds to one or more systems that train and/or use machine learning models to predict injury risks, such as the monitoring system 105 of FIG. 1, the training system 920 of FIGS. 9-10, the inference component 1110 and/or intervention component 1120 of FIG. 11, and the like.

As illustrated, the computing device 4400 includes a CPU 4405, memory 4410, storage 4415, a network interface 4425, and one or more I/O interfaces 4420. In the illustrated embodiment, the CPU 4405 retrieves and executes programming instructions stored in memory 4410, as well as stores and retrieves application data residing in storage 4415. The CPU 4405 is generally representative of a single CPU and/or GPU, multiple CPUs and/or GPUs, a single CPU and/or GPU having multiple processing cores, and the like. The memory 4410 is generally included to be representative of a random access memory. Storage 4415 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 4435 (such as keyboards, monitors, etc.) are connected via the I/O interface(s) 4420. Further, via the network interface 4425, the computing device 4400 can be communicatively coupled with one or more other devices and components (e.g., via a network, which may include the Internet, local network(s), and the like). As illustrated, the CPU 4405, memory 4410, storage 4415, network interface(s) 4425, and I/O interface(s) 4420 are communicatively coupled by one or more buses 4430.

In the illustrated embodiment, the memory 4410 includes a sensor component 4450, a preprocessing component 4455, an injury component 4460, and an intervention component 4465, which may perform one or more embodiments discussed above. Although depicted as discrete components for conceptual clarity, in embodiments, the operations of the depicted components (and others not illustrated) may be combined or distributed across any number of components. Further, although depicted as software residing in memory 4410, in embodiments, the operations of the depicted components (and others not illustrated) may be implemented using hardware, software, or a combination of hardware and software.

In some embodiments, the sensor component 4450 may be used to manage sensors, such as to activate and deactivate them, as discussed above. For example, the sensor component 4450 may be used to dynamically activate sensors when actions are being performed, and facilitate collection and/or storage of the sensor data. After action completion, the sensor component 4450 may selectively deactivate some or all of the sensors to preserve patient privacy and reduce computational expense, as discussed above.

In some embodiments, the preprocessing component 4455 may be used to process or preprocess sensor data, as discussed above. For example, the preprocessing component 4455 may be used to remove noise, smooth the data, delineate the data into windows of time, determine which data to collect or analyze, and the like.

In some embodiments, the injury component 4460 may be used to predict potential injuries that may result from action performance, as discussed above. For example, the injury component 4460 may use machine learning models (e.g., the injury machine learning model 925 of FIGS. 9-11, the refined injury machine learning model 1025 of FIG. 10, and/or the machine learning model(s) 4485) to process sensor data in order to generate score(s) or other output indicating how probable one or more injuries are for the user, based on how the action was performed.

In some embodiments, the intervention component 4465 may be used to generate or facilitate interventions, as discussed above. For example, the intervention component 4465 may be used to evaluate the predicted injury risks using one or more criteria, and to initiate interventions such as notifying the user when the criteria are not satisfied.

In the illustrated example, the storage 4415 includes user characteristics(s) 4475 and machine learning model(s) 4485. In one embodiment, the user characteristic(s) 4475 may include various information such as ages, demographics, comorbidities, weight, height, gender, previous injury records, and the like. In some embodiments, some or all of the user characteristics 4475 may be used as input to the machine learning models to improve model output. The machine learning model(s) 4485 generally correspond to one or more machine learning models that generate predicted injury scores or measures based on input sensor data (e.g., the injury machine learning model 925 of FIGS. 9-11, the refined injury machine learning model 1025 of FIG. 10). Although depicted as residing in storage 4415, the user characteristics 4475 and the machine learning models 4485 may be stored in any suitable location, including memory 4410.

Although the illustrated example depicts three distinct computing devices 4200, 4300, and 4400, in some aspects, some or all of these devices may be combined in a single system. That is, a single system may include the quality component 4260, the outcome component 4360, and/or the injury component 4460, and perform the operations discussed above.

### ADDITIONAL CONSIDERATIONS

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., the monitoring system 105 of FIG. 1) or related data available in the cloud. For example, the monitoring system could execute on a computing system in the cloud and automatically train and/or use machine learning models to predict action quality, patient outcomes, and/or injury risk, as discussed above. In such a case, the monitoring system could use machine learning to process sensor data to generate the predictions. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

### Example Clauses

Implementation examples are described in the following numbered clauses:

Clause 1: A method, comprising: accessing first sensor data collected by a set of sensors, the first sensor data indicating movement of a first user in a physical environment determining an action that the first user was performing when the first sensor data was collected, wherein the first user was performing the action to assist a patient; generating a quality score for performance of the action, by the first user, based on processing the first sensor data using a trained machine learning model; and in response to determining that the quality score does not satisfy one or more criteria, initiating one or more interventions for the first user.

Clause 2: The method of Clause 1, wherein the first sensor data comprises at least one of: accelerometer data, orientation data, pressure data, video data, image data, or audio data.

Clause 3: The method of any of Clauses 1-2, wherein determining the action that the first user was performing comprises receiving, from the first user, an indication that the first user is performing the action.

Clause 4: The method of Clause 3, wherein the first sensor data is accessed in response to receiving the indication that the first user is performing the action.

Clause 5: The method of any of Clauses 3-4, wherein: prior to receiving the indication, at least one sensor of the set of sensors is in a deactivated state, and subsequent to determining that the first user has completed the action, the at least one sensor is returned to the deactivated state.

Clause 6: The method of any of Clauses 1-5, wherein the trained machine learning model was trained based on a set of action exemplars, each respective action exemplar of the set of action exemplars comprising respective sensor data collected while a respective user performed the action correctly.

Clause 7: The method of any of Clauses 1-6, further comprising selecting the trained machine learning model, from a set of trained machine learning models, based on the action.

Clause 8: The method of any of Clauses 1-7, wherein the quality score is generated further based on processing an indication of the action using the trained machine learning model.

Clause 9: The method of any of Clauses 1-8, wherein initiating the one or more interventions comprises at least one of: (i) transmitting a notification, indicating the action, to a supervising user, or (ii) transmitting a tutorial, to the first user, indicating how to correctly perform the action.

Clause 10: A method, comprising: accessing first sensor data collected by a set of sensors, the first sensor data indicating movement of a first user in a physical environment; determining an action that the first user was performing when the first sensor data was collected, wherein the first user was performing the action to assist a patient; training a machine learning model to generate quality scores for performance of the action based on the first sensor data; and deploying the machine learning model to generate quality scores.

Clause 11: The method of Clause 10, wherein the first sensor data is accessed in response to determining that the first user performed the action in accordance with a preferred technique for performing the action.

Clause 12: A method, comprising: accessing first sensor data collected by a set of sensors, the first sensor data indicating positioning of a patient in a physical environment; determining a set of patient characteristics for the patient; generating an outcome score for the positioning of the patient, using a first trained machine learning model, based on the first sensor data and the set of patient characteristics; and in response to determining that the outcome score does not satisfy one or more criteria, initiating one or more interventions for the patient.

Clause 13: The method of Clause 12, wherein the first sensor data comprises at least one of: accelerometer data, orientation data, pressure data, video data, image data, or audio data.

Clause 14: The method of any of Clauses 12-13, wherein the first sensor data is accessed in response to receiving an indication, from a user, that the user is performing an action to reposition the patient.

Clause 15: The method of Clause 14, wherein: prior to receiving the indication, at least one sensor of the set of sensors is in a deactivated state, and subsequent to determining that the user has completed the action, the at least one sensor is returned to the deactivated state.

Clause 16: The method of any of Clauses 12-15, wherein the set of patient characteristics comprise at least one of demographics of the patient, or one or more medical conditions of the patient.

Clause 17: The method of any of Clauses 12-16, wherein the first trained machine learning model was trained based on a set of position exemplars, each respective position exemplar of the set of position exemplars comprising respective sensor data and respective outcome data for a corresponding patient.

Clause 18: The method of any of Clauses 12-17, wherein generating the outcome score comprises: predicting the positioning of the patient based on processing the sensor data using a second trained machine learning model; and processing the positioning of the patient using the first trained machine learning model.

Clause 19: The method of any of Clauses 12-18, wherein the outcome score indicates at least one of: (i) a probability that the positioning of the patient will be comfortable for the patient, or (ii) a prediction of whether the positioning of the patient will improve or worsen one or more medical conditions of the patient.

Clause 20: The method of any of Clauses 12-19, wherein initiating the one or more interventions comprises transmitting a notification to a user assisting the patient.

Clause 21: A method, comprising: accessing first sensor data collected by a set of sensors, the first sensor data indicating positioning of a patient in a physical environment;

determining a set of patient characteristics for the patient; training a first machine learning model to generate outcome scores for patient positioning based on the first sensor data and the set of patient characteristics; and deploying the first machine learning model to generate outcome scores.

Clause 22: The method of Clause 21, further comprising training a machine learning model to generate predictions for patient positioning based on the first sensor data, wherein the first machine learning model uses patient positioning as input.

Clause 23: A method, comprising: accessing first sensor data collected by a set of sensors, the first sensor data indicating movement of a first user in a physical environment; determining an action that the first user was performing when the first sensor data was collected, wherein the first user was performing the action to assist a patient; generating an injury risk score for performance of the action, by the first user, based on processing the first sensor data using a trained machine learning model, wherein the injury risk score indicates a risk of injury to the first user; and in response to determining that the injury risk score satisfies one or more criteria, initiating one or more interventions for the first user.

Clause 24: The method of Clause 23, wherein the first sensor data comprises at least one of: accelerometer data, orientation data, pressure data, video data, image data, or audio data.

Clause 25: The method of any of Clauses 23-24, wherein: determining the action that the first user was performing comprises receiving, from the first user, an indication that the first user is performing the action, and the first sensor data is accessed in response to receiving the indication that the first user is performing the action.

Clause 26: The method of any of Clauses 23-25, wherein the trained machine learning model was trained based on a set of action exemplars, each respective action exemplar of the set of action exemplars comprising respective sensor data collected while a respective user performed the action correctly.

Clause 27: The method of any of Clauses 23-26, further comprising selecting the trained machine learning model, from a set of trained machine learning models, based on the action.

Clause 28: The method of any of Clauses 23-27, wherein generating the injury risk score is performed in response to determining that the first user is in an injury recovery state based on a prior injury.

Clause 29: The method of Clause 28, wherein determining that the first user is in an injury recovery state comprises determining that the prior injury occurred within a defined period of time.

Clause 30: The method of any of Clauses 28-29, wherein determining that the first user is in the injury recovery state comprises determining that the first user is working a return to work shift after the prior injury.

Clause 31: The method of any of Clauses 23-30, wherein initiating the one or more interventions comprises at least one of: (i) transmitting a notification, indicating the injury risk score, to a supervising user, or (ii) transmitting a notification, to the first user, indicating the injury risk score.

Clause 32: A method, comprising: accessing first sensor data collected by a set of sensors, the first sensor data indicating movement of a first user in a physical environment; determining an action that the first user was performing when the first sensor data was collected, wherein the first user was performing the action to assist a patient; training a machine learning model to generate injury risk scores for performance of the action based on the first sensor data; and deploying the machine learning model to generate injury risk scores.

Clause 33: The method of Clause 32, wherein the first sensor data is accessed in response to determining that the first user performed the action while in an injury recovery state.

Clause 34: A system, comprising: one or more processors; and one or more memories storing a program, which, when executed on any combination of the one or more processors, performs operations, the operations in accordance with any of Clauses 1-33.

Clause 35: A system, comprising means for performing a method in accordance with any of Clauses 1-34.

Clause 36: A non-transitory computer-readable medium comprising computer-executable instructions that, when executed by one or more processors of a processing system, cause the processing system to perform a method in accordance with any of Clauses 1-34.

Clause 37: A computer program product embodied on a computer-readable storage medium comprising code for performing a method in accordance with any of Clauses 1-34.

What is claimed is:

1. A method, comprising:
accessing first sensor data collected by a set of sensors, the first sensor data indicating positioning of a patient in a physical environment;
determining a set of patient characteristics for the patient;
generating an outcome score for the positioning of the patient, using a first trained machine learning model, based on the first sensor data and the set of patient characteristics; and
in response to determining that the outcome score does not satisfy one or more criteria, initiating one or more interventions for the patient.

2. The method of claim 1, wherein the first sensor data comprises at least one of: accelerometer data, orientation data, pressure data, video data, image data, or audio data.

3. The method of claim 1, wherein the first sensor data is accessed in response to receiving an indication, from a user, that the user is performing an action to reposition the patient.

4. The method of claim 3, wherein:
prior to receiving the indication, at least one sensor of the set of sensors is in a deactivated state, and
subsequent to determining that the user has completed the action, the at least one sensor is returned to the deactivated state.

5. The method of claim 1, wherein the set of patient characteristics comprise at least one of demographics of the patient, or one or more medical conditions of the patient.

6. The method of claim 1, wherein the first trained machine learning model was trained based on a set of position exemplars, each respective position exemplar of the set of position exemplars comprising respective sensor data and respective outcome data for a corresponding patient.

7. The method of claim 1, wherein generating the outcome score comprises:
predicting the positioning of the patient based on processing the sensor data using a second trained machine learning model; and
processing the positioning of the patient using the first trained machine learning model.

8. The method of claim 1, wherein the outcome score indicates at least one of:
(i) a probability that the positioning of the patient will be comfortable for the patient, or
(ii) a prediction of whether the positioning of the patient will improve or worsen one or more medical conditions of the patient.

9. The method of claim 1, wherein initiating the one or more interventions comprises transmitting a notification to a user assisting the patient.

10. A method, comprising:
accessing first sensor data collected by a set of sensors, the first sensor data indicating positioning of a patient in a physical environment;
determining a set of patient characteristics for the patient;
training a first machine learning model to generate outcome scores for patient positioning based on the first sensor data and the set of patient characteristics; and
deploying the first machine learning model to generate outcome scores.

11. The method of claim 10, further comprising training a machine learning model to generate predictions for patient positioning based on the first sensor data, wherein the first machine learning model uses patient positioning as input.

12. A system, comprising:
one or more processors; and
one or more memories storing a program, which, when executed on any combination of the one or more processors, performs operations, the operations comprising:
accessing first sensor data collected by a set of sensors, the first sensor data indicating positioning of a patient in a physical environment;
determining a set of patient characteristics for the patient;
generating an outcome score for the positioning of the patient, using a first trained machine learning model, based on the first sensor data and the set of patient characteristics; and
in response to determining that the outcome score does not satisfy one or more criteria, initiating one or more interventions for the patient.

13. The system of claim 12, wherein the first sensor data comprises at least one of: accelerometer data, orientation data, pressure data, video data, image data, or audio data.

14. The system of claim 12, wherein the first sensor data is accessed in response to receiving an indication, from a user, that the user is performing an action to reposition the patient.

15. The system of claim 14, wherein:
prior to receiving the indication, at least one sensor of the set of sensors is in a deactivated state, and
subsequent to determining that the user has completed the action, the at least one sensor is returned to the deactivated state.

16. The system of claim 12, wherein the set of patient characteristics comprise at least one of demographics of the patient, or one or more medical conditions of the patient.

17. The system of claim 12, wherein the first trained machine learning model was trained based on a set of position exemplars, each respective position exemplar of the set of position exemplars comprising respective sensor data and respective outcome data for a corresponding patient.

18. The system of claim 12, wherein generating the outcome score comprises:
predicting the positioning of the patient based on processing the sensor data using a second trained machine learning model; and
processing the positioning of the patient using the first trained machine learning model.

19. The system of claim 12, wherein the outcome score indicates at least one of:
(i) a probability that the positioning of the patient will be comfortable for the patient, or
(ii) a prediction of whether the positioning of the patient will improve or worsen one or more medical conditions of the patient.

20. The system of claim 12, wherein initiating the one or more interventions comprises transmitting a notification to a user assisting the patient.
